(19) United States
(12) Patent Application Publication
(10) Pub. No.: US 2026/0079394 A1
(43) Pub. Date: Mar. 19, 2026

(54) RESIST COMPOSITION, METHOD FOR FORMING RESIST PATTERN, COMPOUND AND ACID DIFFUSION CONTROL AGENT

(71) Applicant: TOKYO OHKA KOGYO CO., LTD., Kawasaki-shi (JP)

(72) Inventors: Tatsuya FUJII, Kawasaki-shi (JP); KhanhTin NGUYEN, Kawasaki-shi (JP)

(21) Appl. No.: 19/108,688

(22) PCT Filed: Sep. 26, 2023
(86) PCT No.: PCT/JP2023/034959

§ 371 Date: Mar. 4, 2025

(30) Foreign Application Priority Data
Sep. 27, 2022 (JP) 2022-153680

(51) Int. Cl. G03F 7/004; C07C 59/88; C07C 59/90; C07C 381/12; C07D 333/76; G03F 7/00; G03F 7/038; G03F 7/039

(52) U.S. Cl. CPC G03F 7/0045; C07C 59/88; C07C 59/90; C07C 381/12; C07D 333/76; G03F 7/0382; G03F 7/0384; G03F 7/0395; G03F 7/70033

(57) ABSTRACT

A resist composition containing a resin component whose solubility in a development solution changes under the action of the acid and a compound represented by General Formula (d0). In the formula, Ar is an aromatic ring; Xd is an iodine atom, a fluorine atom, a bromine atom or a fluorinated alkyl group; Rd is a substituent; nd is an integer of 1 or more as long as the valence allows, and md is an integer of 0 or more as long as the valence allows; Ld is a single bond or a divalent linking group; m is an integer of 1 or more, and M^{m+} is an m-valent cation

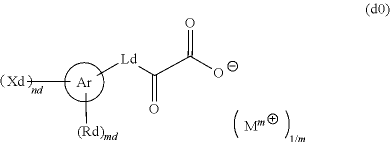

### TECHNICAL FIELD

The present invention relates to a resist composition, a method for forming a resist pattern, a compound and an acid diffusion control agent.

Priority is claimed on Japanese Patent Application No. 2022-153680, filed Sep. 27, 2022, the content of which is incorporated herein by reference.

### BACKGROUND ART

In recent years, in the production of semiconductor elements and liquid crystal display elements, advances in lithography technology have led to rapid progress in refinement of patterns. In refinement methods, exposure light sources with shorter wavelengths (higher energy) are generally used.

Resist materials are required to have lithography properties such as sensitivity to these exposure light sources and a resolution at which fine pattern dimensions can be reproduced.

As a resist material that satisfies such requirements, a chemically amplified resist composition that contains a base component whose solubility in a development solution changes under the action of an acid and an acid generator component that generates an acid upon exposure has been conventionally used.

For example, Patent Document 1 discloses a resist composition including an acid generator having a fused cyclic group containing a fused ring containing one or more aromatic rings in the anion moiety and an acid diffusion control agent having a polar linking group in the anion moiety in combination. It discloses that this resist composition allows a resist pattern with reduced roughness and a favorable fine resolution to be formed.

### CITATION LIST

### Patent Document

Patent Document 1: Japanese Unexamined Patent Application, First Publication No. 2021-103230

### SUMMARY OF INVENTION

### Technical Problem

With further advancement of lithography technology, and expansion of application fields, refinement of patterns is rapidly progressing. Accordingly, when semiconductor elements and the like are produced, a technology capable of forming fine patterns with favorable shapes is required. For example, in lithography using EUV or EB, the object is to form fine patterns of several tens of nm.

In the resist composition containing a carboxylate acid diffusion control agent as described in Patent Document 1, there is room for improvement in lithography properties such as sensitivity and resolution.

The present invention has been made in view of the above circumstances and an object of the present invention is to provide a resist composition that allows high sensitivity and favorable lithography properties to be obtained, a method for forming a resist pattern using the resist composition, a compound that can be used in the production of the resist composition, and an acid diffusion control agent containing the compound.

### Solution to Problem

In order to achieve the above object, the present invention provides the following aspects.

Specifically, a first aspect of the present invention is a resist composition which generates an acid upon exposure and whose solubility in a development solution changes under the action of the acid, including: a resin component (A1) whose solubility in a development solution changes under the action of the acid; and a compound (D0) represented by the following General Formula (d0):

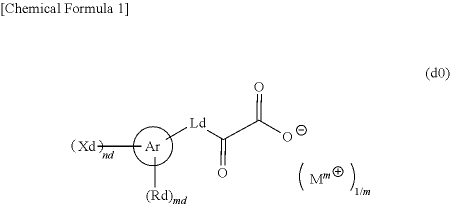

[in the formula, Ar is an aromatic ring; Xd is an iodine atom, a fluorine atom, a bromine atom or a fluorinated alkyl group; Rd is a substituent; nd is an integer of 1 or more as long as the valence allows, and md is an integer of 0 or more as long as the valence allows; Ld is a single bond or a divalent linking group; when nd is an integer of 2 or more, a plurality of Xd's may be the same as or different from each other; when md is an integer of 2 or more, a plurality of Rd's may be the same as or different from each other; and m is an integer of 1 or more, and M^{m+} is an m-valent cation].

A second aspect of the present invention is a method for forming a resist pattern, including a step of forming a resist film on a support using the resist composition according to the first aspect; a step of exposing the resist film; and a step of developing the exposed resist film to form a resist pattern.

A third aspect of the present invention is a compound represented by the following General Formula (d0).

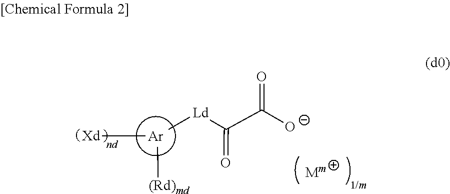

[in the formula, Ar is an aromatic ring; Xd is an iodine atom, a fluorine atom, a bromine atom or a fluorinated alkyl group; Rd is a substituent; nd is an integer of 1 or more as long as the valence allows, and md is an integer of 0 or more as long as the valence allows; Ld is a single bond or a divalent linking group; when nd is an integer of 2 or more, a plurality of Xd's may be the same as or different from each other; when md is an integer of 2 or more, a plurality of Rd's may be the same as or different from each other; and m is an integer of 1 or more, and M^{m+} is an m-valent cation].

A fourth aspect of the present invention is an acid diffusion control agent containing the compound according to the third aspect.

### Advantageous Effects of Invention

According to the present invention, it is possible to provide a resist composition that allows high sensitivity and favorable lithography properties to be obtained, a method for forming a resist pattern using the resist composition, a compound that can be used in the production of the resist composition, and an acid diffusion control agent containing the compound.

### DESCRIPTION OF EMBODIMENTS

In the scope of this specification and claims, the term “aliphatic” is a relative concept with respect to the term “aromatic,” and defines a group, compound or the like which has no aromaticity.

Unless otherwise specified, the term “alkyl group” includes linear, branched and cyclic monovalent saturated hydrocarbon groups. The same applies to alkyl groups in alkoxy groups.

Unless otherwise specified, the term “alkylene group” includes linear, branched and cyclic divalent saturated hydrocarbon groups.

The term “halogen atom” includes a fluorine atom, a chlorine atom, a bromine atom, an iodine atom, and the like.

The term “structural unit” refers to a monomer unit (monomeric unit) constituting a high-molecular-weight compound (a resin, a polymer, or a copolymer).

The description “may have a substituent” includes both a case in which a hydrogen atom (—H) is substituted with a monovalent group and a case in which a methylene group (—CH₂—) is substituted with a divalent group.

The term “exposure” is a concept that includes irradiation with any form of radiation.

The term “acid-decomposable group” refers to a group having acid-decomposability in which at least some bonds in the structure of the acid-decomposable group can be cleaved under the action of an acid.

Examples of acid-decomposable groups whose polarity increases under the action of an acid include groups that decompose under the action of an acid to generate a polar group.

Examples of polar groups include a carboxy group, a hydroxyl group, an amino group, and a sulfo group (—SO₃H).

More specific examples of acid-decomposable groups include groups in which the polar group is protected with an acid-dissociable group (for example, a group in which a hydrogen atom of an OH-containing polar group is protected with an acid-dissociable group).

The term “acid-dissociable group” refers to both (i) a group having acid-dissociability in which the bond between the acid-dissociable group and an atom adjacent to the acid-dissociable group can be cleaved under the action of an acid and (ii) a group in which some bonds are cleaved under the action of an acid, then a decarboxylation reaction additionally occurs, and thus the bond between the acid-dissociable group and an atom adjacent to the acid-dissociable group can be cleaved.

The acid-dissociable group constituting the acid-decomposable group needs to be a group having a lower polarity than the polar group generated by dissociation of the acid-dissociable group, and accordingly, when the acid-dissociable group is dissociated under the action of an acid, a polar group having a higher polarity than the acid-dissociable group is generated and the polarity increases. As a result, the polarity of the entire component (A1) increases. When the polarity increases, the relative solubility in the development solution changes, when the development solution is an alkaline development solution, the solubility increases, and when the development solution is an organic development solution, the solubility decreases.

The term “base component” is an organic compound having a film-forming ability. Organic compounds used as the base component are broadly classified into non-polymers and polymers. Generally, regarding the non-polymer, one having a molecular weight of 500 or more and less than 4,000 is used (hereinafter referred to as a “low-molecular-weight compound”). Hereinafter, the “resin,” “high-molecular-weight compound” or “polymer” refers to a polymer having a molecular weight of 1,000 or more. Regarding the molecular weight of the polymer, the weight average molecular weight in terms of polystyrene determined through gel permeation chromatography (GPC) is used.

The term “derived structural unit” refers to a structural unit formed by cleavage of multiple bonds between carbon atoms, for example, an ethylenic double bond.

In the “acrylic acid ester,” a hydrogen atom bonded to the carbon atom at the α-position may be substituted with a substituent. The substituent (R^{αx}) that substitutes a hydrogen atom bonded to the carbon atom at the α-position is an atom or group other than a hydrogen atom. In addition, it also includes an itaconic acid diester in which the substituent (R^{αx}) is substituted with a substituent containing an ester bond and an α-hydroxyacrylic ester in which the substituent (R^{αx}) is substituted with a hydroxyalkyl group or a group modified with a hydroxyl group. Here, unless otherwise specified, the carbon atom at the α-position of the acrylic acid ester is a carbon atom to which a carbonyl group of acrylic acid is bonded.

Hereinafter, an acrylic acid ester in which a hydrogen atom bonded to the carbon atom at the α-position is substituted with a substituent will sometimes be referred to as an α-substituted acrylic acid ester.

The term “derivative” is a concept including a compound in which the hydrogen atom at the α-position of a target compound is substituted with another substituent such as an alkyl group or a halogenated alkyl group and derivatives thereof. Examples of such derivatives include those in which a hydrogen atom of a hydroxyl group of a target compound in which the hydrogen atom at the α-position may be substituted with a substituent is substituted with an organic group; and those in which a substituent other than a hydroxyl group is bonded to a target compound in which the hydrogen atom at the α-position may be substituted with a substituent. Here, unless otherwise specified, the carbon atom at the α-position refers to a first carbon atom adjacent to a functional group.

Examples of substituents that substitute the hydrogen atom at the α-position of hydroxystyrene include the same substituents as for R^{αx}.

In the scope of this specification and claims, some structures represented by chemical formulae may have asymmetric carbons, and may have enantiomers or diastereomers. In such cases, one chemical formula represents the isomers. These isomers may be used alone or as a mixture.

### (Resist Composition)

The resist composition of the present embodiment generates an acid upon exposure and its solubility in a development solution changes under the action of the acid.

Such a resist composition contains a base component (A) whose solubility in a development solution changes under the action of an acid (hereinafter referred to as a “component (A)”), and a compound (D0) represented by General Formula (d0) (hereinafter referred to as a “component (D0)”) described below.

In addition, the resist composition of the present embodiment may further contain other components in addition to the above component (A) and component (D0). Examples of other components include the following component (B), component (D), component (E), component (F), and component (S).

In the resist composition of the present embodiment, the component (A) may generate an acid upon exposure, and an additive component added separately from the component (A) may generate an acid upon exposure.

Specifically, the resist composition of the present embodiment may be (1) one that further contains an acid generator component (B) that generates an acid upon exposure (hereinafter referred to as a “component (B)”); (2) one in which the component (A) is a component that generates an acid upon exposure; or (3) one in which the component (A) is a component that generates an acid upon exposure, and which further contains a component (B).

That is, in the above cases (2) and (3), the component (A) is “a base component that generates an acid upon exposure and its solubility in a development solution changes under the action of the acid.” When the component (A) is a base component that generates an acid upon exposure and its solubility in a development solution changes under the action of the acid, the component (A1) described below is preferably a resin that generates an acid upon exposure and its solubility in a development solution changes under the action of the acid. As such a resin, a high-molecular-weight compound having a structural unit that generates an acid upon exposure can be used. As the structural unit that generates an acid upon exposure, known structural units can be used.

Among the above examples, the resist composition of the present embodiment is preferably the above case (1). That is, the resist composition of the present embodiment preferably contains the component (A) and the component (B).

When a resist film is formed using the resist composition of the present embodiment and the resist film is subjected to selective exposure, in an exposed part of the resist film, for example, an acid is generated from the component (B), and the solubility of the component (A) in a development solution changes under the action of the acid, but in an unexposed part of the resist film, since the solubility of the component (A) in a development solution does not change, a difference in solubility in a development solution occurs between the exposed part and the unexposed part. Therefore, when the resist film is developed, if the resist composition is of a positive type, the exposed part of the resist film is dissolved and removed to form a positive type resist pattern, and if the resist composition is of a negative type, the unexposed part of the resist film is dissolved and removed to form a negative type resist pattern.

The resist composition of the present embodiment may be a positive type resist composition or a negative type resist composition. In addition, the resist composition of the present embodiment may be used for an alkaline development process in which an alkaline development solution is used in a development treatment during resist pattern formation or may be used for a solvent development process in which a development solution containing an organic solvent (organic development solution) is used in the development treatment.

### <Component (A)>

In the resist composition of the present embodiment, the component (A) preferably contains a resin component (A1) whose solubility in a development solution changes under the action of the acid (hereinafter referred to as a “component (A1)”).

When the component (A1) is used, since the polarity of the base component changes before and after exposure, a favorable development contrast can be obtained not only in the alkaline development process but also in the solvent development process.

As the component (A), other high-molecular-weight compounds and/or low-molecular-weight compounds may be used in combination with the component (A1).

The component (A) may be a “base component that generates an acid upon exposure and its solubility in a development solution changes under the action of the acid.” When the component (A) is a base component that generates an acid upon exposure and its solubility in a development solution changes under the action of the acid, the component (A1) is preferably a resin that generates an acid upon exposure and its solubility in a development solution changes under the action of the acid. As such a resin, a high-molecular-weight compound having a structural unit that generates an acid upon exposure can be used. As the structural unit that generates an acid upon exposure, known structural units can be used.

In the resist composition of the present embodiment, the components (A) may be used alone or two or more thereof may be used in combination.

### Component (A1)

The component (A1) is a resin component whose solubility in a development solution changes under the action of an acid.

The component (A1) preferably has a structural unit (a1) having an acid-decomposable group whose polarity increases under the action of an acid.

The component (A1) may have, as necessary, other structural units, in addition to the structural unit (a1).

<<Structural Unit (a1)>>

The structural unit (a1) is a structural unit having an acid-decomposable group whose polarity increases under the action of an acid.

Examples of acid-dissociable groups include those that have been previously proposed as acid-dissociable groups in base resins for chemically amplified resist compositions.

Specific examples of those proposed as acid-dissociable groups in base resins for chemically amplified resist compositions include the following “acetal-type acid-dissociable group,” “tertiary alkyl ester type acid-dissociable group,” and “tertiary alkyloxycarbonyl acid-dissociable group.”

### Acetal-Type Acid-Dissociable Group:

Examples of acid-dissociable groups that protect a carboxy group or a hydroxyl group among the polar groups include an acid-dissociable group represented by the following General Formula (a1-r-1) (hereinafter sometimes referred to as an “acetal-type acid-dissociable group”).

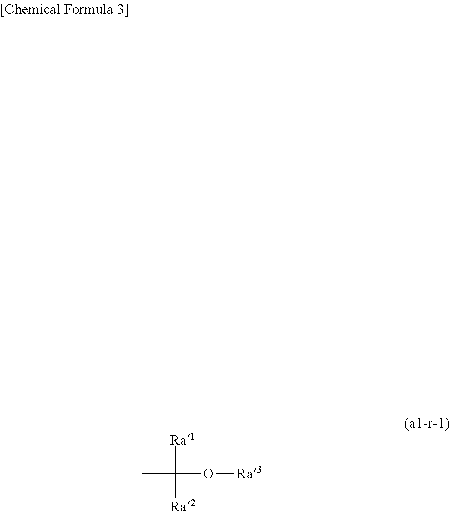

[in the formula, Ra′¹ and Ra′² are a hydrogen atom or an alkyl group, Ra′³ is a hydrocarbon group, and Ra′³ may be bonded to either Ra′¹ or Ra′² to form a ring].

In Formula (a1-r-1), it is preferable that at least one of Ra′¹ and Ra′² be a hydrogen atom, and it is more preferable that both of Ra′¹ and Ra′² be a hydrogen atom. When Ra′¹ or Ra′² is an alkyl group, as the alkyl group, the same alkyl groups exemplified as the substituent that may be bonded to the carbon atom at the α-position in the description of the α-substituted acrylic acid ester may be exemplified, and an alkyl group having 1 to 5 carbon atoms is preferable. Specifically, linear or branched alkyl groups are preferably exemplified. More specific examples thereof include a methyl group, an ethyl group, a propyl group, an isopropyl group, an n-butyl group, an isobutyl group, a tert-butyl group, a pentyl group, an isopentyl group, and a neopentyl group. A methyl group or an ethyl group is more preferable, and a methyl group is particularly preferable.

In Formula (a1-r-1), examples of hydrocarbon groups for Ra′³ include a linear or branched alkyl group and a cyclic hydrocarbon group.

The linear alkyl group preferably has 1 to 5 carbon atoms, more preferably has 1 to 4 carbon atoms, and still more preferably has 1 or 2 carbon atoms. Specific examples thereof include a methyl group, an ethyl group, an n-propyl group, an n-butyl group, and an n-pentyl group. Among these, a methyl group, an ethyl group or an n-butyl group is preferable, and a methyl group or an ethyl group is more preferable.

The branched alkyl group preferably has 3 to 10 carbon atoms and more preferably has 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group, and an isopropyl group is preferable.

When Ra′³ is a cyclic hydrocarbon group, the hydrocarbon group may be an alicyclic hydrocarbon group or an aromatic hydrocarbon group, and may be a polycyclic group or a monocyclic group.

The alicyclic hydrocarbon group which is a monocyclic group is preferably a group in which one hydrogen atom has been removed from a monocycloalkane. The monocycloalkane preferably has 3 to 6 carbon atoms, and specific examples thereof include cyclopentane and cyclohexane.

The alicyclic hydrocarbon group which is a polycyclic group is preferably a group in which one hydrogen atom has been removed from a polycycloalkane, and the polycycloalkane preferably has 7 to 12 carbon atoms, and specific examples thereof include adamantane, norbornane, isobornane, tricyclodecane, and tetracyclododecane.

When the cyclic hydrocarbon group for Ra′³ is an aromatic hydrocarbon group, the aromatic hydrocarbon group is a hydrocarbon group having at least one aromatic ring.

The aromatic ring is not particularly limited as long as it is a cyclic conjugated system having 4n+2 π electrons, and may be monocyclic or polycyclic. The aromatic ring preferably has 5 to 30 carbon atoms, more preferably has 5 to 20 carbon atoms, still more preferably has 6 to 15 carbon atoms, and particularly preferably 6 to 12 carbon atoms.

Specific examples of aromatic rings include aromatic hydrocarbon rings such as benzene, naphthalene, anthracene, and phenanthrene; and aromatic heterocycles in which some carbon atoms constituting the aromatic hydrocarbon ring are substituted with heteroatoms. Examples of heteroatoms in aromatic heterocycles include an oxygen atom, a sulfur atom, and a nitrogen atom. Specific examples of aromatic heterocycles include a pyridine ring and a thiophene ring.

Specific examples of aromatic hydrocarbon groups for Ra′³ include a group in which one hydrogen atom has been removed from the aromatic hydrocarbon ring or aromatic heterocycle (an aryl group or a heteroaryl group); a group in which one hydrogen atom has been removed from an aromatic compound containing two or more aromatic rings (for example, biphenyl, fluorine, etc.); and a group in which one hydrogen atom of the aromatic hydrocarbon ring or aromatic heterocycle is substituted with an alkylene group (for example, arylalkyl groups such as a benzyl group, a phenethyl group, a 1-naphthyl methyl group, a 2-naphthyl methyl group, a 1-naphthyl ethyl group, and a 2-naphthyl ethyl group). The alkylene group bonded to the aromatic hydrocarbon ring or aromatic heterocycle preferably has 1 to 4 carbon atoms, more preferably has 1 to 2 carbon atoms, and particularly preferably has 1 carbon atom.

The cyclic hydrocarbon group for Ra′³ may have a substituent. Examples of substituents include —R^{P1}, —R^{P2}—O—R^{P1}, —R^{P2}—CO—R^{P1}, —R^{P2}—CO—OR^{P1}, —R^{P2}—O—CO—R^{P1}, —R^{P2}—OH, —R^{P2}—CN and —R^{P2}—COOH (hereinafter these substituents are collectively referred to as “Ra^{x5}”).

Here, R^{P1} is a monovalent chain saturated hydrocarbon group having 1 to 10 carbon atoms, a monovalent aliphatic cyclic saturated hydrocarbon group having 3 to 20 carbon atoms or a monovalent aromatic hydrocarbon group having 6 to 30 carbon atoms. In addition, R^{P2} is a single bond, a divalent chain saturated hydrocarbon group having 1 to 10 carbon atoms, a divalent aliphatic cyclic saturated hydrocarbon group having 3 to 20 carbon atoms or a divalent aromatic hydrocarbon group having 6 to 30 carbon atoms. Here, some or all of the hydrogen atoms in the chain saturated hydrocarbon group, the aliphatic cyclic saturated hydrocarbon group and the aromatic hydrocarbon group for R^{P1} and R^{P2} may be substituted with fluorine atoms. The aliphatic cyclic hydrocarbon group may have one or more of one type of the substituents or may have one or more of each type of the plurality of substituents.

Examples of monovalent chain saturated hydrocarbon groups having 1 to 10 carbon atoms include a methyl group, an ethyl group, a propyl group, a butyl group, a pentyl group, a hexyl group, a heptyl group, an octyl group, and a decyl group.

Examples of monovalent aliphatic cyclic saturated hydrocarbon groups having 3 to 20 carbon atoms include monocyclic aliphatic saturated hydrocarbon groups such as a cyclopropyl group, a cyclobutyl group, a cyclopentyl group, a cyclohexyl group, a cycloheptyl group, a cyclooctyl group, a cyclodecyl group, and a cyclododecyl group; and polycyclic aliphatic saturated hydrocarbon groups such as a bicyclo[2.2.2]octanyl group, a tricyclo[5.2.1.02,6]decanyl group, a tricyclo[3.3.1.13,7]decanyl group, a tetracyclo[6.2.1.13,6.02,7]dodecanyl group, and an adamantyl group.

Examples of monovalent aromatic hydrocarbon groups having 6 to 30 carbon atoms include groups in which one hydrogen atom has been removed from an aromatic hydrocarbon ring such as benzene, biphenyl, fluorene, naphthalene, anthracene, and phenanthrene.

When Ra′³ is bonded to either Ra′¹ or Ra′² to form a ring, the cyclic group is preferably a 4- to 7-membered ring and more preferably a 4- to 6-membered ring. Specific examples of cyclic groups include a tetrahydropyranyl group and a tetrahydrofuranyl group.

### Tertiary Alkyl Ester Type Acid-Dissociable Group:

Examples of acid-dissociable groups that protect a carboxy group among the above polar groups include an acid-dissociable group represented by the following General Formula (a1-r-2).

Here, among the acid-dissociable groups represented by the following Formula (a1-r-2), one constituted by an alkyl group will sometimes be referred to hereinafter as a “tertiary alkyl ester type acid-dissociable group,” for convenience.

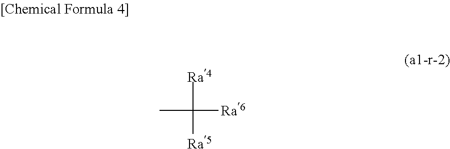

[in the formula, Ra′⁴ to Ra′⁶ are each a hydrocarbon group, and Ra′⁵ and Ra′⁶ may be bonded to each other to form a ring].

Examples of hydrocarbon groups for Ra′⁴ include a linear or branched alkyl group, a chain or cyclic alkenyl group, a chain alkynyl group, and a cyclic hydrocarbon group.

Examples of linear or branched alkyl groups and cyclic hydrocarbon groups (an alicyclic hydrocarbon group which is a monocyclic group, an alicyclic hydrocarbon group which is a polycyclic group, and an aromatic hydrocarbon group) for Ra′⁴ include the same groups as for Ra′³.

The chain or cyclic alkenyl group for Ra′⁴ is preferably an alkenyl group having 2 to 10 carbon atoms.

Examples of hydrocarbon groups for Ra′⁵ and Ra′⁶ include the same groups as for Ra′³.

When Ra′⁵ and Ra′⁶ are bonded to each other to form a ring, preferable examples thereof include a group represented by the following General Formula (a1-r2-1), a group represented by the following General Formula (a1-r2-2), and a group represented by the following General Formula (a1-r2-3).

On the other hand, when Ra′⁴ to Ra′⁶ are not bonded to each other and are independently a hydrocarbon group, preferable examples thereof include a group represented by the following General Formula (a1-r2-4).

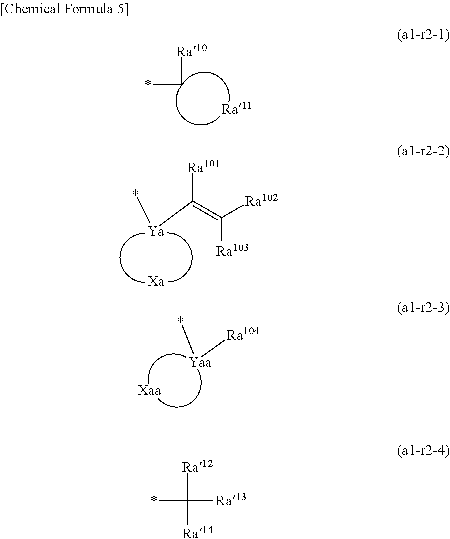

[in Formula (a1-r2-1), Ra′¹⁰ is a linear or branched alkyl group having 1 to 12 carbon atoms, some of which may be substituted with a halogen atom or a heteroatom-containing group, Ra′¹¹ is a group that forms an aliphatic cyclic group together with a carbon atom to which Ra′¹⁰ is bonded, in Formula (a1-r2-2), Ya is a carbon atom, Xa is a group which forms a cyclic hydrocarbon group together with Ya, some or all of the hydrogen atoms in the cyclic hydrocarbon group may be substituted, Ra¹⁰¹ to Ra¹⁰³ are each independently a hydrogen atom, a monovalent chain saturated hydrocarbon group having 1 to 10 carbon atoms or a monovalent aliphatic cyclic saturated hydrocarbon group having 3 to 20 carbon atoms, some or all of the hydrogen atoms in the chain saturated hydrocarbon group and the aliphatic cyclic saturated hydrocarbon group may be substituted, two or more of Ra¹⁰¹ to Ra¹⁰³ may be bonded to each other to form a ring structure, in Formula (a1-r2-3), Yaa is a carbon atom, Xaa is a group which forms an aliphatic cyclic group together with Yaa, Ra¹⁰⁴ is an aromatic hydrocarbon group which may have a substituent, in Formula (a1-r2-4), Ra′¹² and Ra′¹³ are each independently a monovalent chain saturated hydrocarbon group having 1 to 10 carbon atoms, some or all of the hydrogen atoms in the chain saturated hydrocarbon group may be substituted, Ra′¹⁴ is a hydrocarbon group which may have a substituent, and * indicates a bond (the same applies hereinafter)].

In Formula (a1-r2-1), Ra′¹⁰ is a linear or branched alkyl group having 1 to 12 carbon atoms, some of which may be substituted with a halogen atom or a heteroatom-containing group.

The linear alkyl group for Ra′¹⁰ has 1 to 12 carbon atoms, preferably has 1 to 10 carbon atoms, and particularly preferably has 1 to 5 carbon atoms.

Examples of branched alkyl groups for Ra′¹⁰ include the same groups as for Ra′³.

Some carbon atoms in the alkyl group for Ra′¹⁰ may be substituted with a halogen atom or a heteroatom-containing group. For example, some hydrogen atoms constituting the alkyl group may be substituted with a halogen atom or a heteroatom-containing group. In addition, some carbon atoms (a methylene group, etc.) constituting the alkyl group may be substituted with a heteroatom-containing group.

Examples of heteroatoms referred to here include an oxygen atom, a sulfur atom, and a nitrogen atom. Examples of heteroatom-containing groups include (—O—), —C(═O)—O—, —O—C(═O)—, —C(═O)—, —O—C(═O)—O—, —C(═O)—NH—, —NH—, —S—, —S(═O)₂—, and —S(═O)₂-O—.

In Formula (a1-r2-1), Ra′¹¹ (an aliphatic cyclic group formed together with a carbon atom to which Ra′¹⁰ is bonded) is preferably a group exemplified as an alicyclic hydrocarbon group (alicyclic hydrocarbon group) which is a monocyclic group or a polycyclic group for Ra′³ in Formula (a1-r-1). Among these, a monocyclic alicyclic hydrocarbon group is preferable, and specifically, a cyclopentyl group or a cyclohexyl group is more preferable.

In Formula (a1-r2-2), examples of cyclic hydrocarbon groups formed by Xa together with Ya include groups in which one or more hydrogen atoms have been additionally removed from a cyclic monovalent hydrocarbon group (alicyclic hydrocarbon group) for Ra′³ in Formula (a1-r-1).

The cyclic hydrocarbon group formed by Xa together with Ya may have a substituent. Examples of substituents include the same substituents that the cyclic hydrocarbon group for Ra′³ may have.

In Formula (a1-r2-2), examples of monovalent chain saturated hydrocarbon groups having 1 to 10 carbon atoms for Ra¹⁰¹ to Ra¹⁰³ include a methyl group, an ethyl group, a propyl group, a butyl group, a pentyl group, a hexyl group, a heptyl group, an octyl group, and a decyl group.

Examples of monovalent aliphatic cyclic saturated hydrocarbon groups having 3 to 20 carbon atoms for Ra¹⁰¹ to Ra¹⁰³ include monocyclic aliphatic saturated hydrocarbon groups such as a cyclopropyl group, a cyclobutyl group, a cyclopentyl group, a cyclohexyl group, a cycloheptyl group, a cyclooctyl group, a cyclodecyl group, and a cyclododecyl group; and polycyclic aliphatic saturated hydrocarbon groups such as a bicyclo[2.2.2]octanyl group, a tricyclo[5.2.1.02,6]decanyl group, a tricyclo[3.3.1.13,7]decanyl group, a tetracyclo[6.2.1.13,6.02,7]dodecanyl group, and an adamantyl group.

Among these, in consideration of ease of synthesis, Ra¹⁰¹ to Ra¹⁰³ are preferably a hydrogen atom or a monovalent chain saturated hydrocarbon group having 1 to 10 carbon atoms, and among these, a hydrogen atom, a methyl group, and an ethyl group are more preferable, and a hydrogen atom is particularly preferable.

Examples of substituents that the chain saturated hydrocarbon groups represented by Ra¹⁰¹ to Ra¹⁰³ or the aliphatic cyclic saturated hydrocarbon group have include the same groups as for Ra.

Examples of groups containing a carbon-carbon double bond generated when two or more of Ra¹⁰¹ to Ra¹⁰³ are bonded to each other to form a ring structure include a cyclopentenyl group, a cyclohexenyl group, a methylcyclopentenyl group, a methylcyclohexanyl group, a cyclopentylidenethenyl group, and a cyclohexylideneethenyl group. Among these, in consideration of ease of synthesis, a cyclopentenyl group, a cyclohexenyl group, or a cyclopentylidenethenyl group is preferable.

In Formula (a1-r2-3), the aliphatic cyclic group formed by Yaa together with Xaa is preferably a group exemplified as an alicyclic hydrocarbon group which is a monocyclic group or a polycyclic group for Ra′³ in Formula (a1-r-1).

In Formula (a1-r2-3), examples of aromatic hydrocarbon groups for Ra¹⁰⁴ include a group in which one or more hydrogen atoms have been removed from an aromatic hydrocarbon ring having 5 to 30 carbon atoms. Among these, Ra¹⁰⁴ is preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene or phenanthrene, still more preferably a group in which one or more hydrogen atoms have been removed from benzene, naphthalene or anthracene, particularly preferably a group in which one or more hydrogen atoms have been removed from benzene or naphthalene, and most preferably a group in which one or more hydrogen atoms have been removed from benzene.

Examples of substituents that Ra¹⁰⁴ in Formula (a1-r2-3) may have include a methyl group, an ethyl group, a propyl group, a hydroxyl group, a carboxyl group, a halogen atom, an alkoxy group (a methoxy group, an ethoxy group, a propoxy group, a butoxy group, etc.), and an alkyloxycarbonyl group.

In Formula (a1-r2-4), Ra′¹² and Ra′¹³ are each independently a monovalent chain saturated hydrocarbon group having 1 to 10 carbon atoms. Examples of monovalent chain saturated hydrocarbon groups having 1 to 10 carbon atoms for Ra′¹² and Ra′¹³ include the same monovalent chain saturated hydrocarbon groups having 1 to 10 carbon atoms for Ra¹⁰¹ to Ra¹⁰³. Some or all of the hydrogen atoms in the chain saturated hydrocarbon group may be substituted.

Among these, Ra′¹² and Ra′¹³ are preferably an alkyl group having 1 to 5 carbon atoms, more preferably an alkyl group having 1 to 5 carbon atoms, still more preferably a methyl group or an ethyl group, and particularly preferably a methyl group.

When the chain saturated hydrocarbon groups represented by Ra′¹² and Ra′¹³ are substituted, examples of substituents include the same groups as for Ra^{x5}.

In Formula (a1-r2-4), Ra′¹⁴ is a hydrocarbon group which may have a substituent. Examples of hydrocarbon groups for Ra′¹⁴ include a linear or branched alkyl group and a cyclic hydrocarbon group.

The linear alkyl group for Ra′¹⁴ is preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group or an n-butyl group is preferable, and a methyl group or an ethyl group is more preferable.

The branched alkyl group for Ra′¹⁴ is preferably 3 to 10 carbon atoms and more preferably 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group, and an isopropyl group is preferable.

When Ra′¹⁴ is a cyclic hydrocarbon group, the hydrocarbon group may be an alicyclic hydrocarbon group or an aromatic hydrocarbon group, and may be a polycyclic group or a monocyclic group.

The alicyclic hydrocarbon group which is a monocyclic group is preferably a group in which one hydrogen atom has been removed from a monocycloalkane. The monocycloalkane preferably has 3 to 6 carbon atoms, and specific examples thereof include cyclopentane and cyclohexane.

The alicyclic hydrocarbon group which is a polycyclic group is preferably a group in which one hydrogen atom has been removed from a polycycloalkane, and the polycycloalkane preferably has 7 to 12 carbon atoms, and specific examples thereof include adamantane, norbornane, isobornane, tricyclodecane, and tetracyclododecane.

Examples of aromatic hydrocarbon groups for Ra′¹⁴ include the same aromatic hydrocarbon groups as for Ra¹⁰⁴. Among these, Ra′¹⁴ is preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene or phenanthrene, still more preferably a group in which one or more hydrogen atoms have been removed from benzene, naphthalene or anthracene, particularly preferably a group in which one or more hydrogen atoms have been removed from naphthalene or anthracene, and most preferably a group in which one or more hydrogen atoms have been removed from naphthalene.

Examples of substituents that Ra′¹⁴ may have include the same substituents that Ra¹⁰⁴ may have.

When Ra′¹⁴ in Formula (a1-r2-4) is a naphthyl group, the position at which it is bonded to the tertiary carbon atom in Formula (a1-r2-4) may be either the 1^{st} or 2^{nd} position of the naphthyl group.

When Ra′¹⁴ in Formula (a1-r2-4) is an anthryl group, the position at which it is bonded to the tertiary carbon atom in Formula (a1-r2-4) may be any of the 1^{st}, 2^{nd}, and 9^{th} positions of the anthryl group.

Specific examples of groups represented by Formula (a1-r2-1) are shown below.

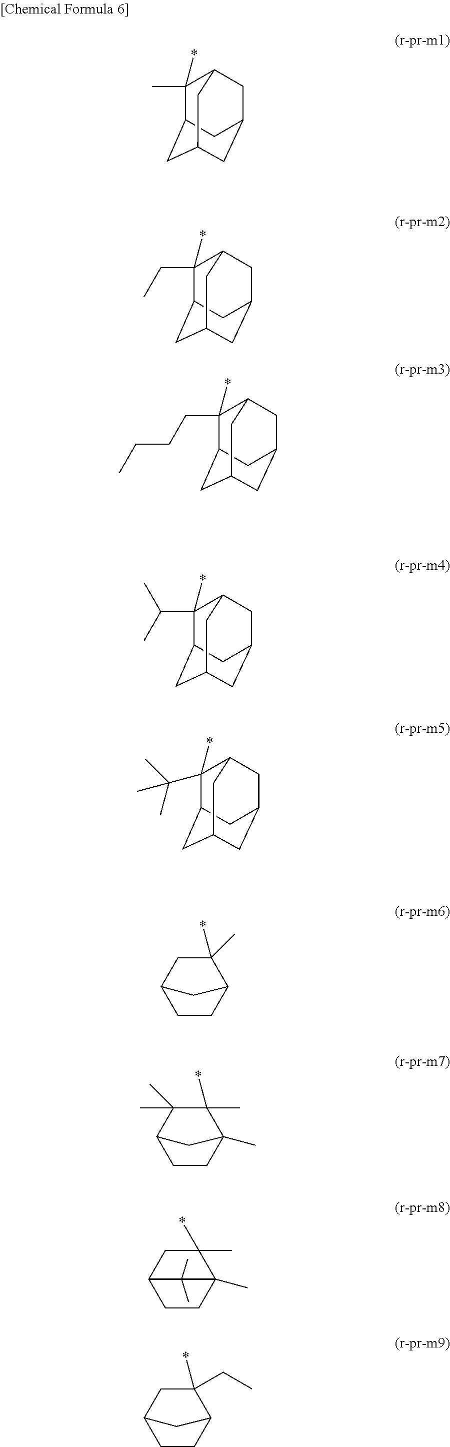

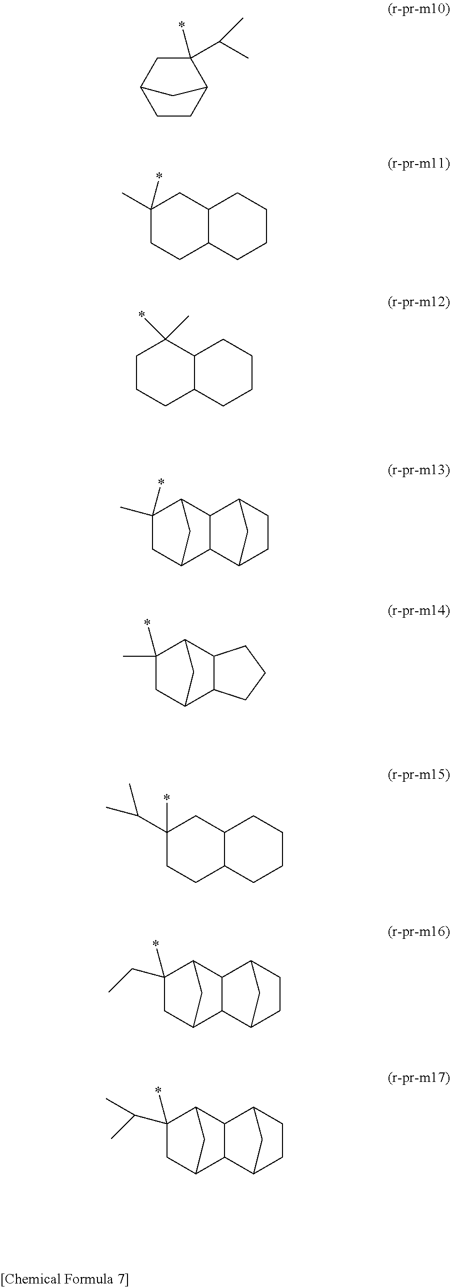

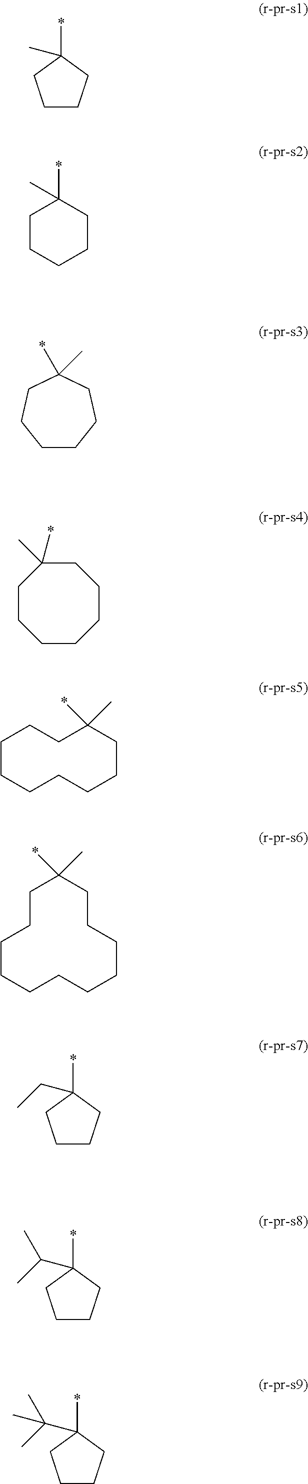

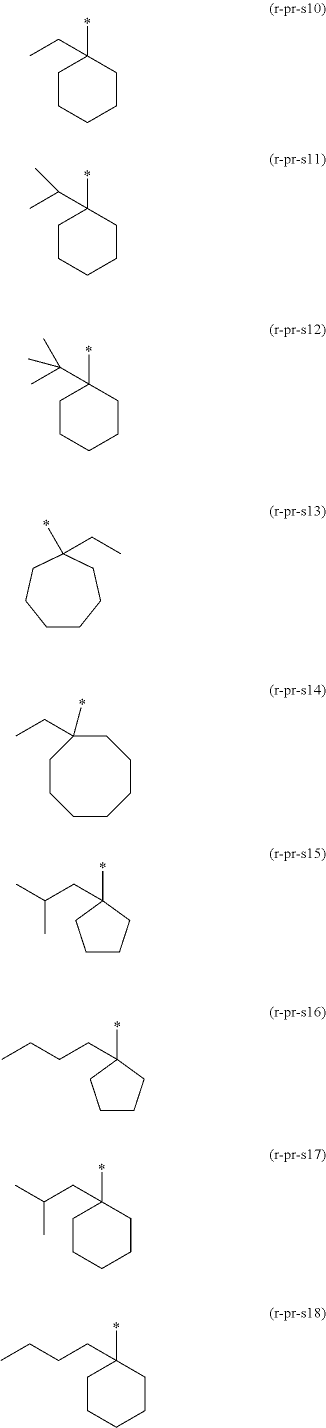

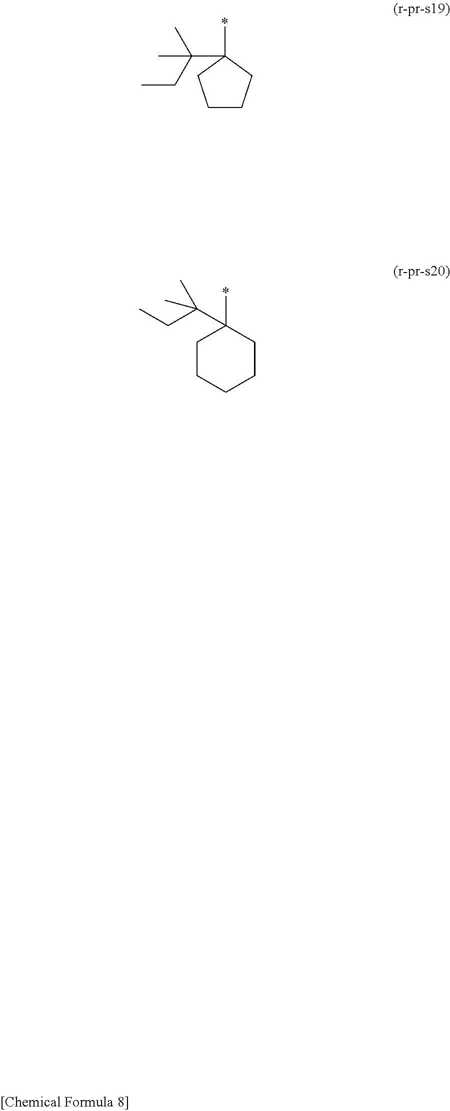 [SMILES: *C1(C(C)(C)CC)CCCC1 ; *C1(C(C)(C)CC)CCCCC1]

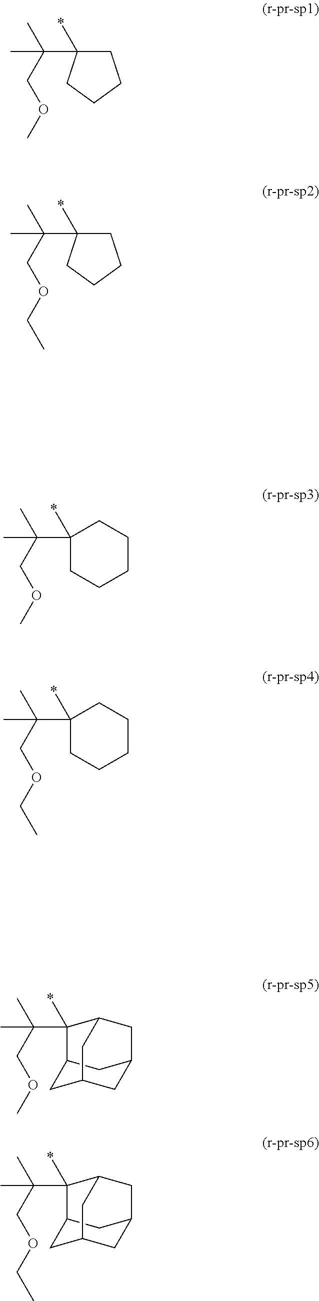

Specific examples of groups represented by Formula (a1-r2-2) are shown below.

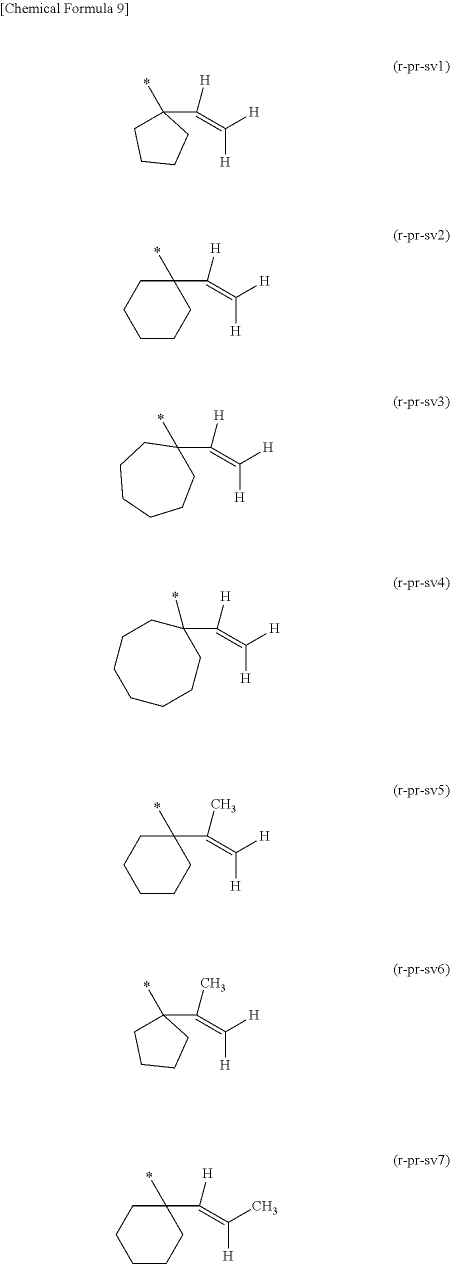

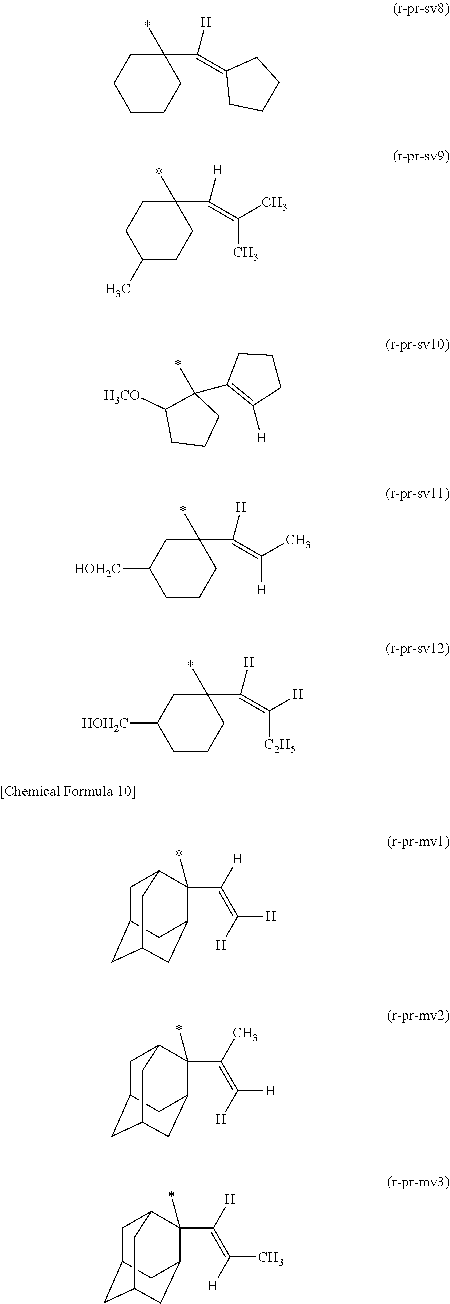

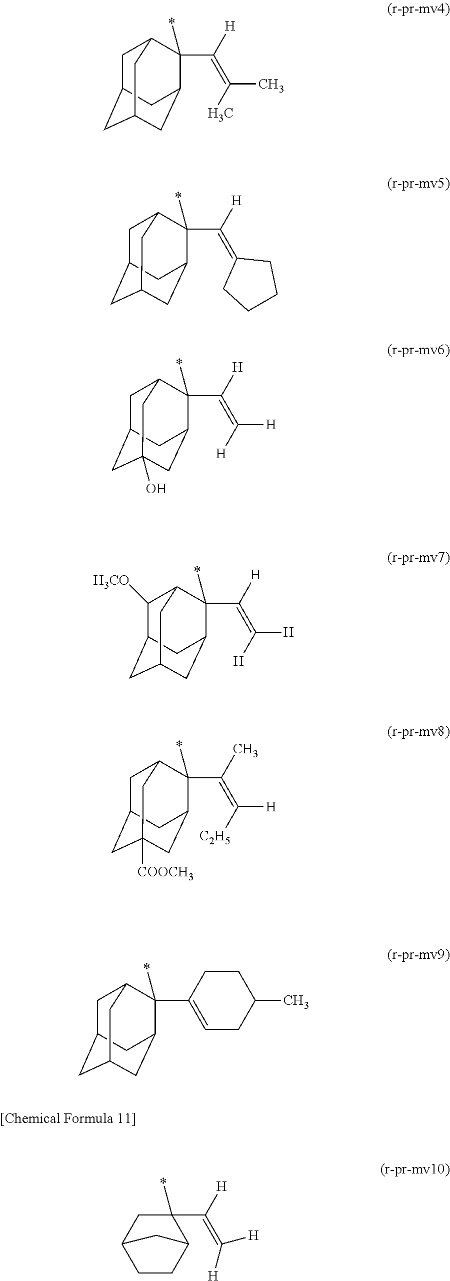

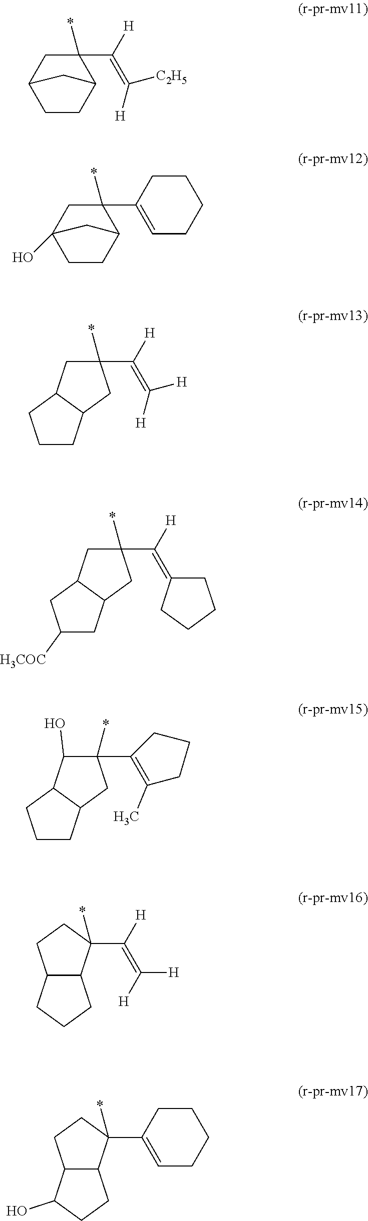

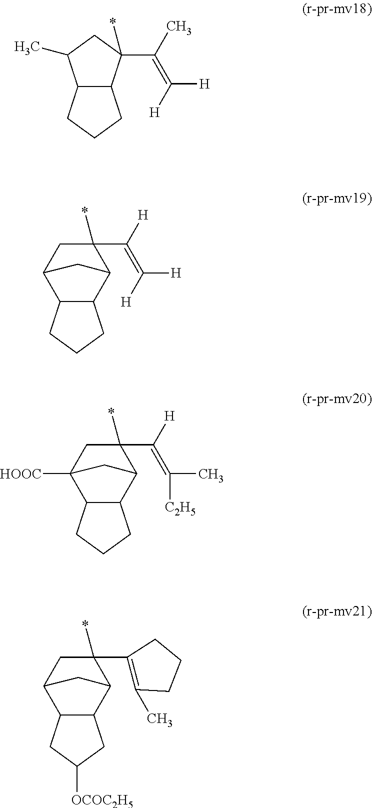

Specific examples of groups represented by Formula (a1-r2-3) are shown below.

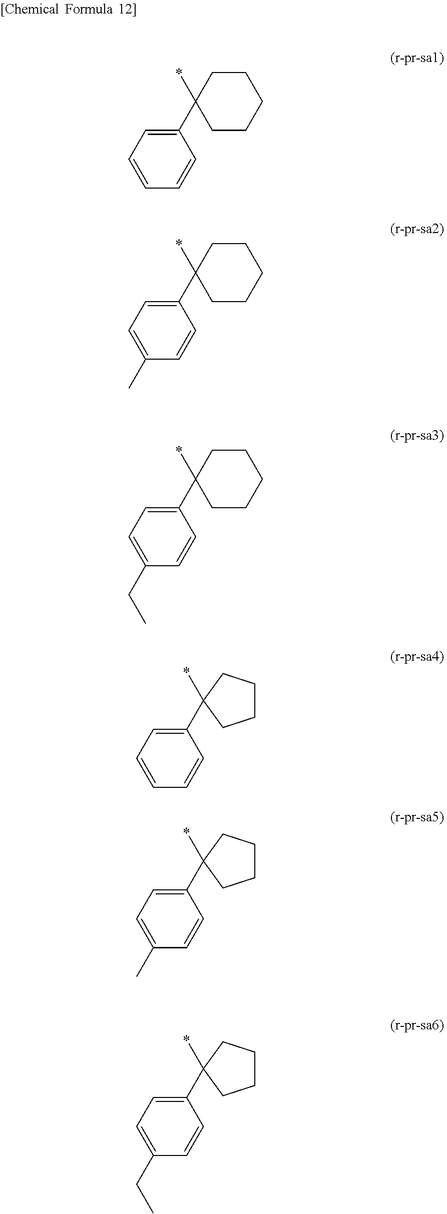

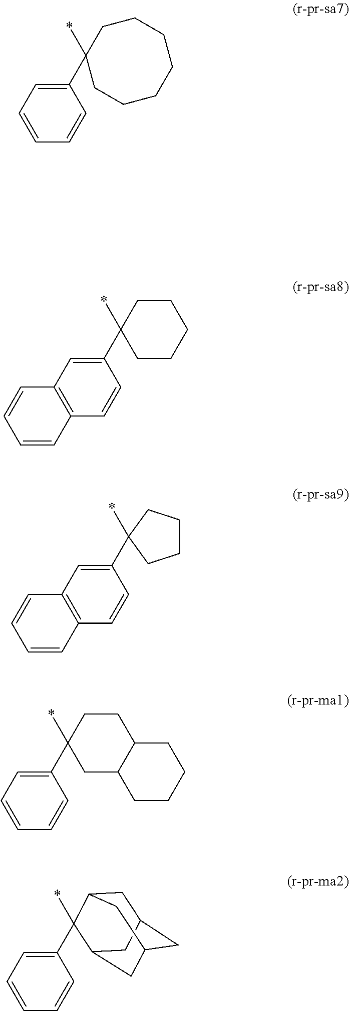 [SMILES: *C1(c2ccccc2)CCCCCCC1 ; *C1(c2ccc3ccccc3c2)CCCCC1 ; *C1(c2ccc3ccccc3c2)CCCC1 ; *C1(c2ccccc2)CCC2CCCCC2C1 ; *C1(c2ccccc2)C2CC3CC(C2)CC1C3]

Specific examples of groups represented by Formula (a1-r2-4) are shown below.

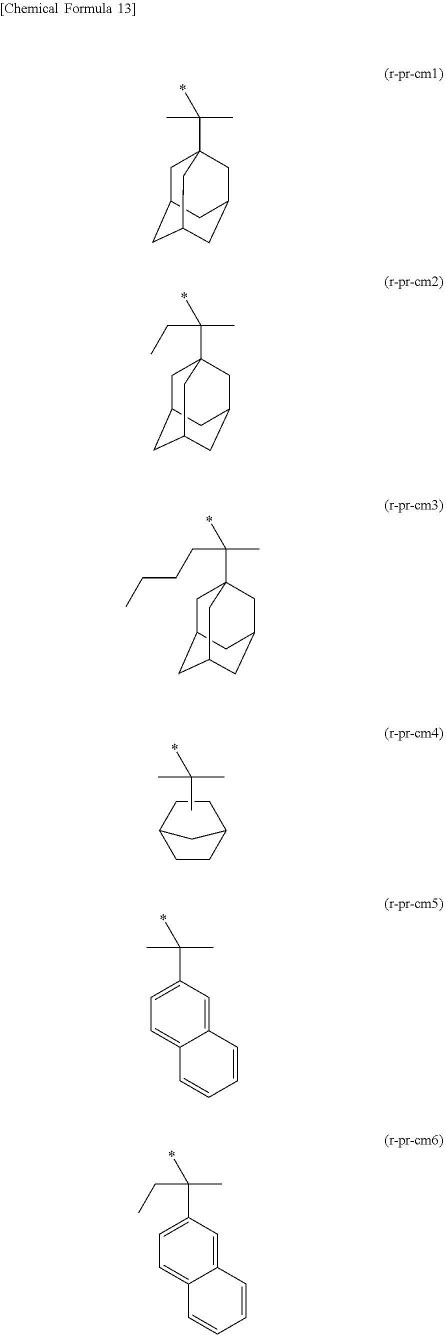

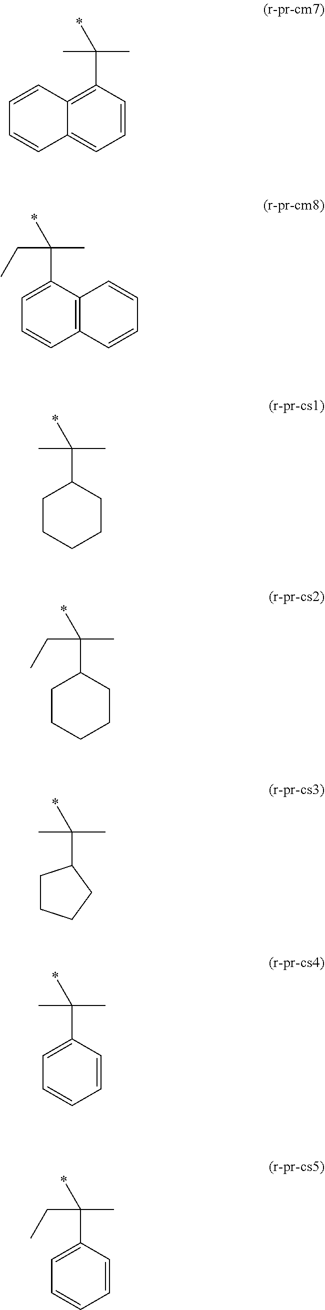

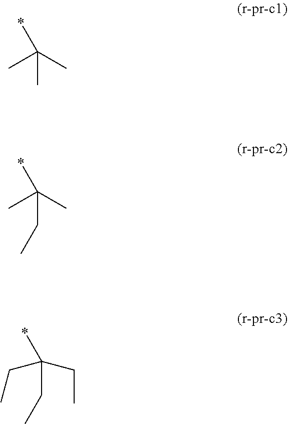

### Tertiary Alkyloxycarbonyl Acid-Dissociable Group:

Examples of acid-dissociable groups that protect a hydroxyl group among the polar groups include an acid-dissociable group represented by the following General Formula (a1-r-3) (will sometimes be referred to hereinafter as a “tertiary alkyloxycarbonyl acid-dissociable group” for convenience).

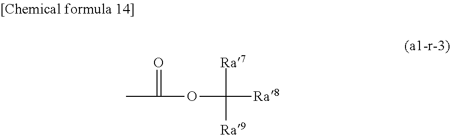

[in the formula, Ra′⁷ to Ra′⁹ are each an alkyl group].

In Formula (a1-r-3), Ra′⁷ to Ra′⁹ are each preferably an alkyl group having 1 to 5 carbon atoms, and more preferably an alkyl group having 1 to 3 carbon atoms.

In addition, each alkyl group preferably has a total number of carbon atoms of 3 to 7, more preferably has 3 to 5 carbon atoms, and most preferably has 3 and 4 carbon atoms.

### Secondary Alkyl Ester Type Acid-Dissociable Group:

Examples of acid-dissociable groups that protect a carboxy group among the above polar groups include an acid-dissociable group represented by the following General Formula (a1-r-4).

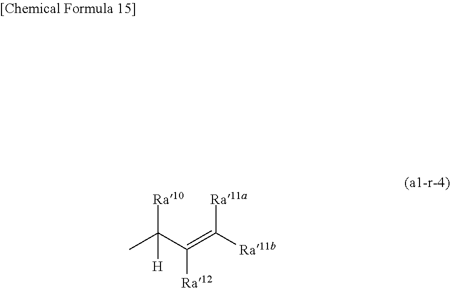

[in the formula, Ra′¹⁰ is a hydrocarbon group, Ra′^{11a} and Ra′^{11b} are each independently a hydrogen atom, a halogen atom or an alkyl group, Ra′¹² is a hydrogen atom or a hydrocarbon group, Ra′¹⁰ and Ra′^{11a} or Ra′^{11b} may be bonded to each other to form a ring, and Ra′^{11a} or Ra′^{11b} and Ra′¹² may be bonded to each other to form a ring].

In the formula, examples of hydrocarbon groups for Ra′¹⁰ and Ra′¹² include the same groups as for Ra′³.

In the formula, examples of alkyl groups for Ra′^{11a} and Ra′^{11b} include the same alkyl groups as for Ra′¹.

In the formula, the hydrocarbon groups for Ra′¹⁰ and Ra′¹² and the alkyl groups for Ra′^{11a} and Ra′^{11b} may have a substituent. Examples of substituents include those exemplified for Ra.

Ra′¹⁰ and Ra′^{11a} or Ra′^{11b} may be bonded to each other to form a ring. The ring may be a polycycle, a monocycle, an alicycle, or an aromatic ring.

The alicycle and aromatic ring may contain a heteroatom.

As the ring formed by bonding Ra′¹⁰ and Ra′^{11a} or Ra′^{11b} among the above examples, a monocycloalkene, a ring in which some carbon atoms in a monocycloalkene are substituted with a heteroatom (an oxygen atom, a sulfur atom, etc.), and a monocycloalkadiene are preferable, a cycloalkene having 3 to 6 carbon atoms is preferable, and a cyclopentene or a cyclohexene is preferable.

The ring formed by bonding Ra′¹⁰ and Ra′^{11a} or Ra′^{11b} may be a fused ring. Specific examples of fused rings include indane.

The ring formed by bonding Ra′¹⁰ and Ra′^{11a} or Ra′^{11b} may have a substituent. Examples of substituents include those exemplified for Ra.

Ra′^{11a} or Ra′^{11b} and Ra′¹² may be bonded to each other to form a ring, and examples of rings include the same rings formed by bonding Ra′¹⁰ and Ra′^{11a} or Ra′^{11b}.

Specific examples of groups represented by Formula (a1-r-4) are shown below.

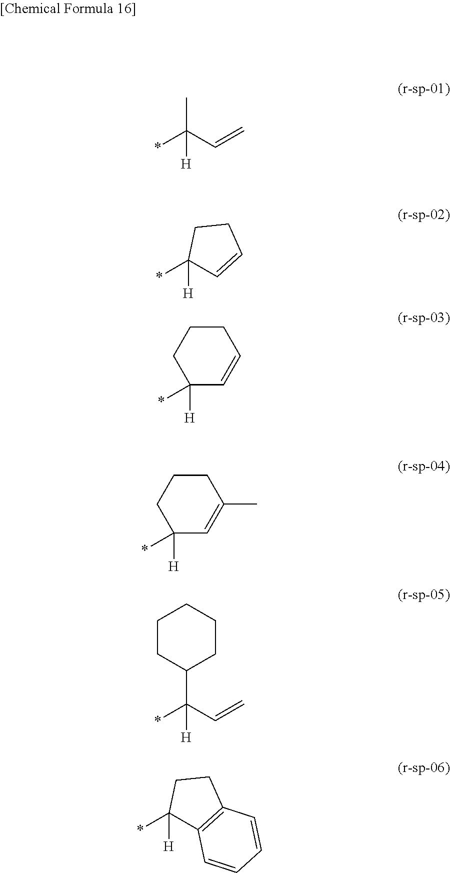

Examples of structural units (a1) include a structural unit derived from an acrylic acid ester in which the hydrogen atom bonded to the carbon atom at the α-position may be substituted with a substituent, a structural unit derived from an acrylamide, a structural unit in which at least some hydrogen atoms in the hydroxyl group of a structural unit derived from hydroxystyrene or a hydroxystyrene derivative are protected by a substituent containing the acid-decomposable group, and a structural unit in which at least some hydrogen atoms in —C(═O)—OH of a structural unit derived from vinylbenzoic acid or a vinylbenzoic acid derivative are protected by a substituent containing the acid-decomposable group.

Among the above examples, the structural unit (a1) is preferably a structural unit derived from an acrylic acid ester in which the hydrogen atom bonded to the carbon atom at the α-position may be substituted with a substituent.

Preferable specific examples of such structural units (a1) include structural units represented by the following General Formula (a1-1) or (a1-2).

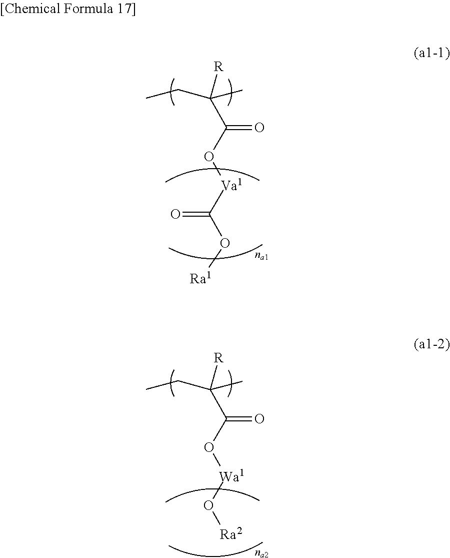

[in the formula, R is a hydrogen atom, an alkyl group having 1 to 5 carbon atoms or a halogenated alkyl group having 1 to 5 carbon atoms, Va¹ is a divalent hydrocarbon group which may have an ether bond, nₐ₁ is an integer of 0 to 2, Ra¹ is the acid-dissociable group represented by General Formula (a1-r-1) or (a1-r-2), Wa¹ is an nₐ₂+monovalent hydrocarbon group, nₐ₂ is an integer of 1 to 3, and Ra² is the acid-dissociable group represented by General Formula (a1-r-1) or (a1-r-3)].

In Formula (a1-1), the alkyl group having 1 to 5 carbon atoms for R is preferably a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which some or all of the hydrogen atoms in the alkyl group having 1 to 5 carbon atoms are substituted with halogen atoms. The halogen atom is particularly preferably a fluorine atom.

R is preferably a hydrogen atom, an alkyl group having 1 to 5 carbon atoms or a fluorinated alkyl group having 1 to 5 carbon atoms, and in terms of industrial availability, a hydrogen atom or a methyl group is most preferable.

In Formula (a1-1), the divalent hydrocarbon group for Va¹ may be an aliphatic hydrocarbon group or an aromatic hydrocarbon group.

The aliphatic hydrocarbon group as the divalent hydrocarbon group for Va¹ may be saturated or unsaturated, and is generally preferably saturated.

More specific examples of aliphatic hydrocarbon groups include a linear or branched aliphatic hydrocarbon group and an aliphatic hydrocarbon group containing a ring in the structure.

The linear aliphatic hydrocarbon group preferably has 1 to 10 carbon atoms, more preferably has 1 to 6 carbon atoms, still more preferably has 1 to 4 carbon atoms, and most preferably has 1 to 3 carbon atoms.

The linear aliphatic hydrocarbon group is preferably a linear alkylene group, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group preferably has 2 to 10 carbon atoms, more preferably has 3 to 6 carbon atoms, still more preferably has 3 or 4 carbon atoms, and most preferably has 3 carbon atoms.

The branched aliphatic hydrocarbon group is preferably a branched alkylene group, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. The alkyl group in the alkylalkylene group is preferably a linear alkyl group having 1 to 5 carbon atoms.

Examples of aliphatic hydrocarbon groups containing a ring in the structure include an alicyclic hydrocarbon group (a group in which two hydrogen atoms have been removed from an aliphatic hydrocarbon ring), a group in which an alicyclic hydrocarbon group is bonded to the end of a linear or branched aliphatic hydrocarbon group, and a group in which an alicyclic hydrocarbon group is inserted into a linear or branched aliphatic hydrocarbon group. Examples of linear or branched aliphatic hydrocarbon groups include the same linear aliphatic hydrocarbon groups or branched aliphatic hydrocarbon groups.

The alicyclic hydrocarbon group preferably has 3 to 20 carbon atoms and more preferably has 3 to 12 carbon atoms.

The alicyclic hydrocarbon group may be polycyclic or monocyclic. The monocyclic alicyclic hydrocarbon group is preferably a group in which two hydrogen atoms have been removed from a monocycloalkane. The monocycloalkane preferably has 3 to 6 carbon atoms, and specific examples thereof include cyclopentane and cyclohexane. The polycyclic alicyclic hydrocarbon group is preferably a group in which two hydrogen atoms have been removed from a polycycloalkane, the polycycloalkane preferably has 7 to 12 carbon atoms, and specific examples thereof include adamantane, norbornane, isobornane, tricyclodecane, and tetracyclododecane.

The aromatic hydrocarbon group as the divalent hydrocarbon group for Va¹ is a hydrocarbon group having an aromatic ring.

The aromatic hydrocarbon group preferably has 3 to 30 carbon atoms, more preferably has 5 to 30 carbon atoms, still more preferably has 5 to 20 carbon atoms, particularly preferably has 6 to 15 carbon atoms, and most preferably has 6 to 12 carbon atoms. Here, the number of carbon atoms does not include the number of carbon atoms in the substituent.

Specific examples of aromatic rings in the aromatic hydrocarbon group include aromatic hydrocarbon rings such as benzene, biphenyl, fluorene, naphthalene, anthracene, and phenanthrene; and aromatic heterocycles in which some carbon atoms constituting the aromatic hydrocarbon ring are substituted with heteroatoms. Examples of heteroatoms in aromatic heterocycles include an oxygen atom, a sulfur atom, and a nitrogen atom.

Specific examples of aromatic hydrocarbon groups include a group in which two hydrogen atoms have been removed from the aromatic hydrocarbon ring (arylene group); and a group in which one hydrogen atom of a group (aryl group) in which one hydrogen atom has been removed from the aromatic hydrocarbon ring is substituted with an alkylene group (for example, a group in which one hydrogen atom has been additionally removed from the aryl group in the arylalkyl group such as a benzyl group, a phenethyl group, a 1-naphthyl methyl group, a 2-naphthyl methyl group, a 1-naphthyl ethyl group, and a 2-naphthyl ethyl group). The alkylene group (the alkyl chain in the arylalkyl group) preferably has 1 to 4 carbon atoms, more preferably has 1 to 2 carbon atoms, and particularly preferably has 1 carbon atom.

In Formula (a1-1), Ra¹ is the acid-dissociable group represented by Formula (a1-r-1) or (a1-r-2).

In Formula (a1-2), the nₐ₂+monovalent hydrocarbon group for Wa¹ may be an aliphatic hydrocarbon group or an aromatic hydrocarbon group. The aliphatic hydrocarbon group is a hydrocarbon group having no aromaticity, and may be saturated or unsaturated, and is generally preferably saturated. Examples of aliphatic hydrocarbon groups include a linear or branched aliphatic hydrocarbon group, an aliphatic hydrocarbon group containing a ring in the structure, and a group in which a linear or branched aliphatic hydrocarbon group and an aliphatic hydrocarbon group containing a ring in the structure are combined.

The valence of nₐ₂+1 is preferably 2 to 4 and more preferably 2 or 3.

In Formula (a1-2), Ra² is the acid-dissociable group represented by General Formula (a1-r-1) or (a1-r-3).

Specific examples of structural units (a1) are shown below. In the following formulae, R^{α} is a hydrogen atom, a methyl group or a trifluoromethyl group.

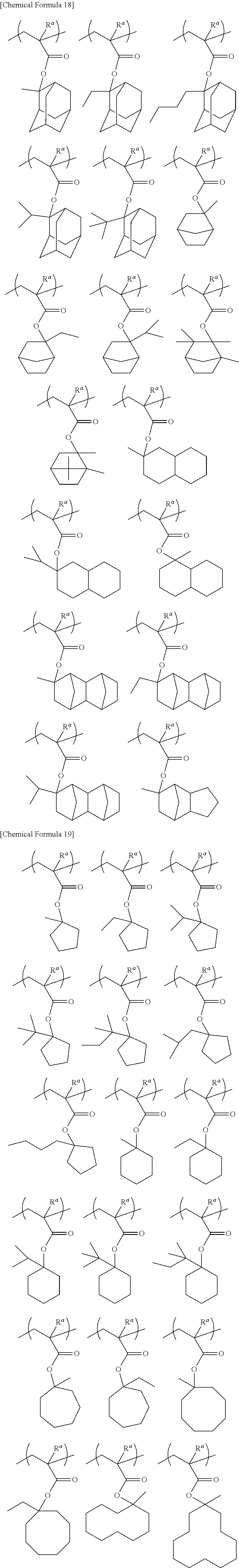

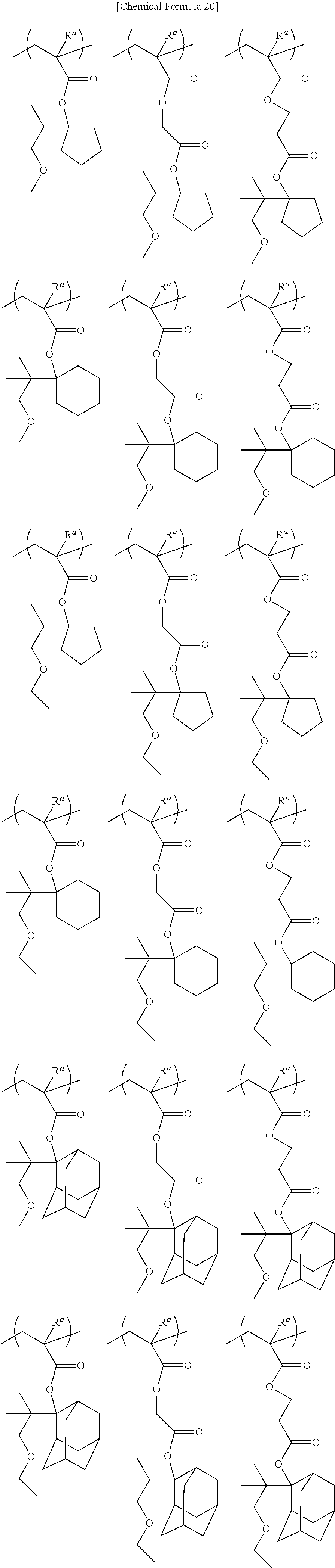

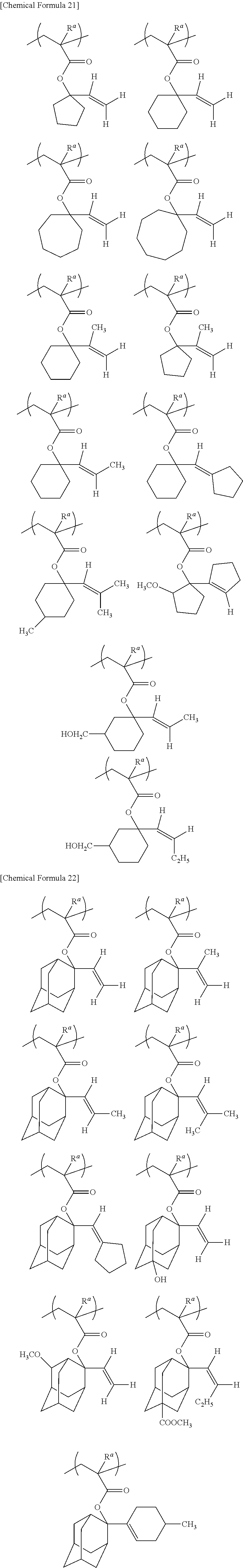

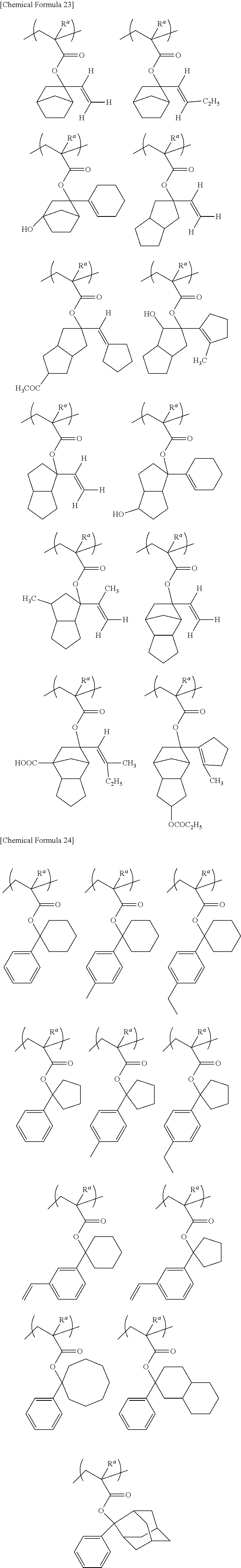

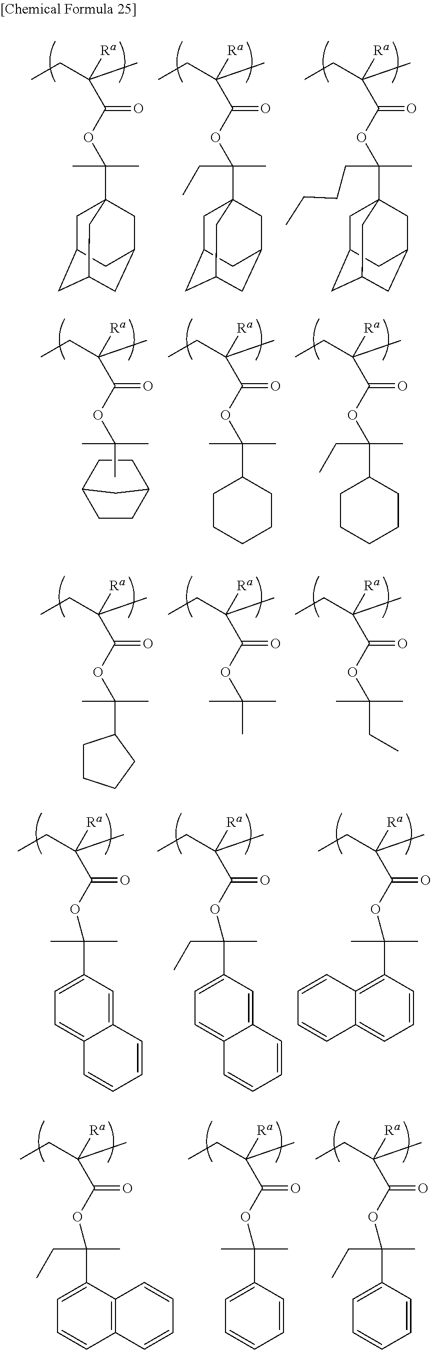

The structural unit (a1) of the component (A1) may be of one type or of two or more types.

As the structural unit (a1), the structural unit represented by Formula (a1-1) is more preferable because it is easier to further improve properties (CDU, etc.) in electron beam or EUV lithography.

Among these, the structural unit (a1) is particularly preferably a structural unit represented by the following General Formula (a1-1-1).

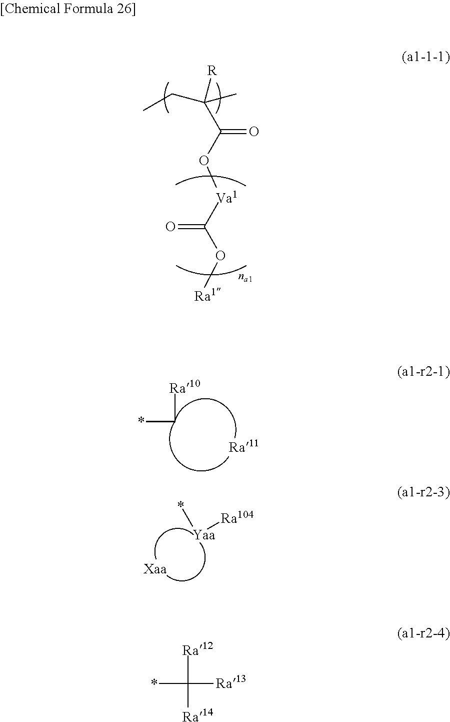

[in the formula, Ra^{1″} is an acid-dissociable group represented by General Formula (a1-r2-1), (a1-r2-3) or (a1-r2-4), and * indicates a bond].

In Formula (a1-1-1), R, Va¹ and nₐ₁ are the same as R, Va¹ and nₐ₁ in Formula (a1-1).

The acid-dissociable group represented by General Formula (a1-r2-1), (a1-r2-3) or (a1-r2-4) is as described above. Among these, it is preferable to select an acid-dissociable group that is a cyclic group because it is suitable to improve the reactivity with EB or EUV, and an acid-dissociable group represented by General Formula (a1-r2-1) is more preferable.

The proportion of the structural unit (a1) in the component (A1) based on a total amount (100 mol %) of all structural units constituting the component (A1) is preferably 5 to 95 mol %, more preferably 10 to 90 mol %, still more preferably 30 to 70 mol %, and particularly preferably 40 to 65 mol %.

When the proportion of the structural unit (a1) is set to be equal to or larger than the lower limit value of the preferable range, lithography properties such as the sensitivity, CDU, resolution, and roughness are improved. On the other hand, when the proportion is set to be equal to or smaller than the upper limit value of the preferable range, a balance with other structural units can be achieved and various lithography properties become favorable.

### <<Other Structural Units>>

The component (A1) may have, as necessary, other structural units, in addition to the above structural unit (a1).

Examples of other structural units include a structural unit (a10) represented by the following General Formula (a10-1); a structural unit (a2) having a lactone-containing cyclic group; and a structural unit (a8) derived from a compound represented by the following General Formula (a8-1).

Structural Unit (a10):

The structural unit (a10) is a structural unit represented by the following General Formula (a10-1) (excluding those that correspond to the structural unit (a1)).

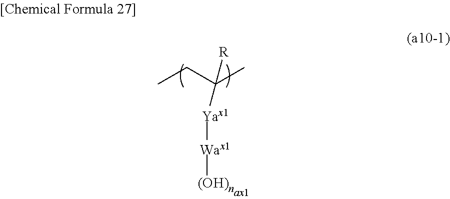

[in the formula. R is a hydrogen atom, an alkyl group having 1 to 5 carbon atoms or a halogenated alkyl group having 1 to 5 carbon atoms, Ya^{x1} is a single bond or a divalent linking group, Wa^{x1} is an aromatic hydrocarbon group which may have a substituent, and nₐₓ₁ is an integer of 1 or more].

In Formula (a10-1), R is a hydrogen atom, an alkyl group having 1 to 5 carbon atoms or a halogenated alkyl group having 1 to 5 carbon atoms.

R is preferably a hydrogen atom, an alkyl group having 1 to 5 carbon atoms or a fluorinated alkyl group having 1 to 5 carbon atoms, and in terms of industrial availability, a hydrogen atom, a methyl group or a trifluoromethyl group is more preferable, a hydrogen atom or a methyl group is still more preferable, and a hydrogen atom is particularly preferable.

In Formula (a10-1), Ya^{x1} is a single bond or a divalent linking group.

In the chemical formula, the divalent linking group for Ya^{x1} is not particularly limited, and preferable examples thereof include a divalent hydrocarbon group which may have a substituent and a heteroatom-containing divalent linking group.

Ya^{x1} is preferably a single bond, an ester bond [—C(═O)—O—, —O—C(═O)—], an ether bond (—O—), a linear or branched alkylene group, or a combination thereof, and more preferably a single bond or an ester bond [—C(═O)—O—, —O—C(═O)—].

In Formula (a10-1), Wa^{x1} is an aromatic hydrocarbon group which may have a substituent.

Examples of aromatic hydrocarbon groups for Wa^{x1} include a group in which (nₐₓ₁+1) hydrogen atoms have been removed from an aromatic ring which may have a substituent. Here, the aromatic ring is not particularly limited as long as it is a cyclic conjugated system having 4n+2 π electrons. The aromatic ring preferably has 5 to 30 carbon atoms, more preferably has 5 to 20 carbon atoms, still more preferably has 6 to 15 carbon atoms, and particularly preferably 6 to 12 carbon atoms. Specific examples of aromatic rings include aromatic hydrocarbon rings such as benzene, naphthalene, anthracene, and phenanthrene; and aromatic heterocycles in which some carbon atoms constituting the aromatic hydrocarbon ring are substituted with heteroatoms. Examples of heteroatoms in aromatic heterocycles include an oxygen atom, a sulfur atom, and a nitrogen atom. Specific examples of aromatic heterocycles include a pyridine ring and a thiophene ring.

In addition, examples of aromatic hydrocarbon groups for Wa^{x1} include a group in which (nₐₓ₁+1) hydrogen atoms have been removed from an aromatic compound (for example, biphenyl, fluorine, etc.) containing an aromatic ring which may have two or more substituents.

Among the above examples, Wa^{x1} is preferably a group in which (nₐₓ₁+1) hydrogen atoms have been removed from benzene, naphthalene, anthracene or biphenyl, more preferably a group in which (nₐₓ₁+1) hydrogen atoms have been removed from benzene or naphthalene, and still more preferably a group in which (nₐₓ₁+1) hydrogen atoms have been removed from benzene.

The aromatic hydrocarbon group for Wa^{x1} may or may not have a substituent. Examples of substituents include an alkyl group, an alkoxy group, a halogen atom, and a halogenated alkyl group. Examples of alkyl groups, alkoxy groups, halogen atoms, and halogenated alkyl groups as the substituents include the same as those exemplified as the substituents for the cyclic alicyclic hydrocarbon groups for Ya^{x1}. The substituent is preferably a linear or branched alkyl group having 1 to 5 carbon atoms, more preferably a linear or branched alkyl group having 1 to 3 carbon atoms, still more preferably an ethyl group or a methyl group, and particularly preferably a methyl group. The aromatic hydrocarbon group for Wa^{x1} preferably has no substituent.

In Formula (a10-1), nₐₓ₁ is an integer of 1 or more, is preferably an integer of 1 to 10, more preferably an integer of 1 to 5, still more preferably 1, 2 or 3, and particularly preferably 1 or 2.

Specific examples of structural units (a10) represented by Formula (a10-1) are shown below.

In the following formulae, R^{α} is a hydrogen atom, a methyl group or a trifluoromethyl group.

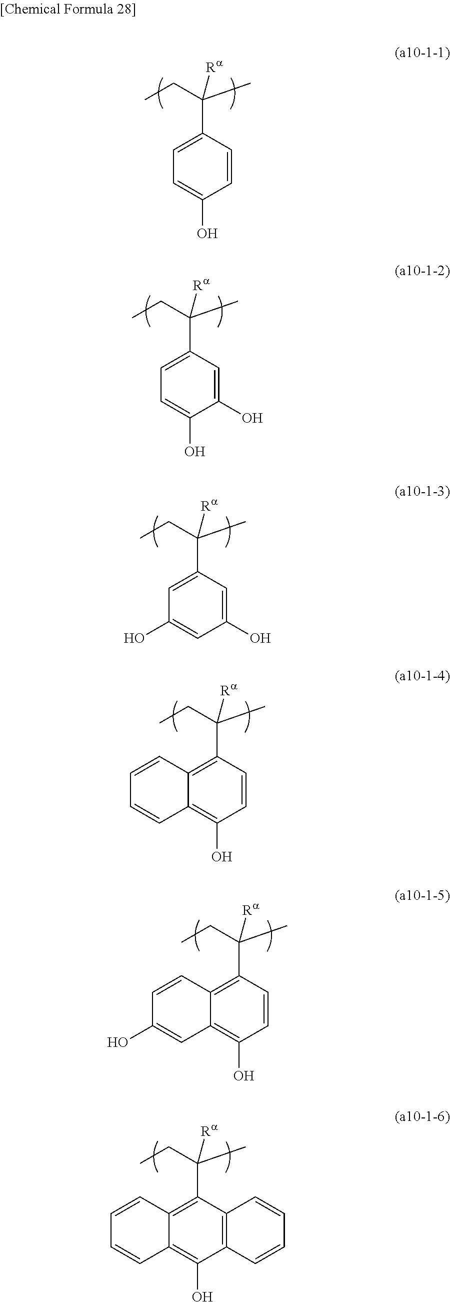

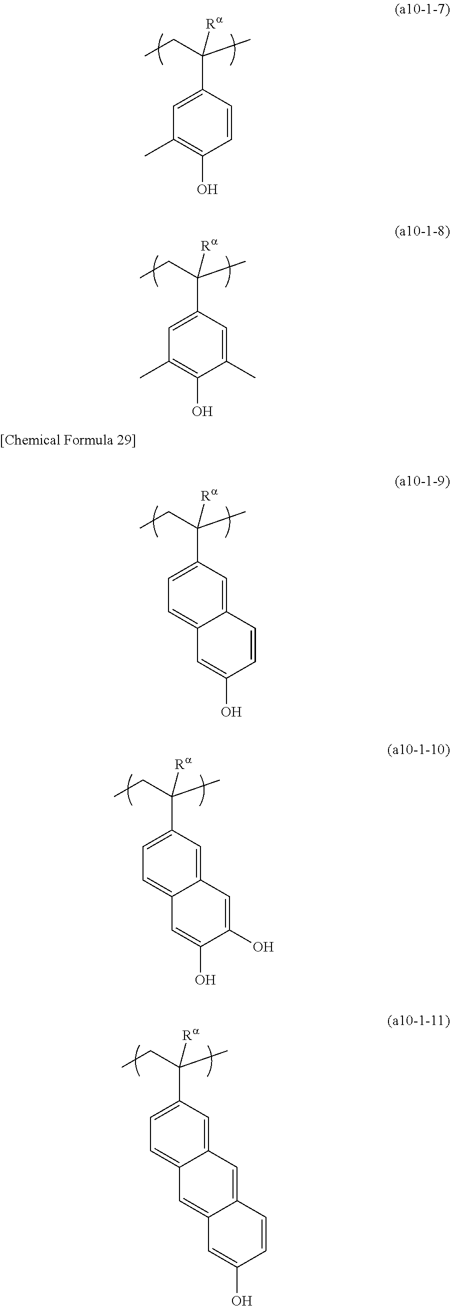

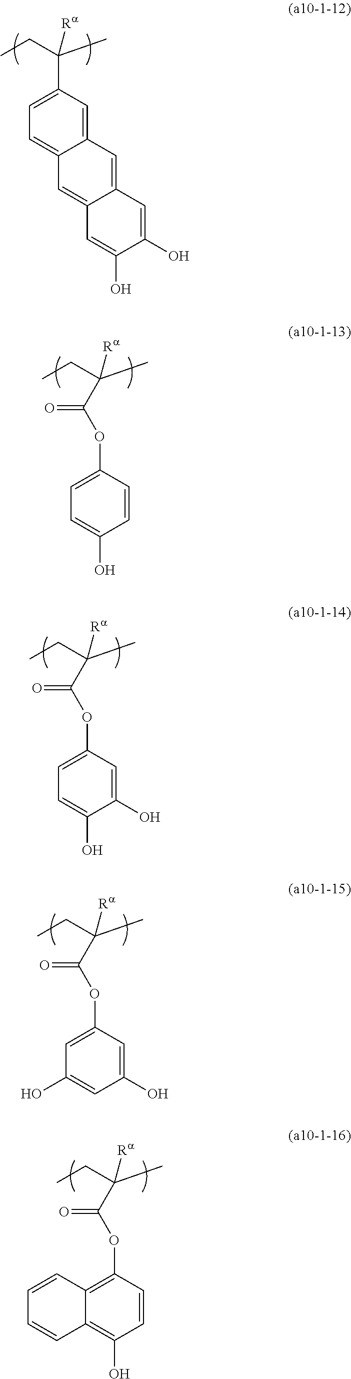

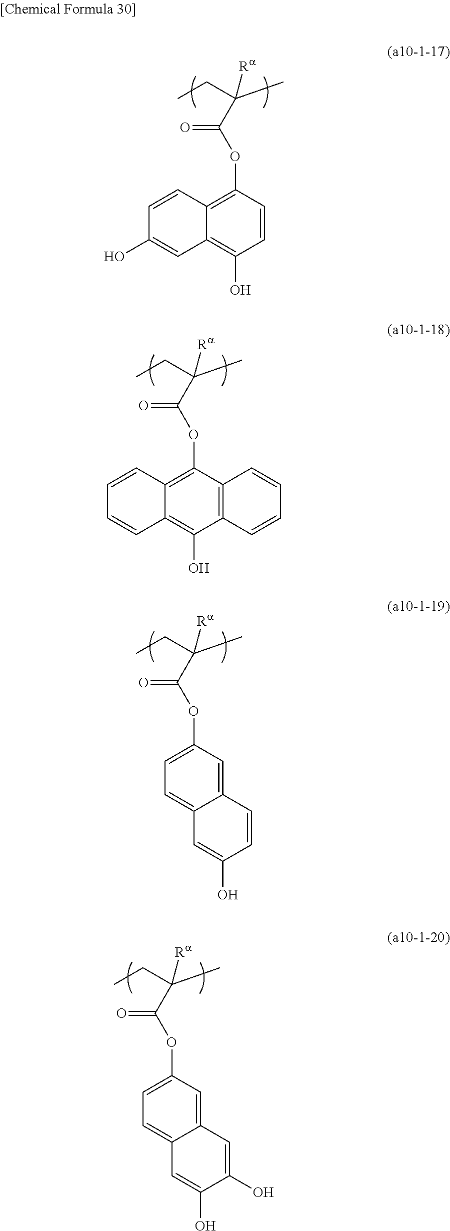

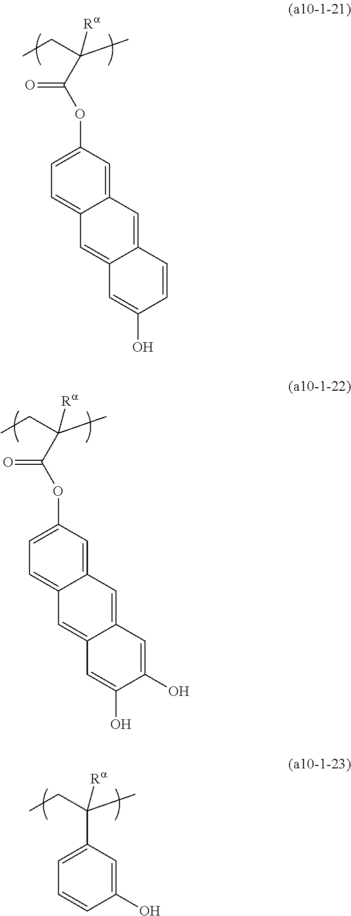

The structural unit (a10) of the component (A1) may be of one type or of two or more types.

When the component (A1) has the structural unit (a10), the proportion of the structural unit (a10) in the component (A1) based on a total amount (100 mol %) of all structural units constituting the component (A1) is preferably 20 to 80 mol %, more preferably 30 to 70 mol %, and still more preferably 30 to 60 mol %.

When the proportion of the structural unit (a10) is set to be equal to or larger than the lower limit value, it is easier to further improve the sensitivity. On the other hand, when the proportion is set to be equal to or smaller than the upper limit value, it becomes easier to achieve a balance with other structural units.

### Structural Unit (a2):

The component (A1) may further have a structural unit (a2) having a lactone-containing cyclic group (excluding those that correspond to the structural unit (a1)).

When the component (A1) is used to form a resist film, the lactone-containing cyclic group in the structural unit (a2) is effective in improving the adhesion of the resist film to the substrate. In addition, when the structural unit (a2) is provided, for example, effects of appropriately adjusting the acid diffusion length, improving the adhesion of the resist film to the substrate, and appropriately adjusting the solubility during development are obtained, resulting in improved lithography properties and the like.

The term “lactone-containing cyclic group” refers to a cyclic group containing a ring containing —O—C(═O)— (lactone ring) in its ring framework. The lactone ring is counted as the first ring, and when there is only a lactone ring, it is called a monocyclic group, and when there are additional other ring structures, it is called a polycyclic group regardless of the structure. The lactone-containing cyclic group may be a monocyclic group or a polycyclic group.

The lactone-containing cyclic group in the structural unit (a2) is not particularly limited, and any lactone-containing cyclic group can be used. Specific examples thereof include groups represented by the following General Formulae (a2-r-1) to (a2-r-7).

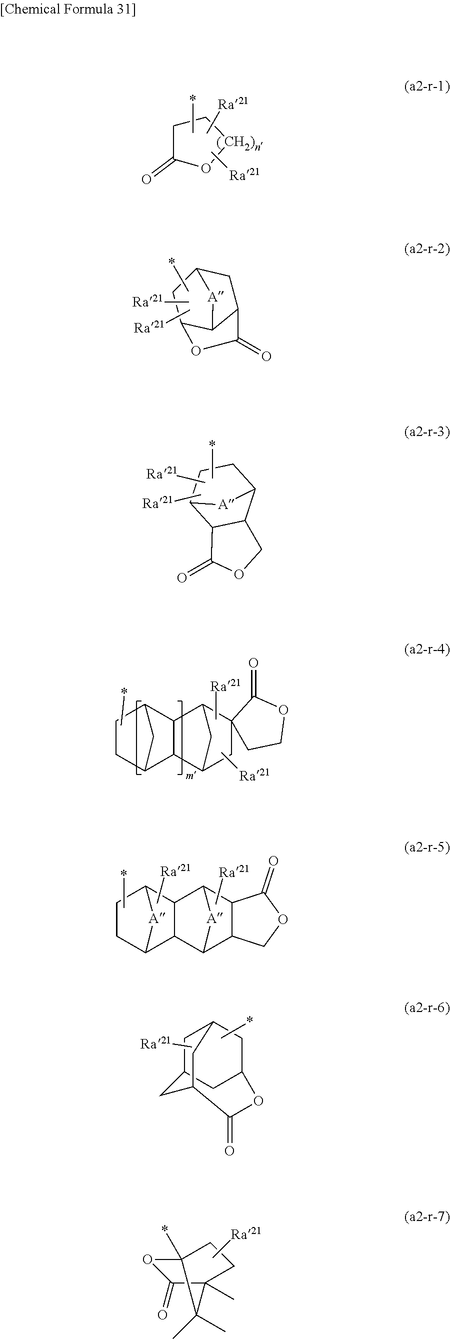

[in the formula, Ra′²¹'s are each independently a hydrogen atom, an alkyl group, an alkoxy group, a halogen atom, a halogenated alkyl group, a hydroxyl group, —COOR″, —OC(═O)R″, a hydroxyalkyl group or a cyano group; R″ is a hydrogen atom, an alkyl group or a lactone-containing cyclic group; A″ is an alkylene group having 1 to 5 carbon atoms which may contain an oxygen atom (—O—) or a sulfur atom (—S—), an oxygen atom or a sulfur atom, n′ is an integer of 0 to 2, and m′ is 0 or 1, and * indicates a bond (the same applies hereinafter)].

In General Formulae (a2-r-1) to (a2-r-7), the alkyl group for Ra′²¹ is preferably an alkyl group having 1 to 6 carbon atoms. The alkyl group is preferably linear or branched. Specific examples thereof include a methyl group, an ethyl group, a propyl group, an isopropyl group, an n-butyl group, an isobutyl group, a tert-butyl group, a pentyl group, an isopentyl group, a neopentyl group, and a hexyl group. Among these, a methyl group or an ethyl group is preferable, and a methyl group is particularly preferable.

The alkoxy group for Ra′²¹ is preferably an alkoxy group having 1 to 6 carbon atoms. The alkoxy group is preferably linear or branched. Specific examples thereof include a group in which an alkyl group exemplified as the alkyl group for Ra′²¹ is linked to an oxygen atom (—O—).

The halogen atom for Ra′²¹ is preferably a fluorine atom.

Examples of halogenated alkyl groups for Ra′²¹ include a group in which some or all of the hydrogen atoms in the alkyl group for Ra′²¹ are substituted with the halogen atoms. The halogenated alkyl group is preferably a fluorinated alkyl group and particularly preferably a perfluoroalkyl group.

In —COOR″ and —OC(═O)R″ for Ra′²¹, R″ 's are all a hydrogen atom, an alkyl group, or a lactone-containing cyclic group.

The alkyl group for R″ may be linear, branched, or cyclic, and preferably has 1 to 15 carbon atoms.

When R″ is a linear or branched alkyl group, it preferably has 1 to 10 carbon atoms, more preferably has 1 to 5 carbon atoms, and is particularly preferably a methyl group or an ethyl group.

When R″ is a cyclic alkyl group, it preferably has 3 to 15 carbon atoms, more preferably has 4 to 12 carbon atoms, and most preferably has 5 to 10 carbon atoms. Specific examples thereof include a group in which one or more hydrogen atoms have been removed from a monocycloalkane which may or may not be substituted with a fluorine atom or a fluorinated alkyl group; and a group in which one or more hydrogen atoms have been removed from a polycycloalkane such as bicycloalkane, tricycloalkane, or tetracycloalkane. More specific examples thereof include a group in which one or more hydrogen atoms have been removed from a monocycloalkane such as cyclopentane or cyclohexane; and a group in which one or more hydrogen atoms have been removed from a polycycloalkane such as adamantane, norbornane, isobornane, tricyclodecane, or tetracyclododecane.

Examples of lactone-containing cyclic groups for R″ include the same groups represented by General Formulae (a2-r-1) to (a2-r-7).

The hydroxyalkyl group for Ra′²¹ preferably has 1 to 6 carbon atoms, and specific examples thereof include a group in which at least one hydrogen atom in the alkyl group for Ra′²¹ is substituted with a hydroxyl group.

Among the above examples, Ra′²¹'s are each independently preferably a hydrogen atom or a cyano group.

In General Formulae (a2-r-2), (a2-r-3), and (a2-r-5), the alkylene group having 1 to 5 carbon atoms for A″ is preferably a linear or branched alkylene group, and examples thereof include a methylene group, an ethylene group, an n-propylene group, and an isopropylene group. When the alkylene group contains an oxygen atom or a sulfur atom, specific examples thereof include a group in which —O— or —S— is inserted into the end or between carbon atoms of the alkylene group, and for example, —O—CH₂—, —CH₂—O—CH₂—, —S—CH₂—, and —CH₂—S—CH₂— are exemplified. A″ is preferably an alkylene group having 1 to 5 carbon atoms or —O—, more preferably an alkylene group having 1 to 5 carbon atoms, and most preferably a methylene group.

Specific examples of groups represented by General Formulae (a2-r-1) to (a2-r-7) are shown below.

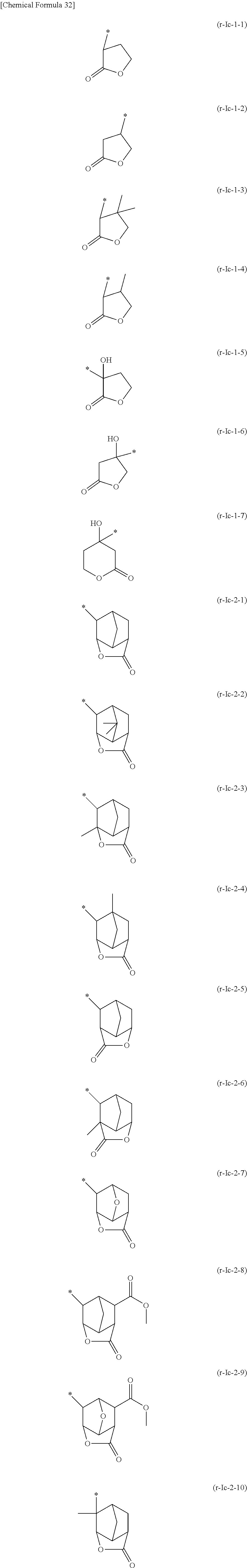

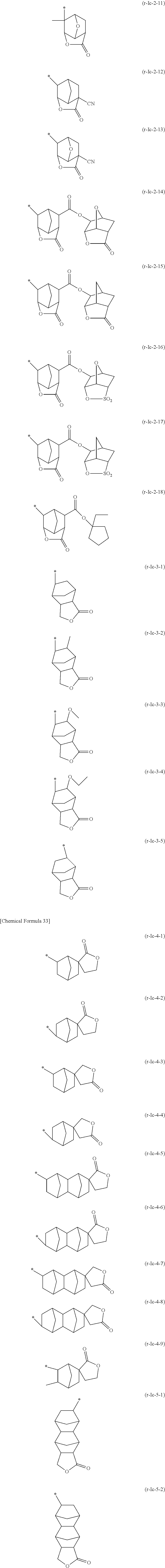

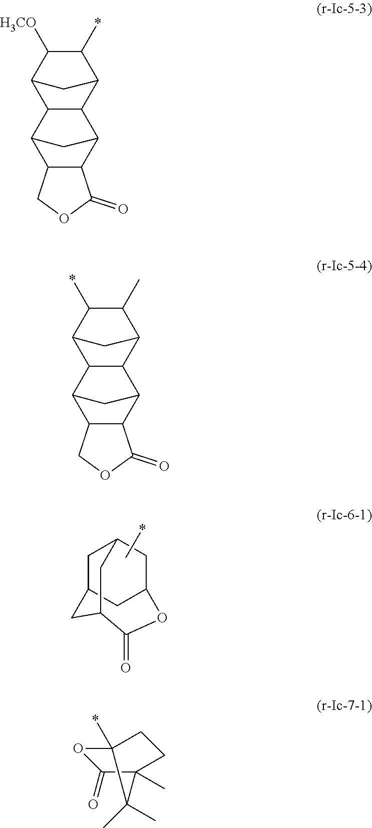

Among these, the structural unit (a2) is preferably a structural unit derived from an acrylic acid ester in which the hydrogen atom bonded to the carbon atom at the α-position may be substituted with a substituent.

Such a structural unit (a2) is preferably a structural unit represented by the following General Formula (a2-1).

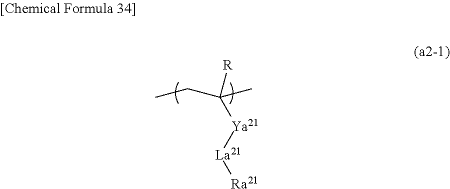

[in the formula, R is a hydrogen atom, an alkyl group having 1 to 5 carbon atoms or a halogenated alkyl group having 1 to 5 carbon atoms, Ya²¹ is a single bond or a divalent linking group, La²¹ is —O—, —COO—, —CON(R^{αx})—, —OCO—, —CONHCO— or —CONHCS—, R′ is a hydrogen atom or a methyl group, and when La²¹ is —O—, Ya²¹ does not become —CO—, and Ra²¹ is a lactone-containing cyclic group].

In Formula (a2-1), R is the same as defined above. R is preferably a hydrogen atom, an alkyl group having 1 to 5 carbon atoms or a fluorinated alkyl group having 1 to 5 carbon atoms, and in terms of industrial availability, a hydrogen atom or a methyl group is particularly preferable.

In Formula (a2-1), the divalent linking group for Ya²¹ is not particularly limited, and preferable examples thereof include a divalent hydrocarbon group which may have a substituent and a heteroatom-containing divalent linking group.

Ya²¹ is preferably a single bond, an ester bond [—C(═O)—O—], an ether bond (—O—), a linear or branched alkylene group, or a combination thereof.

In Formula (a2-1), Ya²¹ is a single bond, and La²¹ is preferably —COO— or —OCO—.

In Formula (a2-1), Ra²¹ is a lactone-containing cyclic group.

Preferable examples of lactone-containing cyclic groups for Ra²¹ include the groups represented by General Formulae (a2-r-1) to (a2-r-7).

The structural unit (a2) of the component (A1) may be of one type or of two or more types.

When the component (A1) has the structural unit (a2), the proportion of the structural unit (a2) based on a total amount (100 mol %) of all structural units constituting the component (A1) is preferably 1 to 20 mol %, more preferably 1 to 15 mol %, and still more preferably 1 to 10 mol %.

When the proportion of the structural unit (a2) is set to be equal to or larger than the preferable lower limit value, according to the above effects, the effects obtained when the structural unit (a2) is provided are sufficiently obtained, and when the proportion is set to be equal to or smaller than the upper limit value, a balance with other structural units can be achieved and various lithography properties become favorable.

### Structural Unit (a8):

The structural unit (a8) is a structural unit derived from the compound represented by the following General Formula (a8-1).

Here, those corresponding to the structural unit (a0) are excluded.

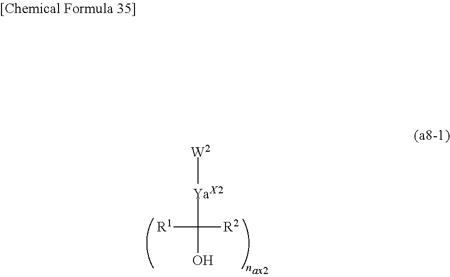

[in the formula, W² is a polymerizable group-containing group, Ya^{x2} is a single bond or a (nₐₓ₂+1)-valent linking group, Ya^{x2} and W² may form a fused ring, R¹ is a fluorinated alkyl group having 1 to 12 carbon atoms, R² is an organic group having 1 to 12 carbon atoms which may have a fluorine atom or a hydrogen atom, R² and Ya^{x2} may be bonded to each other to form a ring structure, and nₐₓ₂ is an integer of 1 to 3].

The “polymerizable group” in the polymerizable group-containing group for W² is a group that enables a compound having a polymerizable group to be polymerized by radical polymerization or the like, and is, for example, a group containing a multiple bond between carbon atoms such as an ethylenic double bond.

The polymerizable group-containing group may be a group composed of only a polymerizable group or may be a group composed of a polymerizable group and a group other than the polymerizable group. Examples of groups other than the polymerizable group include a divalent hydrocarbon group which may have a substituent and a heteroatom-containing divalent linking group.

Preferable examples of polymerizable group-containing groups include groups represented by chemical formula: C(R^{X11})(R^{X12})═C(R^{X13})-Ya^{x0}-. In the chemical formula, R^{X11}, R^{X12} and R^{X13} are each a hydrogen atom, an alkyl group having 1 to 5 carbon atoms or a halogenated alkyl group having 1 to 5 carbon atoms, and Ya^{x0} is a single bond or a divalent linking group.

Examples of fused rings formed by Ya^{x2} and W² include a fused ring formed by a polymerizable group at the W² moiety and Ya^{x2} and a fused ring formed by a group other than the polymerizable group at the W² moiety and Ya^{x2}.

The fused ring formed by Ya^{x2} and W² may have a substituent.

Specific examples of structural units (a8) are shown below.

In the following formulae, R^{α} is a hydrogen atom, a methyl group or a trifluoromethyl group.

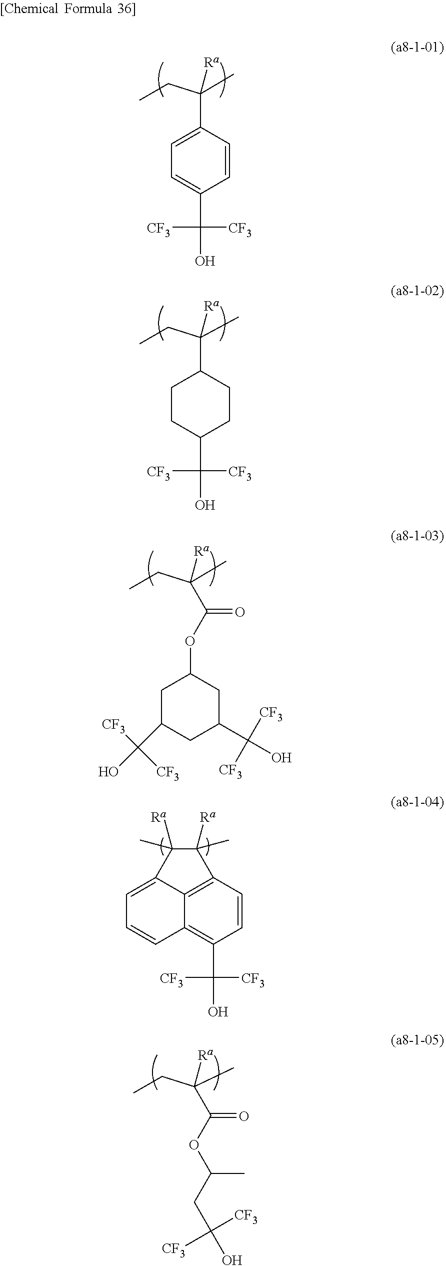

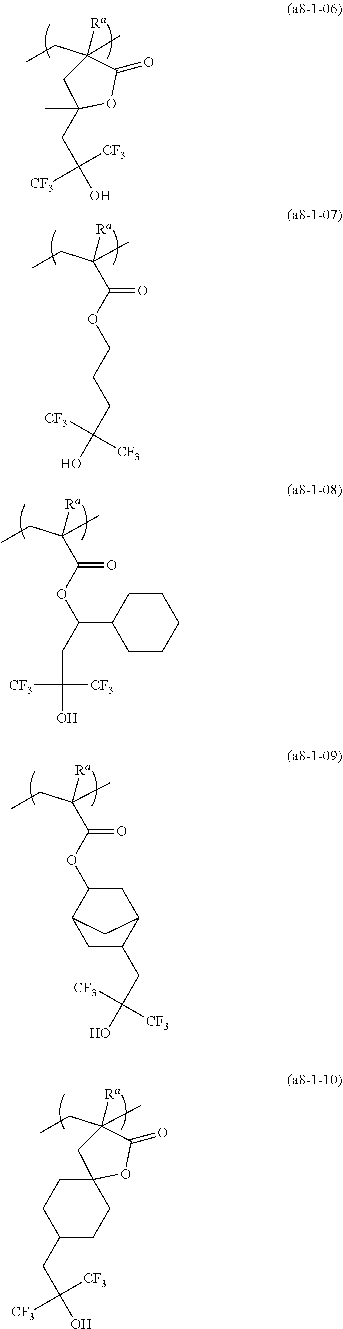

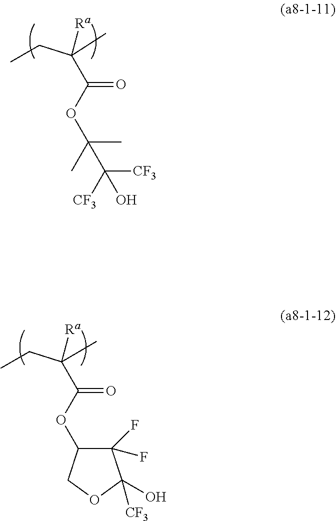

Among the above examples, the structural unit (a8) is preferably at least one selected from the group consisting of structural units represented by Chemical Formulae (a8-1-01) to (a8-1-04), (a8-1-06), (a8-1-08), (a8-1-09), and (a8-1-10), and more preferably at least one selected from the group consisting of structural units represented by Chemical Formulae (a8-1-01) to (a8-1-04), and (a8-1-09).

The structural unit (a8) of the component (A1) may be of one type or of two or more types.

The proportion of the structural unit (a8) in the component (A1) based on a total amount (100 mol %) of all structural units constituting the component (A1) is preferably 50 mol % or less and more preferably 0 to 30 mol %.

The components (A1) in the resist composition may be used alone or two or more thereof may be used in combination.

In the resist composition of the present embodiment, examples of components (A1) include high-molecular-weight compounds having a repeating structure of the structural unit (a1).

Among the above examples, preferable examples of components (A1) include high-molecular-weight compounds having a repeating structure of the structural unit (a1) and the structural unit (a10).

In a high-molecular-weight compound having a repeating structure of the structural unit (a1) and the structural unit (a10), the proportion of the structural unit (a1) based on a total amount (100 mol %) of all structural units constituting the high-molecular-weight compound is preferably 10 to 90 mol %, more preferably 20 to 80 mol %, still more preferably 30 to 70 mol %, and particularly preferably 40 to 65 mol %.

In addition, the proportion of the structural unit (a10) in the high-molecular-weight compound based on a total amount (100 mol %) of all structural units constituting the high-molecular-weight compound is preferably 10 to 90 mol %, more preferably 20 to 80 mol %, still more preferably 30 to 70 mol %, and particularly preferably 40 to 60 mol %.

The component (A1) can be produced by dissolving monomers from which structural units are derived in a polymerization solvent, and adding a radical polymerization initiator such as azobisisobutyronitrile (AIBN), or dimethyl azobisisobutyrate (for example, V-601) thereto to cause polymerization.

Alternatively, the component (A1) can be produced by dissolving a monomer from which the structural unit (a1) is derived, and as necessary, a monomer from which a structural unit (for example, structural unit (a10)) other than the structural unit (a1) is derived in a polymerization solvent, adding the above radical polymerization initiator thereto to cause polymerization, and then performing a deprotection reaction.

Here, during polymerization, for example, when a chain transfer agent such as HS—CH₂—CH₂—CH₂—C(CF₃)₂—OH is used in combination, a —C(CF₃)₂—OH group may be introduced into the end. Accordingly, a copolymer in which a hydroxyalkyl group in which some hydrogen atoms in the alkyl group are substituted with fluorine atoms is introduced is effective in reducing development defects and reducing LER (line edge roughness: non-uniform unevenness of line sidewalls).

The weight average molecular weight (Mw) (in terms of polystyrene determined through gel permeation chromatography (GPC)) of the component (A1) is not particularly limited, and is preferably 1,000 to 50,000, more preferably 2,000 to 30,000, and still more preferably 3,000 to 20,000.

When the Mw of the component (A1) is equal to or smaller than the preferable upper limit value in this range, the component has sufficient solubility in a resist solvent for use as a resist, and when the Mw of the component (A1) is equal to or larger than the preferable lower limit value in this range, the component has favorable dry etching resistance and a favorable cross-sectional shape of the resist pattern.

The dispersity (Mw/Mn) of the component (A1) is not particularly limited, and is preferably 1.0 to 4.0, more preferably 1.0 to 3.0, and particularly preferably 1.0 to 2.0. Here, Mn is the number average molecular weight.

### Component (A2)

In the resist composition of the present embodiment, as the component (A), a base component (hereinafter referred to as a “component (A2)”) that does not correspond to the component (A1) and its solubility in a development solution changes under the action of an acid may be used in combination.

The component (A2) is not particularly limited, and any component selected from among many components conventionally known as base components for chemically amplified resist compositions may be used.

As the component (A2), a high-molecular-weight compound or a low-molecular-weight compound may be used alone or two or more thereof may be used in combination.

The proportion of the component (A1) in the component (A) based on a total mass of the component (A) is preferably 25 mass % or more, more preferably 50 mass % or more, still more preferably 75 mass % or more, and may be 100 mass %. When the proportion is 25 mass % or more, it is easier to form a resist pattern having various excellent lithography properties such as high sensitivity and resolution, and improved roughness.

In the resist composition of the present embodiment, the content of the component (A) may be adjusted depending on the thickness of the resist film to be formed and the like.

### <<Acid Generator Component (B)>>

The resist composition of the present embodiment preferably further contains an acid generator component (B) that generates an acid upon exposure.

The component (B) is not particularly limited, and any of components that have been previously proposed as acid generators for chemically amplified resist compositions can be used.

Examples of such acid generators include onium salt-based acid generators such as iodonium salts and sulfonium salts, and oxime sulfonate-based acid generators; diazomethane-based acid generators such as bisalkyl or bisarylsulfonyldiazomethanes, and poly(bissulfonyl)diazomethanes; and other types such as nitrobenzyl sulfonate-based acid generators, iminosulfonate-based acid generators, and disulfone-based acid generators.

Examples of onium salt-based acid generators include a compound represented by the following General Formula (b-1) (hereinafter referred to as a “component (b-1)”), a compound represented by General Formula (b-2) (hereinafter referred to as a “component (b-2)”) and a compound represented by General Formula (b-3) (hereinafter referred to as a “component (b-3)”).

Examples of onium salt-based acid generators include a compound represented by the following General Formula (b-1) (hereinafter referred to as a “component (b-1)”), a compound represented by General Formula (b-2) (hereinafter referred to as a “component (b-2)”) and a compound represented by General Formula (b-3) (hereinafter referred to as a “component (b-3)”).

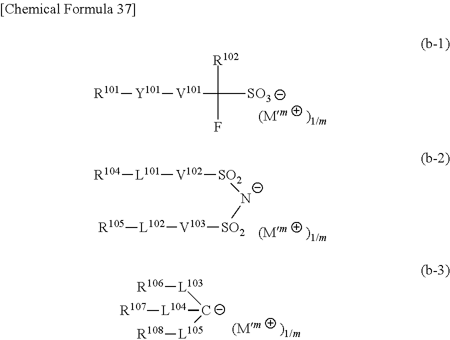

[in the formula, R¹⁰¹ and R¹⁰⁴ to R¹⁰⁸ are each independently a cyclic group which may have a substituent, a chain alkyl group which may have a substituent, or a chain alkenyl group which may have a substituent, R¹⁰⁴ and R¹⁰⁵ may be bonded to each other to form a ring structure, R¹⁰² is a fluorinated alkyl group having 1 to 5 carbon atoms or a fluorine atom, Y¹⁰¹ is an oxygen atom-containing divalent linking group or a single bond, V¹⁰¹ to V¹⁰³ are each independently a single bond, an alkylene group or a fluorinated alkylene group, L¹⁰¹ to L¹⁰² are each independently a single bond or an oxygen atom, L¹⁰³ to L¹⁰⁵ are each independently a single bond, —CO— or —SO₂—, m is an integer of 1 or more, and M′^{m+} is an m-valent onium cation].

### {Anion Moiety}

Anion in Component (b-1)

In Formula (b-1), R¹⁰¹ is a cyclic group which may have a substituent, a chain alkyl group which may have a substituent, or a chain alkenyl group which may have a substituent.

Cyclic Group which May have a Substituent:

The cyclic group is preferably a cyclic hydrocarbon group, and the cyclic hydrocarbon group may be an aromatic hydrocarbon group or an aliphatic hydrocarbon group. The aliphatic hydrocarbon group is a hydrocarbon group having no aromaticity. In addition, the aliphatic hydrocarbon group is preferably saturated.

The aromatic hydrocarbon group for R¹⁰¹ is a hydrocarbon group having an aromatic ring. The aromatic hydrocarbon group preferably has 3 to 30 carbon atoms, more preferably has 5 to 30 carbon atoms, still more preferably has 5 to 20 carbon atoms, particularly preferably has 6 to 15 carbon atoms, and most preferably has 6 to 10 carbon atoms. Here, the number of carbon atoms does not include the number of carbon atoms in the substituent.

Specific examples of aromatic rings in the aromatic hydrocarbon group for R¹⁰¹ include benzene, fluorene, naphthalene, anthracene, phenanthrene, biphenyl and aromatic heterocycles in which some carbon atoms constituting these aromatic rings are substituted with heteroatoms. Examples of heteroatoms in aromatic heterocycles include an oxygen atom, a sulfur atom, and a nitrogen atom.

Specific examples of aromatic hydrocarbon groups for R¹⁰¹ include a group in which one hydrogen atom has been removed from the aromatic ring (an aryl group: for example, a phenyl group and a naphthyl group, etc.), and a group in which one hydrogen atom in the aromatic ring is substituted with an alkylene group (for example, a benzyl group, a phenethyl group, a 1-naphthyl methyl group, etc.). The alkylene group (the alkyl chain in the arylalkyl group) preferably has 1 to 4 carbon atoms, more preferably has 1 to 2 carbon atoms, and particularly preferably has 1 carbon atom.

Examples of cyclic aliphatic hydrocarbon groups for R¹⁰¹ include an aliphatic hydrocarbon group containing a ring in the structure.

Examples of aliphatic hydrocarbon groups containing a ring in the structure include an alicyclic hydrocarbon group (a group in which one hydrogen atom has been removed from an aliphatic hydrocarbon ring), a group in which an alicyclic hydrocarbon group is bonded to the end of a linear or branched aliphatic hydrocarbon group, and a group in which an alicyclic hydrocarbon group is inserted midway along a linear or branched aliphatic hydrocarbon group.

The alicyclic hydrocarbon group preferably has 3 to 20 carbon atoms and more preferably has 3 to 12 carbon atoms.

The alicyclic hydrocarbon group may be a polycyclic group or a monocyclic group. The monocyclic alicyclic hydrocarbon group is preferably a group in which one or more hydrogen atoms have been removed from a monocycloalkane. The monocycloalkane preferably has 3 to 6 carbon atoms, and specific examples thereof include cyclopentane and cyclohexane. The polycyclic alicyclic hydrocarbon group is preferably a group in which one or more hydrogen atoms have been removed from a polycycloalkane, and the polycycloalkane preferably has 7 to 30 carbon atoms. Among these, the polycycloalkane is more preferably a polycycloalkane having a crosslinked ring type polycyclic framework such as adamantane, norbornane, isobornane, tricyclodecane, and tetracyclododecane; or a polycycloalkane having a fused ring type polycyclic framework such as a cyclic group having a steroid framework.

Among these, the cyclic aliphatic hydrocarbon group for R¹⁰¹ is preferably a group in which one or more hydrogen atoms have been removed from a monocycloalkane or polycycloalkane, more preferably a group in which one hydrogen atom has been removed from a polycycloalkane, still more preferably an adamantyl group or a norbornyl group, and particularly preferably an adamantyl group.

The linear aliphatic hydrocarbon group which may be bonded to an alicyclic hydrocarbon group preferably has 1 to 10 carbon atoms, more preferably has 1 to 6 carbon atoms, still more preferably has 1 to 4 carbon atoms, and most preferably has 1 to 3 carbon atoms. The linear aliphatic hydrocarbon group is preferably a linear alkylene group, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group which may be bonded to an alicyclic hydrocarbon group preferably has 2 to 10 carbon atoms, more preferably has 3 to 6 carbon atoms, still more preferably has 3 or 4 carbon atoms, and most preferably has 3 carbon atoms. The branched aliphatic hydrocarbon group is preferably a branched alkylene group, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. The alkyl group in the alkylalkylene group is preferably a linear alkyl group having 1 to 5 carbon atoms.

In addition, the cyclic hydrocarbon group for R¹⁰¹ may contain a heteroatom such as a heterocycle. Specific examples thereof include lactone-containing cyclic groups represented by General Formulae (a2-r-1) to (a2-r-7), —SO₂-containing cyclic groups represented by the following General Formulae (b5-r-1) to (b5-r-4), and other heterocyclic groups represented by the following Chemical Formulae (r-hr-1) to (r-hr-16). In the formulae, * indicates a bond bonded to Y^{10′} in Formula (b-1).

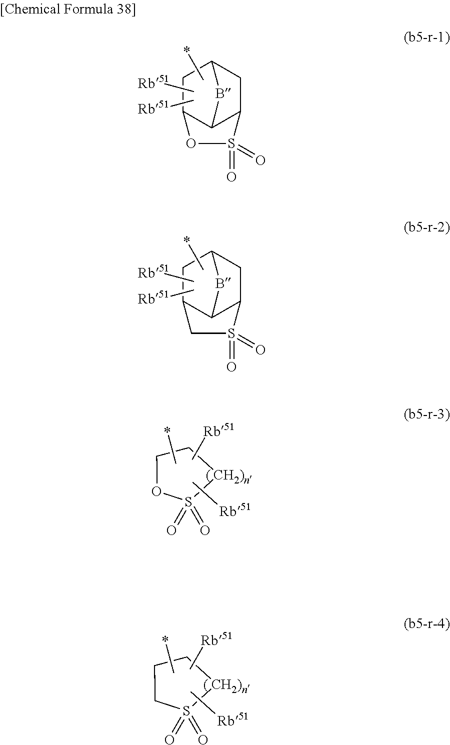

[in the formula, Rb′⁵¹'s are each independently a hydrogen atom, an alkyl group, an alkoxy group, a halogen atom, a halogenated alkyl group, a hydroxyl group, —COOR″, —OC(═O)R″, a hydroxyalkyl group or a cyano group; R″ is a hydrogen atom, an alkyl group, a lactone-containing cyclic group, or a —SO₂-containing cyclic group; B″ is an alkylene group having 1 to 5 carbon atoms which may contain an oxygen atom or a sulfur atom, an oxygen atom or a sulfur atom, n′ is an integer of 0 to 2, and * indicates a bond].

In General Formulae (b5-r-1) to (b5-r-2), B″ is an alkylene group having 1 to 5 carbon atoms which may contain an oxygen atom or a sulfur atom, an oxygen atom or a sulfur atom.

B″ is preferably an alkylene group having 1 to 5 carbon atoms or —O—, more preferably an alkylene group having 1 to 5 carbon atoms, and still more preferably a methylene group.

In General Formulae (b5-r-1) to (b5-r-4), Rb′⁵¹'s are each independently a hydrogen atom, an alkyl group, an alkoxy group, a halogen atom, a halogenated alkyl group, a hydroxyl group, —COOR″, —OC(═O)R″, a hydroxyalkyl group or a cyano group, and among these, they are each independently preferably a hydrogen atom or a cyano group.

Specific examples of groups represented by General Formulae (b5-r-1) to (b5-r-4) are shown below. In the formulae, “Ac” is an acetyl group.

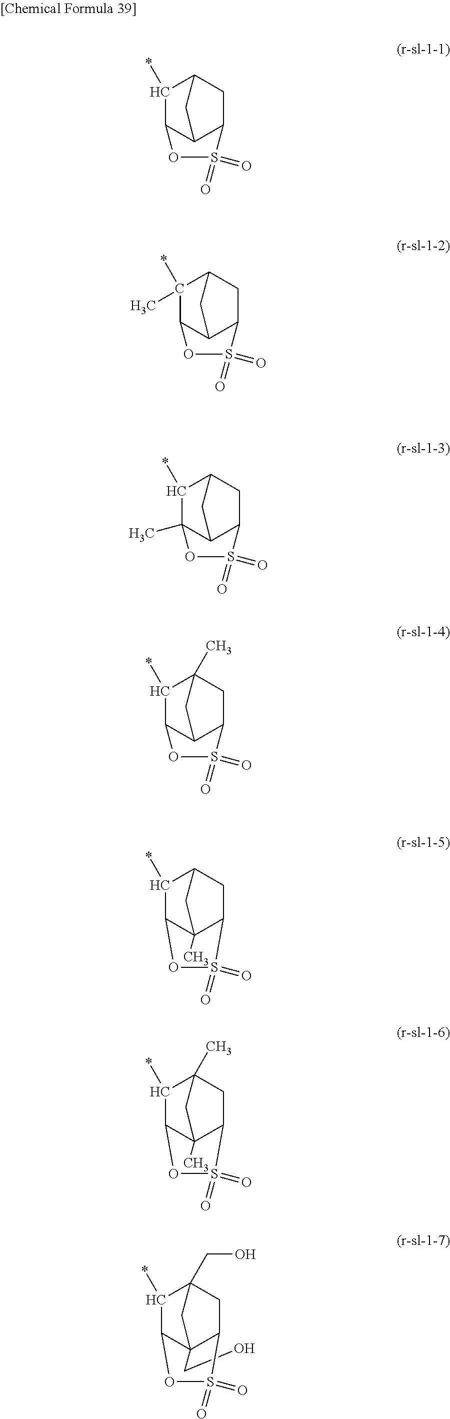

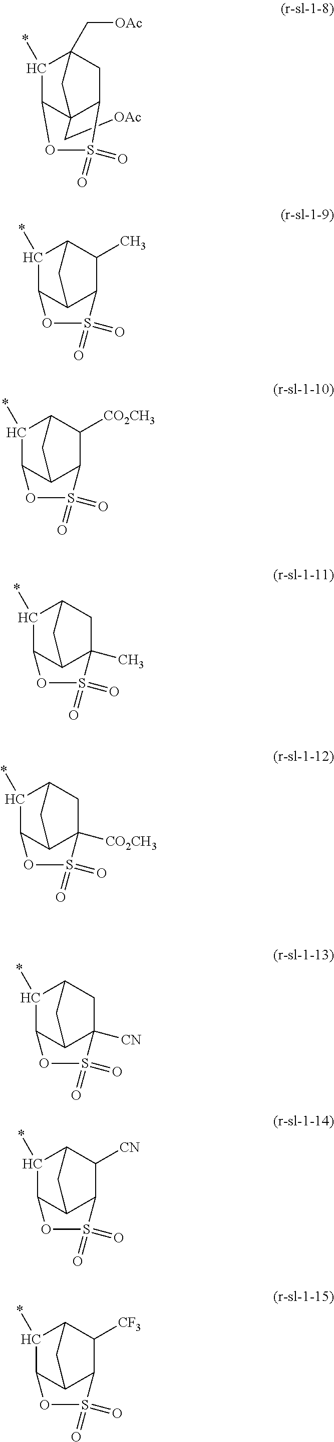

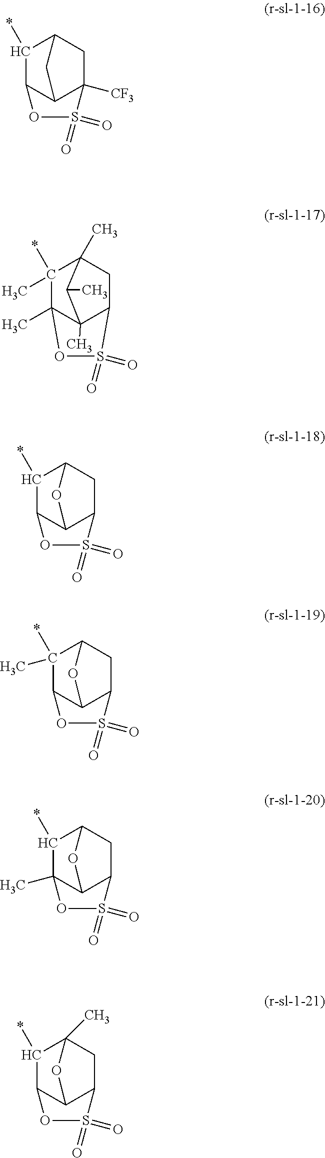

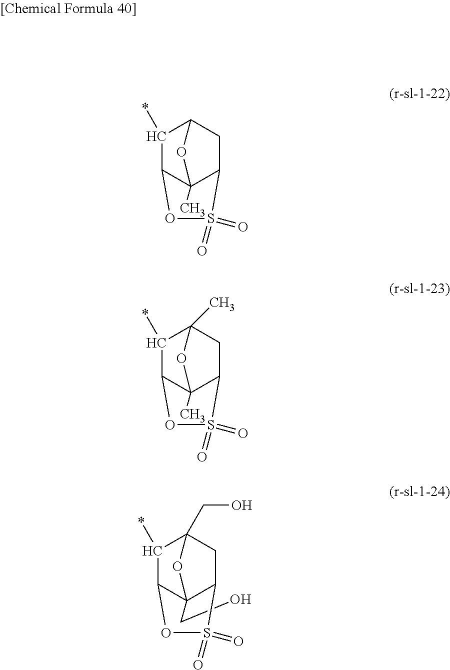

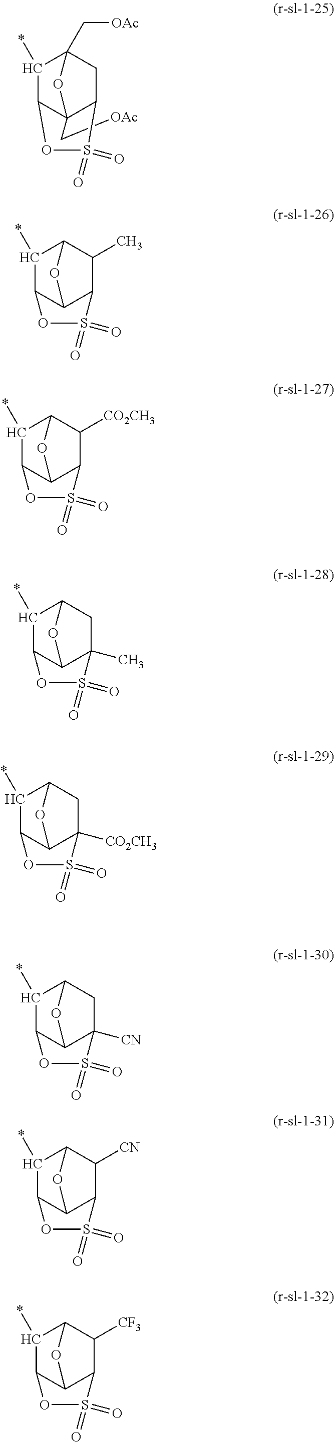

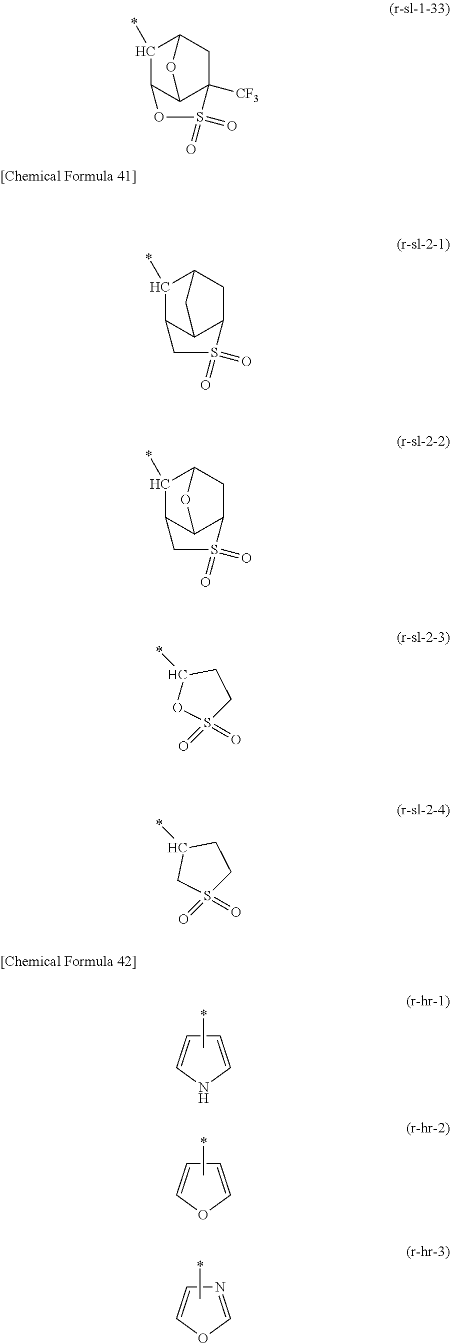

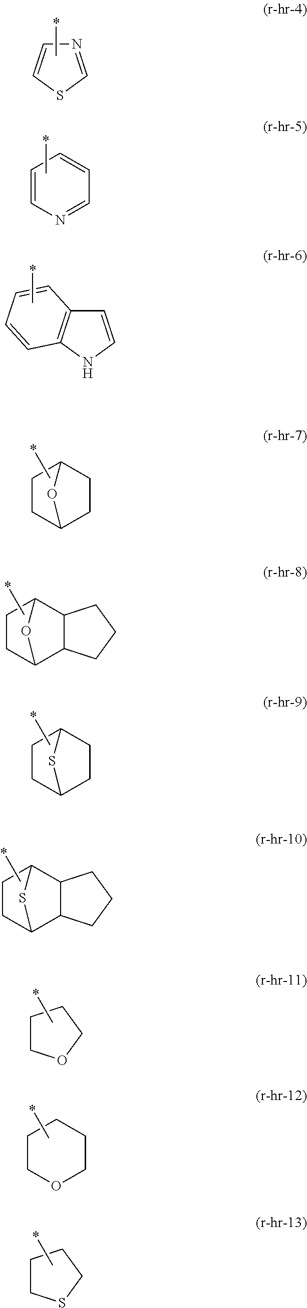

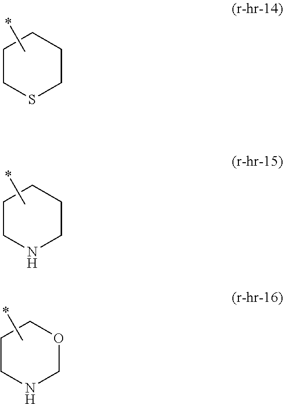

Examples of substituents in the cyclic group for R¹⁰¹ include an alkyl group, an alkoxy group, a halogen atom, a halogenated alkyl group, a hydroxyl group, a carbonyl group, and a nitro group.

The alkyl group as a substituent is preferably an alkyl group having 1 to 5 carbon atoms.

The alkoxy group as a substituent is preferably an alkoxy group having 1 to 5 carbon atoms, more preferably a methoxy group, an ethoxy group, an n-propoxy group, an iso-propoxy group, an n-butoxy group, or a tert-butoxy group, and most preferably a methoxy group or an ethoxy group.

The halogen atom as a substituent is preferably a fluorine atom, a bromine atom, or an iodine atom.

Examples of halogenated alkyl groups as a substituent include an alkyl group having 1 to 5 carbon atoms, for example, a group in which some or all of the hydrogen atoms are substituted with the halogen atoms such as a methyl group, an ethyl group, a propyl group, an n-butyl group, and a tert-butyl group.

The carbonyl group as a substituent is a group that substitutes a methylene group (—CH₂—) constituting a cyclic hydrocarbon group.

The cyclic hydrocarbon group for R¹⁰¹ may be a fused cyclic group containing a fused ring in which an aliphatic hydrocarbon ring and an aromatic ring are fused. Examples of fused rings include a ring in which one or more aromatic rings are fused to a polycycloalkane having a crosslinked ring type polycyclic framework. Specific examples of crosslinked ring type polycycloalkanes include bicycloalkanes such as bicyclo[2.2.1]heptane(norbornane) and bicyclo[2.2.2]octane. The fused ring type is preferably a group containing a fused ring in which 2 or 3 aromatic rings are fused to a bicycloalkane, and more preferably a group containing a fused ring in which 2 or 3 aromatic rings are fused to bicyclo[2.2.2]octane. Specific examples of fused cyclic groups for R¹⁰¹ include those represented by the following Formulae (r-br-1) to (r-br-2). In the formulae, * indicates a bond bonded to Y¹⁰¹ in Formula (b-1).

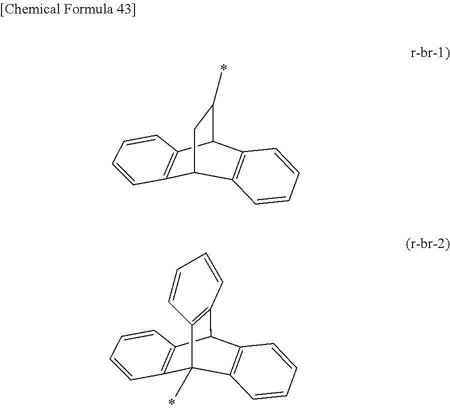

Examples of substituents that the fused cyclic group for R¹⁰¹ may have include an alkyl group, an alkoxy group, a halogen atom, a halogenated alkyl group, a hydroxyl group, a carbonyl group, a nitro group, an aromatic hydrocarbon group, and an alicyclic hydrocarbon group.

Examples of alkyl groups, alkoxy groups, halogen atoms, and halogenated alkyl groups as substituents for the fused cyclic groups include the same as those exemplified as the substituents for the cyclic groups for R¹⁰ 1.

Examples of aromatic hydrocarbon groups as a substituent for the fused cyclic group include a group in which one hydrogen atom has been removed from an aromatic ring (an aryl group: for example, a phenyl group, a naphthyl group, etc.), a group in which one hydrogen atom in the aromatic ring is substituted with an alkylene group (for example, arylalkyl groups such as a benzyl group, a phenethyl group, a 1-naphthyl methyl group, a 2-naphthyl methyl group, a 1-naphthyl ethyl group, and a 2-naphthyl ethyl group), and heterocyclic groups represented by Formulae (r-hr-1) to (r-hr-6).

Examples of alicyclic hydrocarbon groups as a substituent for the fused cyclic group include a group in which one hydrogen atom has been removed from a monocycloalkane such as cyclopentane and cyclohexane; a group in which one hydrogen atom has been removed from a polycycloalkane such as adamantane, norbornane, isobornane, tricyclodecane, and tetracyclododecane; lactone-containing cyclic groups represented by General Formulae (a2-r-1) to (a2-r-7); —SO₂-containing cyclic groups represented by General Formulae (b5-r-1) to (b5-r-4); and heterocyclic groups represented by Formulae (r-hr-7) to (r-hr-16).

Chain Alkyl Group which May have a Substituent:

The chain alkyl group for R¹⁰¹ may be either linear or branched.

The linear alkyl group preferably has 1 to 20 carbon atoms, more preferably has 1 to 15 carbon atoms, and most preferably has 1 to 10 carbon atoms.

The branched alkyl group preferably has 3 to 20 carbon atoms, more preferably has 3 to 15 carbon atoms, and most preferably has 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain Alkenyl Group which May have a Substituent:

The chain alkenyl group for R¹⁰¹ may be either linear or branched, and preferably has 2 to 10 carbon atoms, more preferably has 2 to 5 carbon atoms, still more preferably has 2 to 4 carbon atoms, and particularly preferably has 3 carbon atoms. Examples of linear alkenyl groups include a vinyl group, a propenyl group (allyl group), and a butynyl group. Examples of branched alkenyl groups include a 1-methylvinyl group, a 2-methylvinyl group, a 1-methylpropenyl group, and a 2-methylpropenyl group.

Among the above examples, the chain alkenyl group is preferably a linear alkenyl group, more preferably a vinyl group or a propenyl group, and particularly preferably a vinyl group.

Examples of substituents in the chain alkyl group or alkenyl group for R¹⁰¹ include an alkoxy group, a halogen atom, a halogenated alkyl group, a hydroxyl group, a carbonyl group, a nitro group, an amino group, and the cyclic groups for R¹⁰ 1.

In Formula (b-1), Y¹⁰¹ is a single bond or an oxygen atom-containing divalent linking group.

When Y¹⁰¹ is an oxygen atom-containing divalent linking group, Y¹⁰¹ may contain an atom other than an oxygen atom. Examples of atoms other than an oxygen atom include a carbon atom, a hydrogen atom, a sulfur atom, and a nitrogen atom.

Examples of oxygen atom-containing divalent linking groups include linking groups represented by the following General Formulae (y-a1-1) to (y-a1-7). Here, in the following General Formulae (y-a1-1) to (y-a1-7), one bonded to R¹⁰¹ in Formula (b-1) is V′¹⁰¹ in the following General Formulae (y-a1-1) to (y-a1-7).

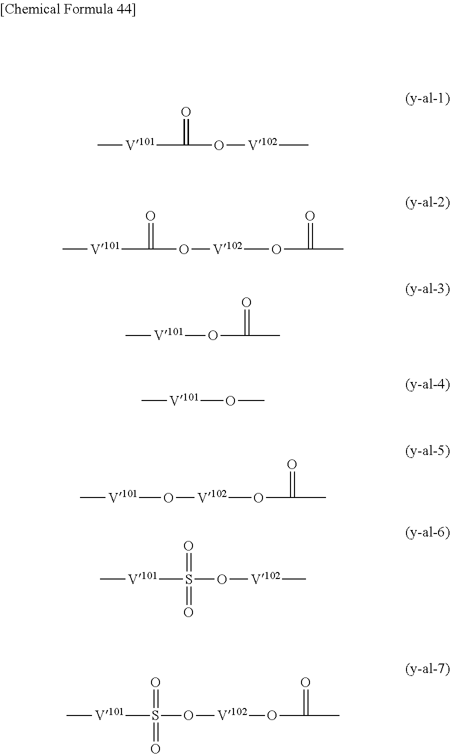

[in the formula, V′¹⁰¹ is a single bond or an alkylene group having 1 to 5 carbon atoms, and V′¹⁰² is a divalent saturated hydrocarbon group having 1 to 30 carbon atoms].

The divalent saturated hydrocarbon group for V′¹⁰² is preferably an alkylene group having 1 to 30 carbon atoms, more preferably an alkylene group having 1 to 10 carbon atoms, and still more preferably an alkylene group having 1 to 5 carbon atoms.

The alkylene group for V′¹⁰¹ and V′¹⁰² may be a linear alkylene group or a branched alkylene group, and is preferably a linear alkylene group.

Specific examples of alkylene groups for V′¹⁰¹ and V′¹⁰² include a methylene group [—CH₂—]; alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; an ethylene group [—CH₂CH₂—]; alkylethylene groups such as —CH(CH₃)CH₂—, —CH(CH₃)CH(CH₃)—, —C(CH₃)₂CH₂—, and —CH(CH₂CH₃)CH₂—; a trimethylene group (n-propylene group) [—CH₂CH₂CH₂—]; alkyltrimethylene groups such as —CH(CH₃)CH₂CH₂— and —CH₂CH(CH₃)CH₂—; a tetramethylene group [—CH₂CH₂CH₂CH₂—]; alkyltetramethylene groups such as —CH(CH₃)CH₂CH₂CH₂— and —CH₂CH(CH₃)CH₂CH₂—; and a pentamethylene group [—CH₂CH₂CH₂CH₂CH₂—].

In addition, some methylene groups in the alkylene group for V′¹⁰¹ or V′¹⁰² may be substituted with a divalent aliphatic cyclic group having 5 to 10 carbon atoms. The aliphatic cyclic group is preferably a divalent group in which a hydrogen atom has been additionally removed from a cyclic aliphatic hydrocarbon group (a monocyclic aliphatic hydrocarbon group, and a polycyclic aliphatic hydrocarbon group) for Ra′³ in Formula (a1-r-1), and more preferably a cyclohexylene group, a 1,5-adamantyl group or a 2,6-adamantyl group.

In Formula (b-1), V¹⁰¹ is a single bond, an alkylene group or a fluorinated alkylene group. Among these, V¹⁰¹ is preferably a single bond or a linear fluorinated alkylene group having 1 to 4 carbon atoms.

In Formula (b-1), R¹⁰² is a fluorine atom or a fluorinated alkyl group having 1 to 5 carbon atoms. R¹⁰² is preferably a fluorine atom or a perfluoroalkyl group having 1 to 5 carbon atoms, and more preferably a fluorine atom.

Specific examples of anion moieties represented by Formula (b-1) include fluorinated alkylsulfonate anions such as a trifluoromethanesulfonate anion and a perfluorobutanesulfonate anion for example, when Y¹⁰¹ is a single bond; and anions represented by the following Formulae (an-1) to (an-3) when Y¹⁰¹ is an oxygen atom-containing divalent linking group.

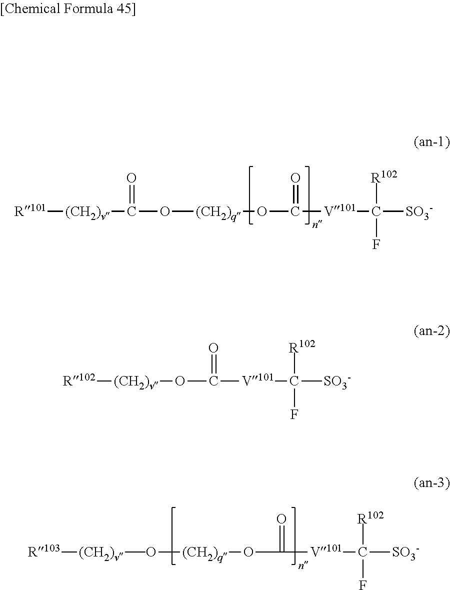

[in the formula, R″¹⁰¹ is an aliphatic cyclic group which may have a substituent, a monovalent heterocyclic group represented by each of Chemical Formulae (r-hr-1) to (r-hr-6), a fused cyclic group represented by Formula (r-br-1) or (r-br-2), a chain alkyl group which may have a substituent or an aromatic cyclic group which may have a substituent, R″¹⁰² is an aliphatic cyclic group which may have a substituent, a fused cyclic group represented by Formula (r-br-1) or (r-br-2), a lactone-containing cyclic group represented by each of General Formulae (a2-r-1), and (a2-r-3) to (a2-r-7), or a —SO₂-containing cyclic group represented by each of General Formulae (b5-r-1) to (b5-r-4), R″¹⁰³ is an aromatic cyclic group which may have a substituent, an aliphatic cyclic group which may have a substituent, or a chain alkenyl group which may have a substituent, V″¹⁰¹ is a single bond, an alkylene group having 1 to 4 carbon atoms, or a fluorinated alkylene group having 1 to 4 carbon atoms, R¹⁰² is a fluorine atom or a fluorinated alkyl group having 1 to 5 carbon atoms, v″ 's are each independently an integer of 0 to 3, q″ 's are each independently an integer of 0 to 20, and n″ is 0 or 1].

The aliphatic cyclic group which may have a substituent for R″¹⁰¹, R″¹⁰² and R″¹⁰³ is preferably a group exemplified as the cyclic aliphatic hydrocarbon group for R¹⁰¹ in Formula (b-1). Examples of substituents include the same substituents that may substitute the cyclic aliphatic hydrocarbon group for R¹⁰¹ in Formula (b-1).

The aromatic cyclic group which may have a substituent for R″¹⁰¹ and R″¹⁰³ is preferably a group exemplified as the aromatic hydrocarbon group in the cyclic hydrocarbon group for R¹⁰¹ in Formula (b-1). Examples of substituents include the same substituents that may substitute the aromatic hydrocarbon group for R¹⁰¹ in Formula (b-1).

The chain alkyl group which may have a substituent for R″¹⁰¹ is preferably a group exemplified as the chain alkyl group for R¹⁰¹ in Formula (b-1).

The chain alkenyl group which may have a substituent for R″¹⁰³ is preferably a group exemplified as the chain alkenyl group for R¹⁰¹ in Formula (b-1).

Anion in Component (b-2)

In Formula (b-2), R¹⁰⁴ and R¹⁰⁵ are each independently a cyclic group which may have a substituent, a chain alkyl group which may have a substituent, or a chain alkenyl group which may have a substituent, and examples thereof include the same groups as for R¹⁰¹ in Formula (b-1). Here, R¹⁰⁴ and R¹⁰⁵ may be bonded to each other to form a ring.

R¹⁰⁴ and R¹⁰⁵ are preferably a chain alkyl group which may have a substituent and more preferably a linear or branched alkyl group or a linear or branched fluorinated alkyl group.

The chain alkyl group preferably has 1 to 10 carbon atoms, more preferably has 1 to 7 carbon atoms, and still more preferably has 1 to 3 carbon atoms. The number of carbon atoms in the chain alkyl group for R¹⁰⁴ and R¹⁰⁵ is preferably a smaller number within the above range of the number of carbon atoms, for reasons such as favorable solubility in a resist solvent. In addition, in the chain alkyl group for R¹⁰⁴ and R¹⁰⁵, a larger number of hydrogen atoms that are substituted with fluorine atoms is preferable because the strength of the acid is stronger and the transparency to high-energy light of 250 nm or less and electron beams is improved. The proportion of fluorine atoms in the chain alkyl group, that is, the fluorination rate, is preferably 70 to 100%, and more preferably 90 to 100%, and a perfluoroalkyl group in which all hydrogen atoms are substituted with fluorine atoms is most preferable.

In Formula (b-2), V¹⁰² and V¹⁰³ are each independently a single bond, an alkylene group, or a fluorinated alkylene group, and examples thereof include the same groups as for V¹⁰¹ in Formula (b-1).

In Formula (b-2), L¹⁰¹ and L¹⁰² are each independently a single bond or an oxygen atom.

Anion in Component (b-3)

In Formula (b-3), R¹⁰⁶ to R¹⁰⁸ are each independently a cyclic group which may have a substituent, a chain alkyl group which may have a substituent, or a chain alkenyl group which may have a substituent, and examples thereof include the same groups as for R¹⁰¹ in Formula (b-1).

In Formula (b-3), L¹⁰³ to L¹⁰⁵ are each independently a single bond, —CO— or —SO₂—.

Among the above examples, the anion moiety of the component (B) is preferably an anion in the component (b-1).

### {Cation Moiety}

In Formula (b-1), Formula (b-2), and Formula (b-3), M′^{m+} is an m-valent onium cation. Among these, a sulfonium cation and an iodonium cation are preferable.
m is an integer of 1 or more.

Examples of preferable cation moieties ((M′^{m+})_{1/m}) include organic cations represented by the following General Formulae (ca-1) to (ca-3).

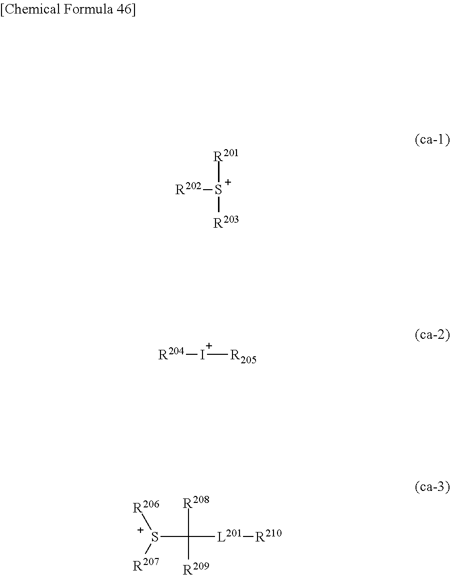

[in the formula, R²⁰¹ to R²⁰⁷ are each independently an aryl group, alkyl group or alkenyl group which may have a substituent, R²⁰¹ to R²⁰³ and R²⁰⁶ to R²⁰⁷ may be bonded to each other to form a ring together with a sulfur atom in the formula, R²⁰⁸ and R²⁰⁹ are each independently a hydrogen atom or an alkyl group having 1 to 5 carbon atoms, R²¹⁰ is an aryl group which may have a substituent, an alkyl group which may have a substituent, an alkenyl group which may have a substituent, or a —SO₂-containing cyclic group which may have a substituent, and L²⁰¹ is —C(═O)— or —C(═O)—O—].

In General Formulae (ca-1) to (ca-3), examples of aryl groups for R²⁰¹ to R²⁰⁷ include an unsubstituted aryl group having 6 to 20 carbon atoms, and a phenyl group and a naphthyl group are preferable.

The alkyl group for R²⁰¹ to R²⁰⁷ is a chain or cyclic alkyl group and preferably has 1 to 30 carbon atoms.

The alkenyl group for R²⁰¹ to R²⁰⁷ preferably has 2 to 10 carbon atoms.

Examples of substituents that R²⁰¹ to R²⁰⁷, and R²¹⁰ may have include an alkyl group, a halogen atom, a halogenated alkyl group, a carbonyl group, a cyano group, an amino group, an aryl group, and groups represented by the following General Formulae (ca-r-1) to (ca-r-7).

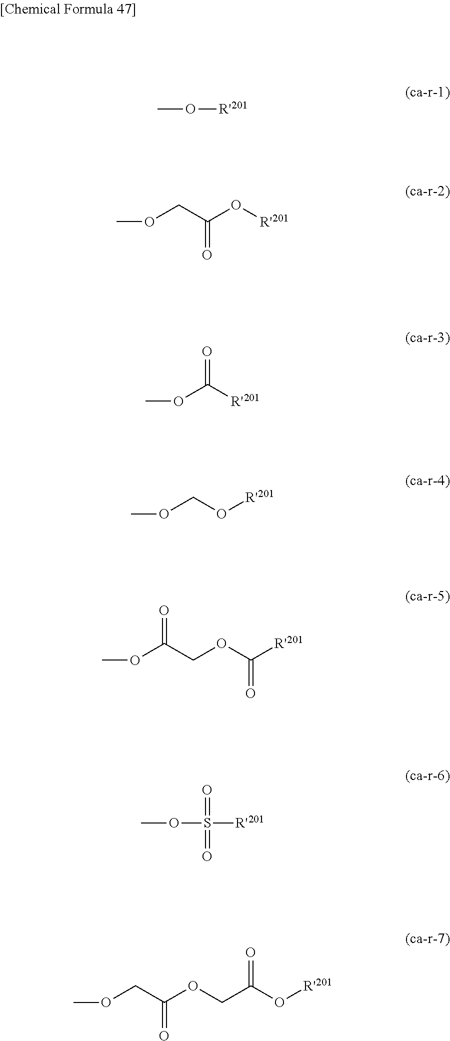

[in the formula, R′²⁰¹'s are each independently a hydrogen atom, a cyclic group which may have a substituent, chain alkyl group which may have a substituent, or a chain alkenyl group which may have a substituent].
Cyclic Group which May have a Substituent:

The cyclic group is preferably a cyclic hydrocarbon group, and the cyclic hydrocarbon group may be an aromatic hydrocarbon group or an aliphatic hydrocarbon group. The aliphatic hydrocarbon group is a hydrocarbon group having no aromaticity. In addition, the aliphatic hydrocarbon group may be saturated or unsaturated, and is generally preferably saturated.

The aromatic hydrocarbon group for R′²⁰¹ is a hydrocarbon group having an aromatic ring. The aromatic hydrocarbon group preferably has 3 to 30 carbon atoms, more preferably has 5 to 30 carbon atoms, still more preferably has 5 to 20 carbon atoms, particularly preferably has 6 to 15 carbon atoms, and most preferably has 6 to 10 carbon atoms. Here, the number of carbon atoms does not include the number of carbon atoms in the substituent.

Specific examples of aromatic rings in the aromatic hydrocarbon group for R′²⁰¹ include benzene, fluorene, naphthalene, anthracene, phenanthrene, biphenyl and aromatic heterocycles in which some carbon atoms constituting these aromatic rings are substituted with heteroatoms. Examples of heteroatoms in aromatic heterocycles include an oxygen atom, a sulfur atom, and a nitrogen atom.

Specific examples of aromatic hydrocarbon groups for R′²⁰¹ include a group in which one hydrogen atom has been removed from the aromatic ring (an aryl group: for example, a phenyl group, a naphthyl group, etc.), and a group in which one hydrogen atom in the aromatic ring is substituted with an alkylene group (for example, arylalkyl groups such as a benzyl group, a phenethyl group, a 1-naphthyl methyl group, a 2-naphthyl methyl group, a 1-naphthyl ethyl group, and a 2-naphthyl ethyl group). The alkylene group (the alkyl chain in the arylalkyl group) preferably has 1 to 4 carbon atoms, more preferably has 1 to 2 carbon atoms, and particularly preferably has 1 carbon atom.

Examples of cyclic aliphatic hydrocarbon groups for R′²⁰¹ include an aliphatic hydrocarbon group containing a ring in the structure.

Examples of aliphatic hydrocarbon groups containing a ring in the structure include an alicyclic hydrocarbon group (a group in which one hydrogen atom has been removed from an aliphatic hydrocarbon ring), a group in which an alicyclic hydrocarbon group is bonded to the end of a linear or branched aliphatic hydrocarbon group, and a group in which an alicyclic hydrocarbon group is inserted midway along a linear or branched aliphatic hydrocarbon group.

The alicyclic hydrocarbon group preferably has 3 to 20 carbon atoms and more preferably has 3 to 12 carbon atoms.

The alicyclic hydrocarbon group may be a polycyclic group or a monocyclic group. The monocyclic alicyclic hydrocarbon group is preferably a group in which one or more hydrogen atoms have been removed from a monocycloalkane. The monocycloalkane preferably has 3 to 6 carbon atoms, and specific examples thereof include cyclopentane and cyclohexane. The polycyclic alicyclic hydrocarbon group is preferably a group in which one or more hydrogen atoms have been removed from a polycycloalkane, and the polycycloalkane preferably has 7 to 30 carbon atoms. Among these, the polycycloalkane is more preferably a polycycloalkane having a crosslinked ring type polycyclic framework such as adamantane, norbornane, isobornane, tricyclodecane, and tetracyclododecane; or a polycycloalkane having a fused ring type polycyclic framework such as a cyclic group having a steroid framework.

Among these, the cyclic aliphatic hydrocarbon group for R′²⁰¹ is preferably a group in which one or more hydrogen atoms have been removed from a monocycloalkane or polycycloalkane, more preferably a group in which one hydrogen atom has been removed from a polycycloalkane, particularly preferably an adamantyl group or a norbornyl group, and most preferably an adamantyl group.

The linear or branched aliphatic hydrocarbon group which may be bonded to an alicyclic hydrocarbon group has preferably 1 to 10 carbon atoms, more preferably has 1 to 6 carbon atoms, still more preferably has 1 to 4 carbon atoms, and particularly preferably has 1 to 3 carbon atoms.

The linear aliphatic hydrocarbon group is preferably a linear alkylene group, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group is preferably a branched alkylene group, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. The alkyl group in the alkylalkylene group is preferably a linear alkyl group having 1 to 5 carbon atoms.

In addition, the cyclic hydrocarbon group for R′²⁰¹ may contain a heteroatom such as a heterocycle. Specific examples thereof include lactone-containing cyclic groups represented by General Formulae (a2-r-1) to (a2-r-7), —SO₂-containing cyclic groups represented by General Formulae (b5-r-1) to (b5-r-4), and other heterocyclic groups represented by Chemical Formulae (r-hr-1) to (r-hr-16).

Examples of substituents in the cyclic group for R′²⁰¹ include an alkyl group, an alkoxy group, a halogen atom, a halogenated alkyl group, a hydroxyl group, a carbonyl group, and a nitro group.

The alkyl group as a substituent is preferably an alkyl group having 1 to 5 carbon atoms, and most preferably a methyl group, an ethyl group, a propyl group, an n-butyl group, or a tert-butyl group.

The alkoxy group as a substituent is preferably an alkoxy group having 1 to 5 carbon atoms, more preferably a methoxy group, an ethoxy group, an n-propoxy group, an iso-propoxy group, an n-butoxy group, or a tert-butoxy group, and most preferably a methoxy group or an ethoxy group.

The halogen atom as a substituent is preferably a fluorine atom.

Examples of halogenated alkyl groups as a substituent include an alkyl group having 1 to 5 carbon atoms, for example, a group in which some or all of the hydrogen atoms are substituted with the halogen atoms such as a methyl group, an ethyl group, a propyl group, an n-butyl group, and a tert-butyl group.

The carbonyl group as a substituent is a group that substitutes a methylene group (—CH₂—) constituting a cyclic hydrocarbon group.

Chain Alkyl Group which May have a Substituent:

The chain alkyl group for R′²⁰¹ may be either linear or branched.

The linear alkyl group preferably has 1 to 20 carbon atoms, more preferably has 1 to 15 carbon atoms, and most preferably has 1 to 10 carbon atoms.

The branched alkyl group preferably has 3 to 20 carbon atoms, more preferably has 3 to 15 carbon atoms, and most preferably has 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain Alkenyl Group which May have a Substituent:

The chain alkenyl group for R′²⁰¹ may be linear or branched, and preferably has 2 to 10 carbon atoms, more preferably has 2 to 5 carbon atoms, still more preferably has 2 to 4 carbon atoms, and particularly preferably has 3 carbon atoms. Examples of linear alkenyl groups include a vinyl group, a propenyl group (allyl group), and a butynyl group. Examples of branched alkenyl groups include a 1-methylvinyl group, a 2-methylvinyl group, a 1-methylpropenyl group, and a 2-methylpropenyl group.

Among the above examples, the chain alkenyl group is preferably a linear alkenyl group, more preferably a vinyl group or a propenyl group, and particularly preferably a vinyl group.

Examples of substituents in the chain alkyl group or alkenyl group for R′²⁰¹ include an alkoxy group, a halogen atom, a halogenated alkyl group, a hydroxyl group, a carbonyl group, a nitro group, an amino group, and the cyclic groups for R′²⁰¹.

Examples of cyclic groups which may have a substituent, chain alkyl groups which may have a substituent, or chain alkenyl groups which may have a substituent for R′²⁰¹ include, in addition to the above examples, the same groups as the acid-dissociable group represented by Formula (a1-r-2) as a cyclic group which may have a substituent or a chain alkyl group which may have a substituent.

Among these, R′²⁰¹ is preferably a cyclic group which may have a substituent and more preferably a cyclic hydrocarbon group which may have a substituent. More specifically, for example, a phenyl group, a naphthyl group, and a group in which one or more hydrogen atoms have been removed from a polycycloalkane; lactone-containing cyclic groups represented by General Formulae (a2-r-1) to (a2-r-7); and —SO₂-containing cyclic groups represented by General Formulae (b5-r-1) to (b5-r-4) are preferable.

In General Formulae (ca-1) to (ca-3), when R²⁰¹ to R²⁰³ and R²⁰⁶ to R²⁰⁷ are bonded to each other to form a ring together with a sulfur atom in the formula, they may be bonded via a heteroatom such as a sulfur atom, an oxygen atom, or a nitrogen atom, or a functional group such as a carbonyl group, —SO—, —SO₂—, —SO₃—, —COO—, —CONH— or —N(R_{N})—(R_{N} is an alkyl group having 1 to 5 carbon atoms). As the ring to be formed, one ring containing a sulfur atom in its ring framework in the formula is preferably a 3- to 10-membered ring and particularly preferably a 5- to 7-membered ring, including the sulfur atom. Specific examples of rings formed include a thiophene ring, a thiazole ring, a benzothiophene ring, a dibenzothiophene ring, a 9H-thioxanthene ring, a thioxanthone ring, a thianthrene ring, a phenoxathiin ring, a tetrahydrothiophenium ring, and a tetrahydrothiopyranium ring.

R²⁰⁸ to R²⁰⁹ are each independently a hydrogen atom or an alkyl group having 1 to 5 carbon atoms, and preferably a hydrogen atom or an alkyl group having 1 to 3 carbon atoms, when they are an alkyl group, they may be bonded to each other to form a ring.

R²¹⁰ is an aryl group which may have a substituent, an alkyl group which may have a substituent, alkenyl group which may have a substituent, or an —SO₂-containing cyclic group which may have a substituent.

Examples of aryl groups for R²¹⁰ include an unsubstituted aryl group having 6 to 20 carbon atoms, and a phenyl group and a naphthyl group are preferable.

The alkyl group for R²¹⁰ is preferably a chain or cyclic alkyl group having 1 to 30 carbon atoms.

The alkenyl group for R²¹⁰ preferably has 2 to 10 carbon atoms.

The —SO₂-containing cyclic group which may have a substituent for R²¹⁰ is preferably a “—SO₂-containing polycyclic group” and more preferably the group represented by General Formula (b5-r-1).

Specific examples of cations represented by Formula (ca-1) are shown below.

Specific examples of preferable cations represented by Formula (ca-1) include cations represented by the following chemical formulae.

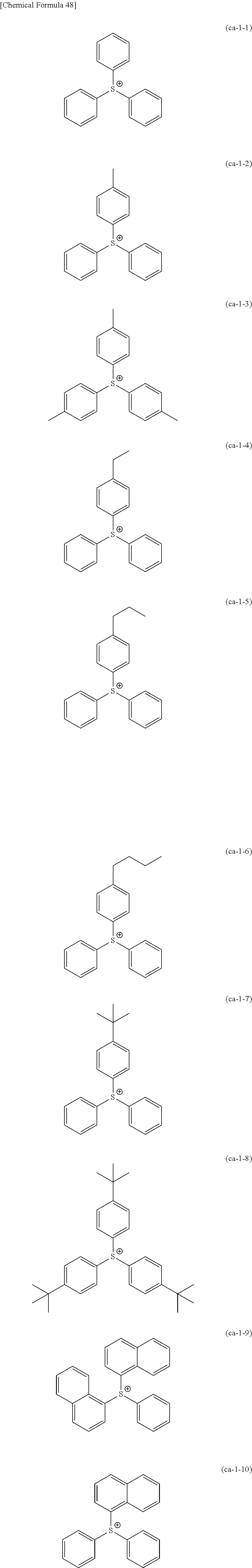

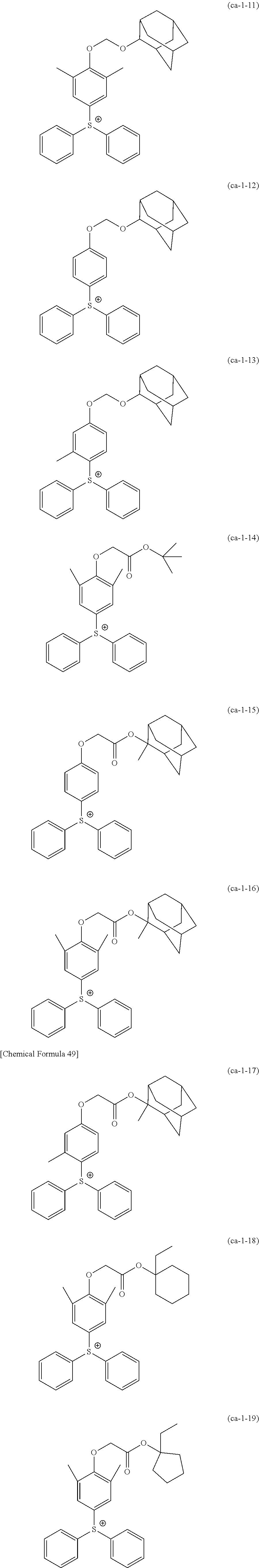

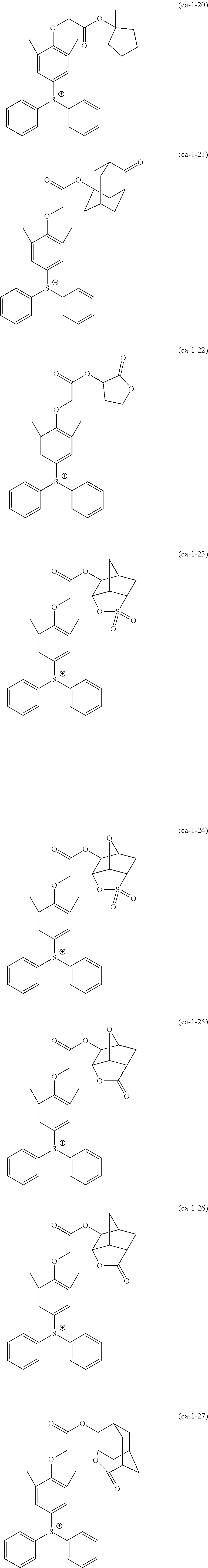

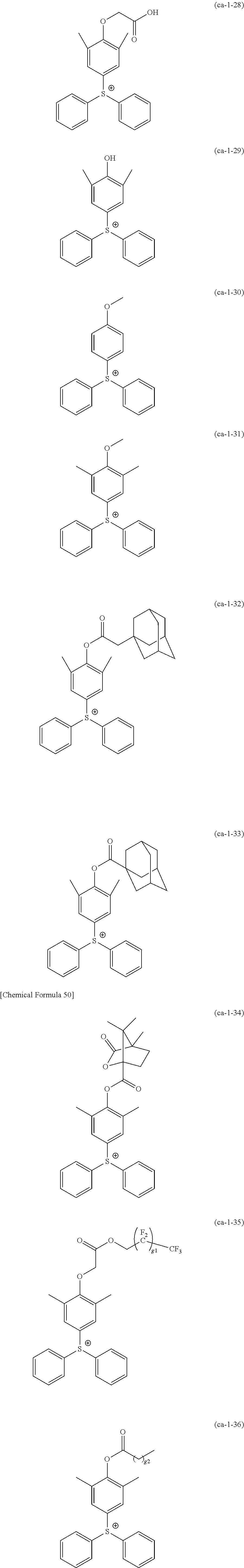

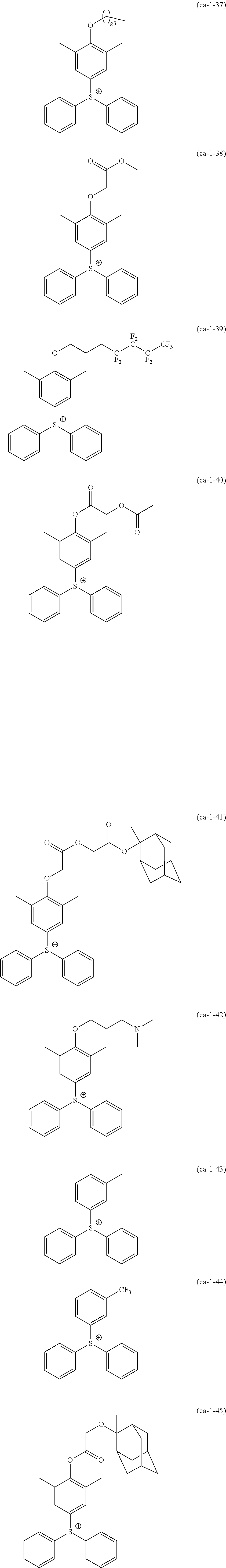

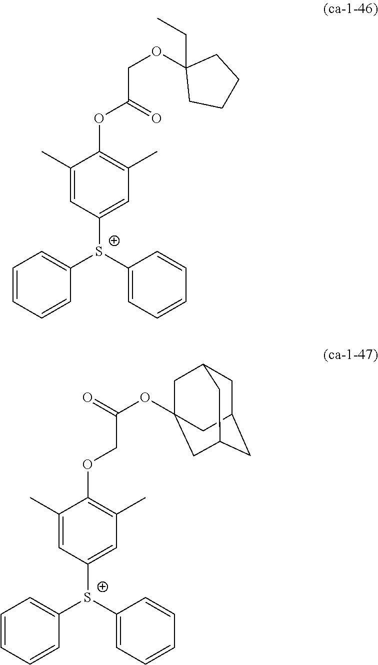 [SMILES: CCC1(OCC(=O)Oc2c(C)cc([S+](c3ccccc3)c3ccccc3)cc2C)CCCC1 ; Cc1cc([S+](c2ccccc2)c2ccccc2)cc(C)c1OCC(=O)OC12CC3CC(CC(C3)C1)C2]

[in the formula, g1, g2, and g3 are the number of repetitions, g1 is an integer of 1 to 5, g2 is an integer of 0 to 20, and g3 is an integer of 0 to 20].

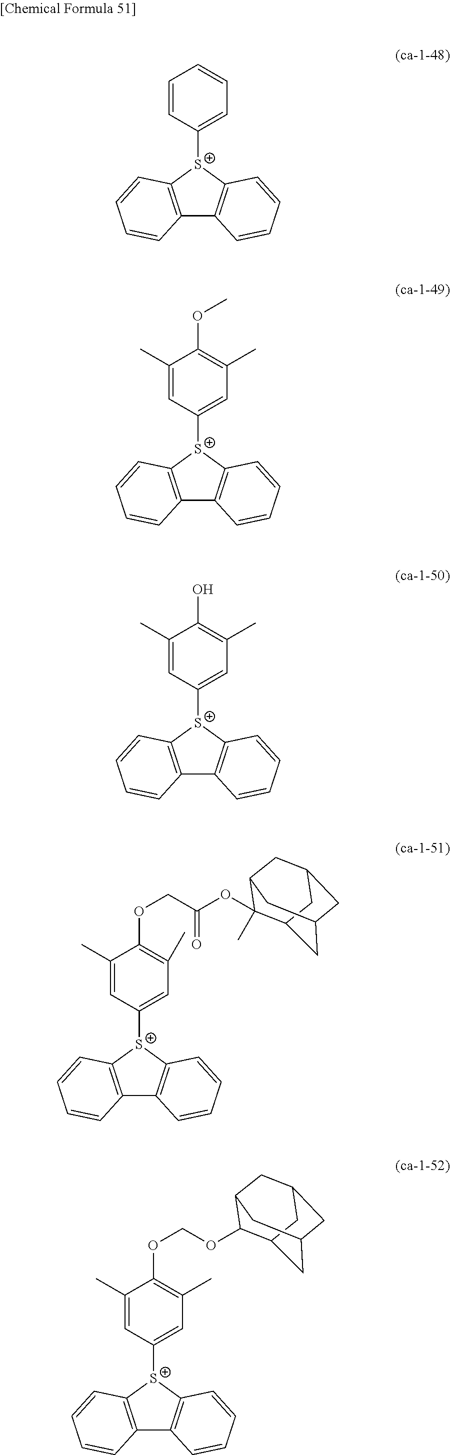 [SMILES: c1ccc(-[s+]2c3ccccc3c3ccccc32)cc1 ; COc1c(C)cc(-[s+]2c3ccccc3c3ccccc32)cc1C ; Cc1cc(-[s+]2c3ccccc3c3ccccc32)cc(C)c1O ; Cc1cc(-[s+]2c3ccccc3c3ccccc32)cc(C)c1OCC(=O)OC1(C)C2CC3CC(C2)CC1C3 ; Cc1cc(-[s+]2c3ccccc3c3ccccc32)cc(C)c1OCOC1C2CC3CC(C2)CC1C3]

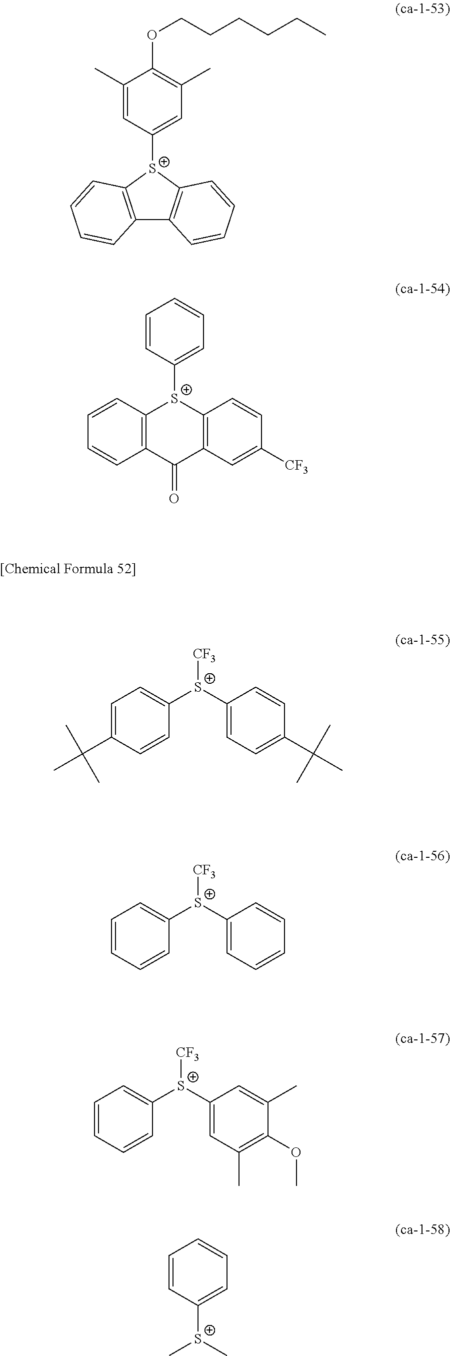

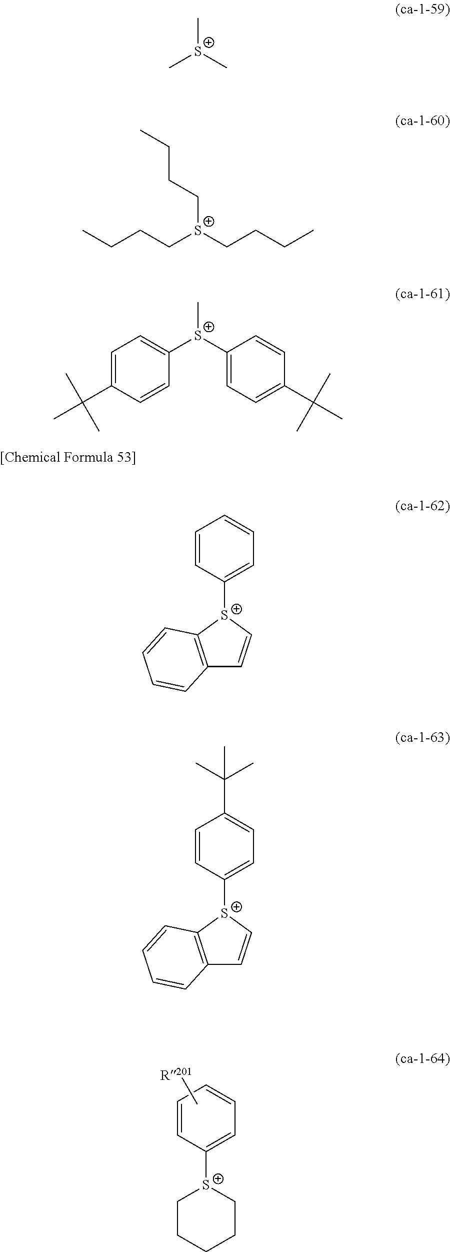

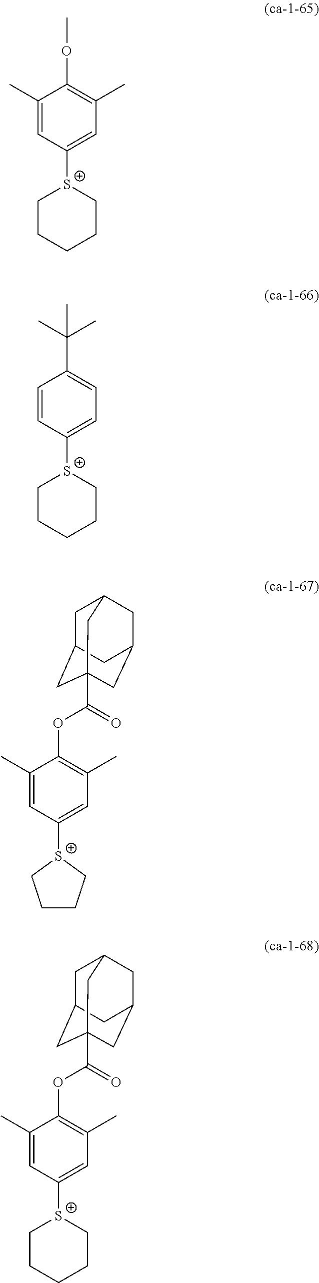

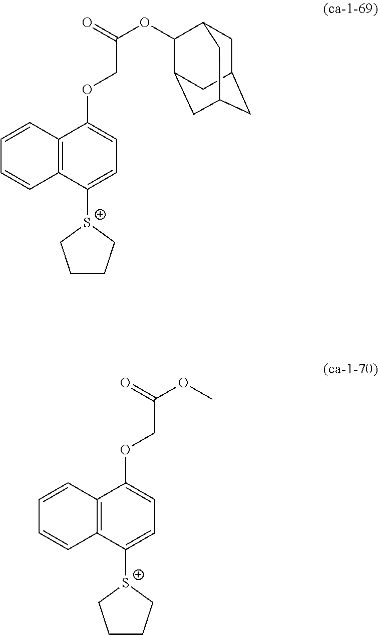 [SMILES: O=C(COc1ccc([S+]2CCCC2)c2ccccc12)OC1C2CC3CC(C2)CC1C3 ; COC(=O)COc1ccc([S+]2CCCC2)c2ccccc12]

[in the formula, R″²⁰¹ is a hydrogen atom or a substituent, and the substituents are the same as those exemplified as the substituents that R²⁰¹ to R²⁰⁷ and R²¹⁰ to R²¹² may have].

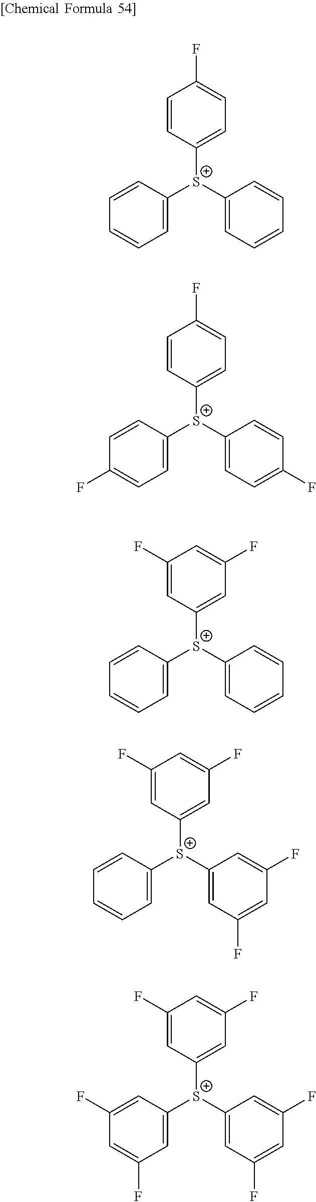

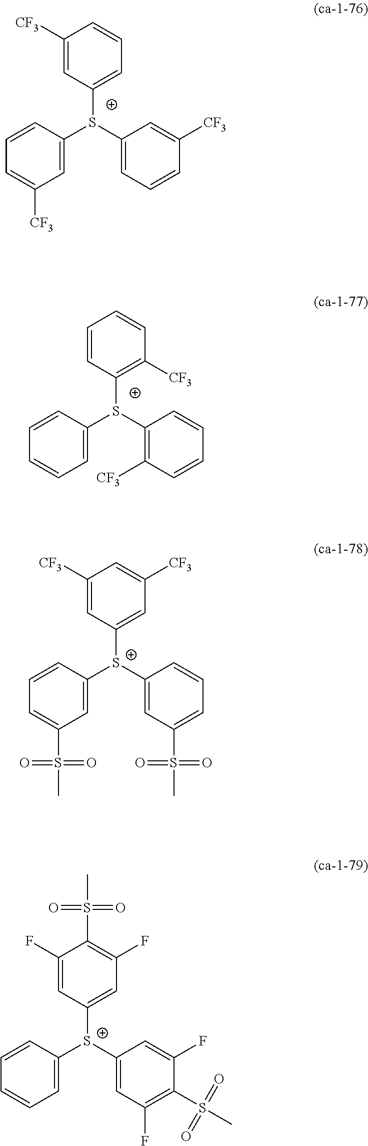

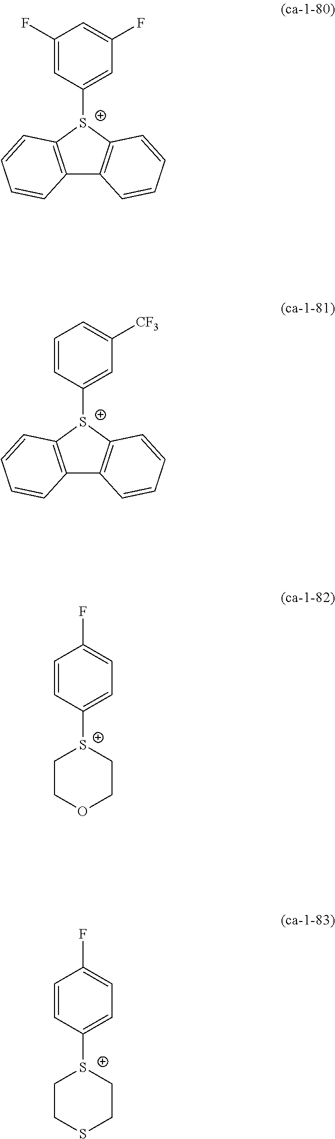

Specific examples of preferable cations represented by Formula (ca-2) include diphenyliodonium cations and bis(4-tert-butylphenyl)iodonium cations.

Specific examples of preferable cations represented by Formula (ca-3) include cations represented by the following Formulae (ca-3-1) to (ca-3-6).

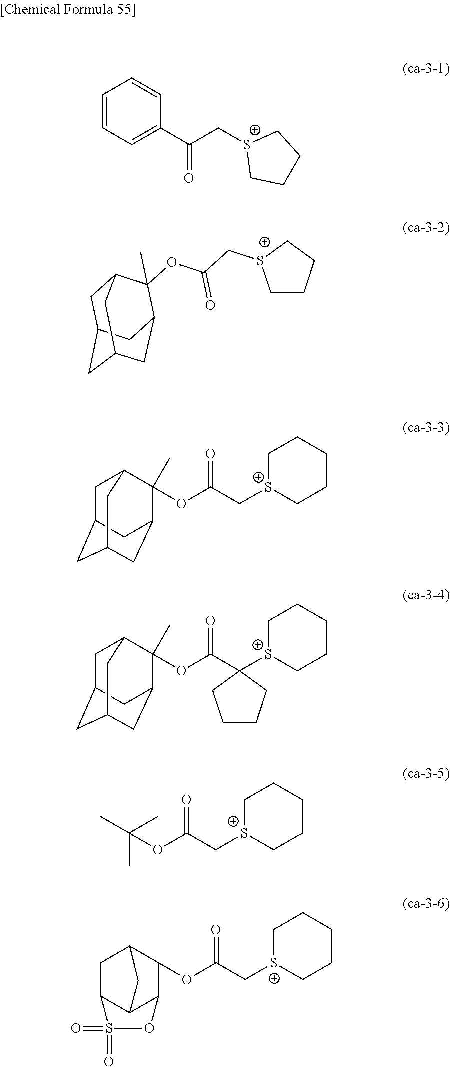 [SMILES: O=C(C[S+]1CCCC1)c1ccccc1 ; CC1(OC(=O)C[S+]2CCCC2)C2CC3CC(C2)CC1C3 ; CC1(OC(=O)C[S+]2CCCCC2)C2CC3CC(C2)CC1C3 ; CC1(OC(=O)C2([S+]3CCCCC3)CCCC2)C2CC3CC(C2)CC1C3 ; CC(C)(C)OC(=O)C[S+]1CCCCC1 ; O=C(C[S+]1CCCCC1)OC1C2CC3C1OS(=O)(=O)C3C2]

In the resist composition of the present embodiment, the components (B) may be used alone or two or more thereof may be used in combination.

When the resist composition contains the component (B), in the resist composition, the content of the component (B) with respect to 100 parts by mass of the component (A) is preferably less than 50 parts by mass, more preferably 10 to 40 parts by mass, and still more preferably 20 to 40 parts by mass.

When the content of the component (B) is within the above preferable range, pattern formation is sufficiently performed. In addition, when each component of the resist composition is dissolved in an organic solvent, this is preferable because a uniform solution is easily obtained and the storage stability of the resist composition is improved.

### <<Base Component (D)>>

The resist composition of the present embodiment further contains a base component (hereinafter referred to as a “component (D)”) that traps (that is, controls diffusion of an acid) an acid generated upon exposure. The component (D) functions as a quencher (acid diffusion control agent) that traps an acid generated in the resist composition upon exposure.

The resist composition of the present embodiment contains, as the component (D), at least a compound (D0) represented by the following General Formula (d0) (component (D0)). Examples of components (D) other than the component (D0) include a photodecomposable base (D1) (hereinafter referred to as a “component (D1)”) that decomposes upon exposure and loses its ability to control acid diffusion and a nitrogen-containing organic compound (D2) (hereinafter referred to as a “component (D2)”) that does not correspond to the component (D1). Among these, the photodecomposable base (component (D1)) is preferable because it more easily improves roughness reduction. In addition, when the resist composition contains the component (D1), it becomes easier to improve properties of increasing sensitivity and reducing the occurrence of coating defects.

### Component (D0)

The component (D0) is a compound represented by the following General Formula (d0).

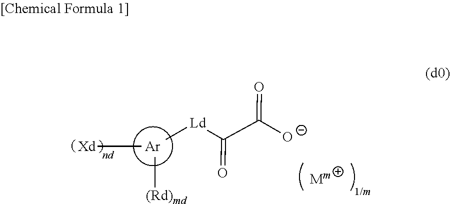

[in the formula, Ar is an aromatic ring; Xd is an iodine atom, a fluorine atom, a bromine atom or a fluorinated alkyl group; Rd is a substituent; nd is an integer of 1 or more as long as the valence allows, and md is an integer of 0 or more as long as the valence allows; Ld is a single bond or a divalent linking group; when nd is an integer of 2 or more, a plurality of Xd's may be the same as or different from each other; when md is an integer of 2 or more, a plurality of Rd's may be the same as or different from each other; and m is an integer of 1 or more, and M^{m+} is an m-valent cation].

### (Anion Moiety of Component (D0))

In Formula (d0), Ar is an aromatic ring. The aromatic ring for Ar is not particularly limited as long as it is a cyclic conjugated system having 4n+2 π electrons, and may be monocyclic or polycyclic. The aromatic ring preferably has 5 to 30 carbon atoms, more preferably has 5 to 20 carbon atoms, still more preferably has 6 to 15 carbon atoms, and particularly preferably 6 to 12 carbon atoms.

Specific examples of aromatic rings include aromatic hydrocarbon rings such as benzene, naphthalene, anthracene, and phenanthrene.

Among these, Ar is preferably a benzene ring, a naphthalene ring or an anthracene ring, more preferably a benzene ring or a naphthalene ring, and still more preferably a benzene ring.

In Formula (d0), Xd is an iodine atom, a fluorine atom, a bromine atom or a fluorinated alkyl group. In order to increase the sensitivity, Xd is preferably an iodine atom.

In Formula (d0), Rd is a substituent. Examples of substituents for Rd include an alkyl group, an alkoxy group, a hydroxy group, a nitro group, a chlorine atom, —C(═O)—O—Rd⁰¹, —O—C(═O)—Rd⁰², an amino group, and —NH—C(═O)—Rd⁰².

Rd⁰¹ is an alkyl group, and preferably a linear or branched alkyl group having 1 to 5 carbon atoms.

Rd⁰² is a hydrocarbon group which may have a substituent. The hydrocarbon group for Rd⁰² is preferably an alkyl group or an aromatic hydrocarbon group.

The alkyl group for Rd⁰² is preferably a linear or branched alkyl group having 1 to 5 carbon atoms.

The aromatic hydrocarbon group for Rd⁰² is preferably a phenyl group or a naphthyl group, and more preferably a phenyl group.

Examples of substituents that the hydrocarbon group for Rd⁰² may have include an iodine atom and a fluorine atom.

The alkyl group as a substituent for Rd is preferably an alkyl group having 1 to 5 carbon atoms, and most preferably a methyl group, an ethyl group, a propyl group, an n-butyl group, or a tert-butyl group.

The alkoxy group as a substituent for Rd is preferably an alkoxy group having 1 to 5 carbon atoms, more preferably a methoxy group, an ethoxy group, an n-propoxy group, an iso-propoxy group, an n-butoxy group, or a tert-butoxy group, and most preferably a methoxy group or an ethoxy group.

Among these, in order to increase the sensitivity and improve CDU, Rd is preferably a hydroxy group.

In Formula (d0), nd is an integer of 1 or more as long as the valence allows. In order to achieve both high sensitivity and solubility of the resist composition in a resist solvent, nd is preferably an integer of 1 to 5, more preferably an integer of 1 to 3, still more preferably 2 or 3, and particularly preferably 3.

When nd is an integer of 2 or more, a plurality of Xd's may be the same as or different from each other.

In Formula (d0), md is an integer of 0 or more as long as the valence allows. In consideration of the solubility of the resist composition in a resist solvent, md is preferably an integer of 0 to 3, more preferably an integer of 0 to 2, still more preferably 0 or 1, and particularly preferably 0.

When md is an integer of 2 or more, a plurality of Rd's may be the same as or different from each other.

In Formula (d0), Ld is a single bond or a divalent linking group.

Examples of divalent linking groups for Ld include the same divalent linking groups as for Ya^{x1} in General Formula (a10-1).

The divalent linking group for Ld is preferably —C(═O)—O—, —O—C(═O)—, —O—, —C(═O)—, —NH—, —S—, a linear or branched aliphatic hydrocarbon group, or a combination thereof. The linear or branched aliphatic hydrocarbon group is preferably a linear or branched alkylene group having 1 to 6 carbon atoms or a linear or branched alkenylene group having 2 to 6 carbon atoms. The divalent linking group for Ld is more preferably —C(═O)—O—, —O—C(═O)—, —O—, or —C(═O)—.

In order to improve the solubility in a development solution, Ld is preferably a single bond.

Specific examples of anion moieties of the component (D0) are shown below.

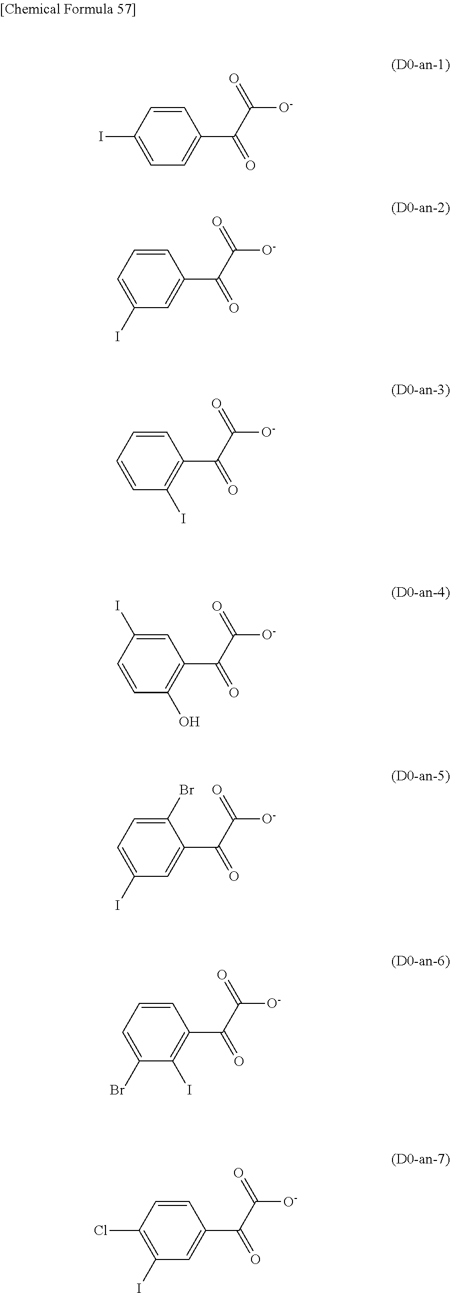

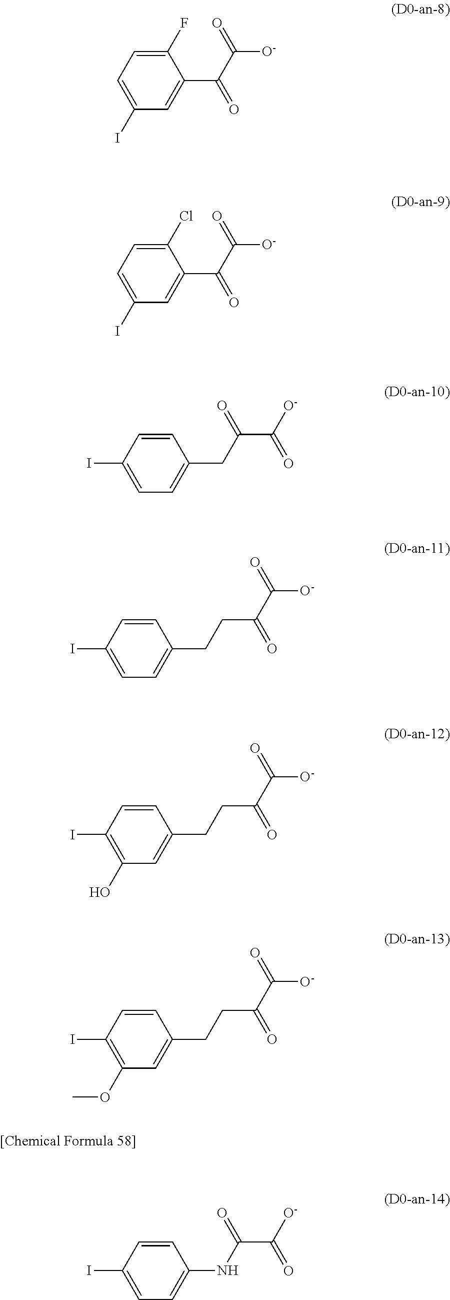

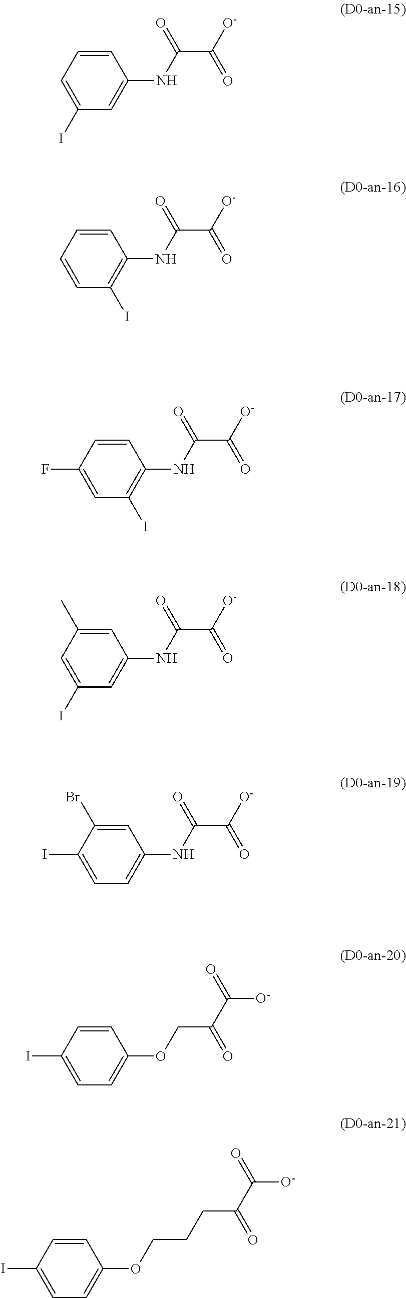

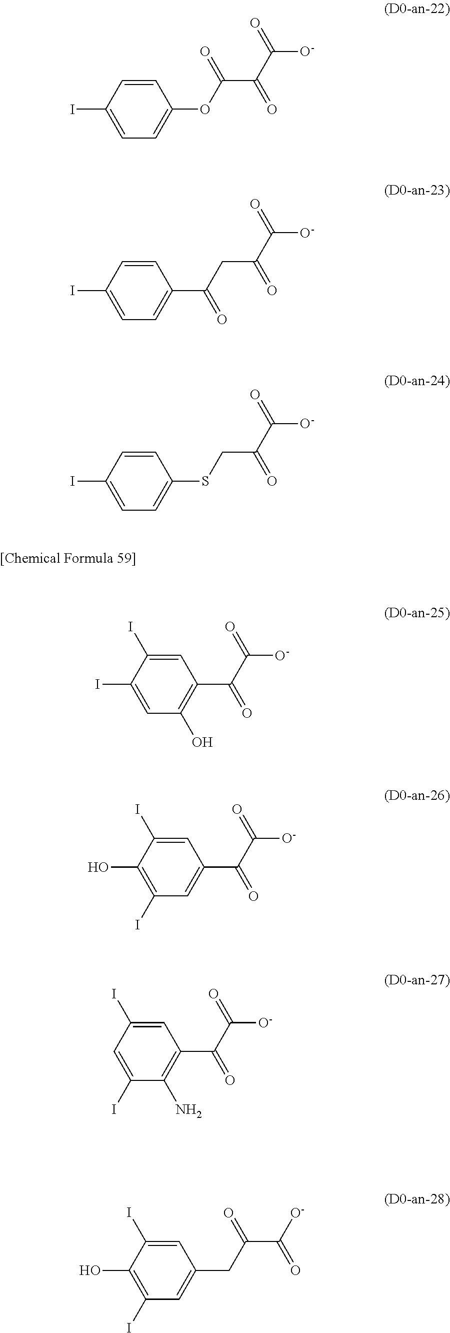

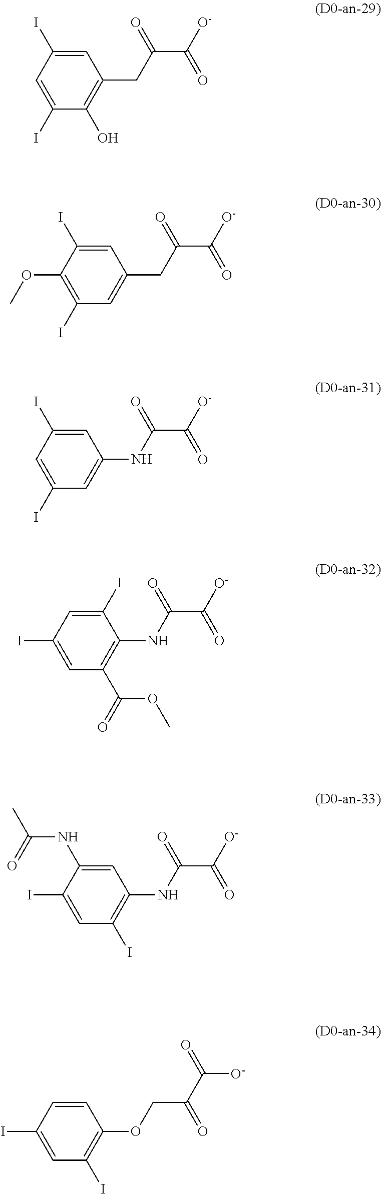 [SMILES: O=C([O-])C(=O)Cc1cc(I)cc(I)c1O ; COc1c(I)cc(CC(=O)C(=O)[O-])cc1I ; O=C([O-])C(=O)Nc1cc(I)cc(I)c1 ; COC(=O)c1cc(I)cc(I)c1NC(=O)C(=O)[O-] ; CC(=O)Nc1cc(NC(=O)C(=O)[O-])c(I)cc1I ; O=C([O-])C(=O)COc1ccc(I)cc1I]

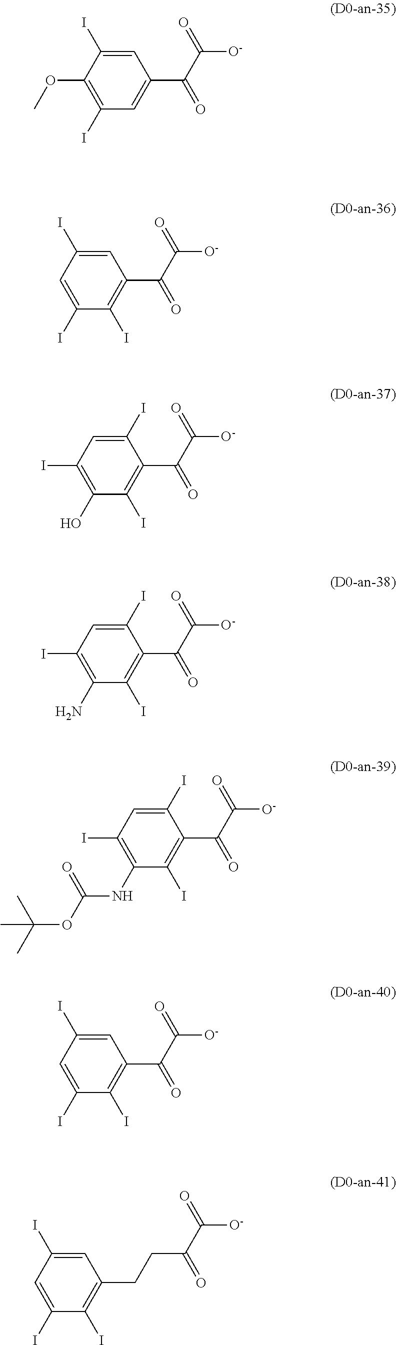

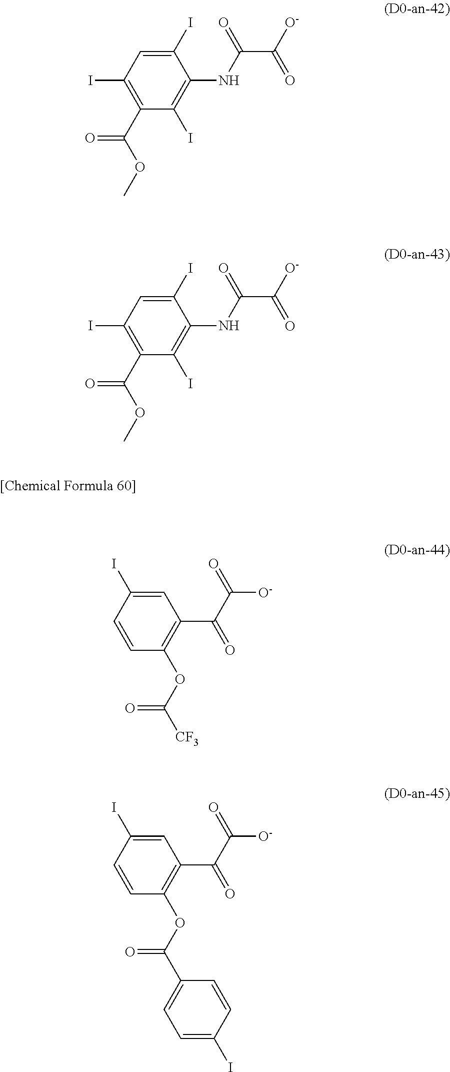 [SMILES: COC(=O)c1c(I)cc(I)c(NC(=O)C(=O)[O-])c1I ; COC(=O)c1c(I)cc(I)c(NC(=O)C(=O)[O-])c1I ; O=C([O-])C(=O)c1cc(I)ccc1OC(=O)C(F)(F)F ; O=C([O-])C(=O)c1cc(I)ccc1OC(=O)c1ccc(I)cc1]

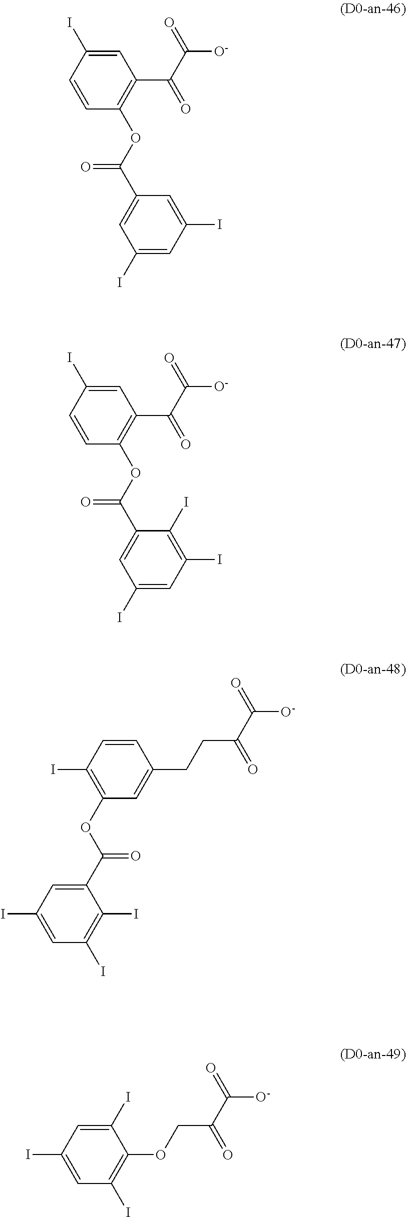 [SMILES: O=C([O-])C(=O)c1cc(I)ccc1OC(=O)c1cc(I)cc(I)c1 ; O=C([O-])C(=O)c1cc(I)ccc1OC(=O)c1cc(I)cc(I)c1I ; O=C([O-])C(=O)CCc1ccc(I)c(OC(=O)c2cc(I)cc(I)c2I)c1 ; O=C([O-])C(=O)COc1c(I)cc(I)cc1I]

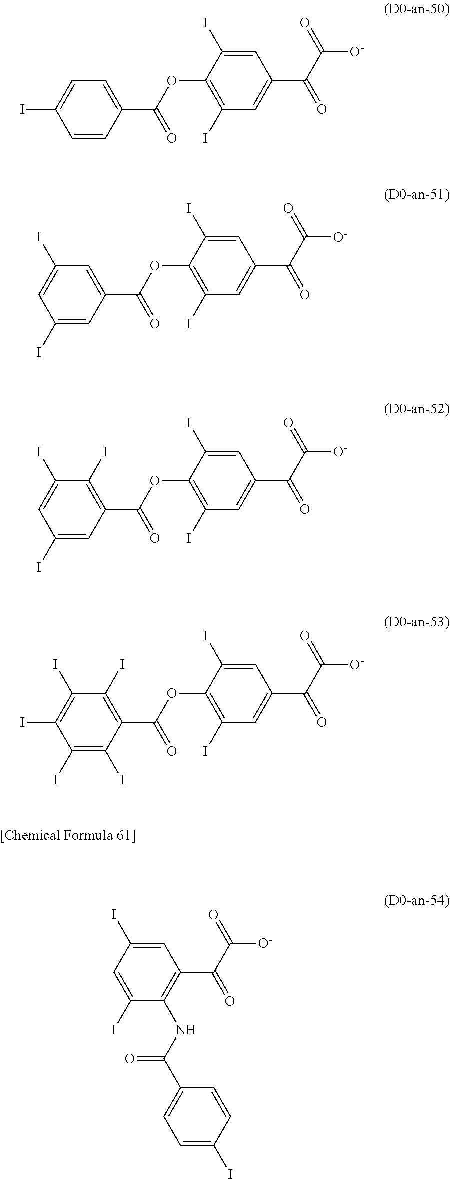 [SMILES: O=C([O-])C(=O)c1cc(I)c(OC(=O)c2ccc(I)cc2)c(I)c1 ; O=C([O-])C(=O)c1cc(I)c(OC(=O)c2cc(I)cc(I)c2)c(I)c1 ; O=C([O-])C(=O)c1cc(I)c(OC(=O)c2cc(I)cc(I)c2I)c(I)c1 ; O=C([O-])C(=O)c1cc(I)c(OC(=O)c2c(I)c(I)c(I)c(I)c2I)c(I)c1 ; O=C([O-])C(=O)c1cc(I)cc(I)c1NC(=O)c1ccc(I)cc1]

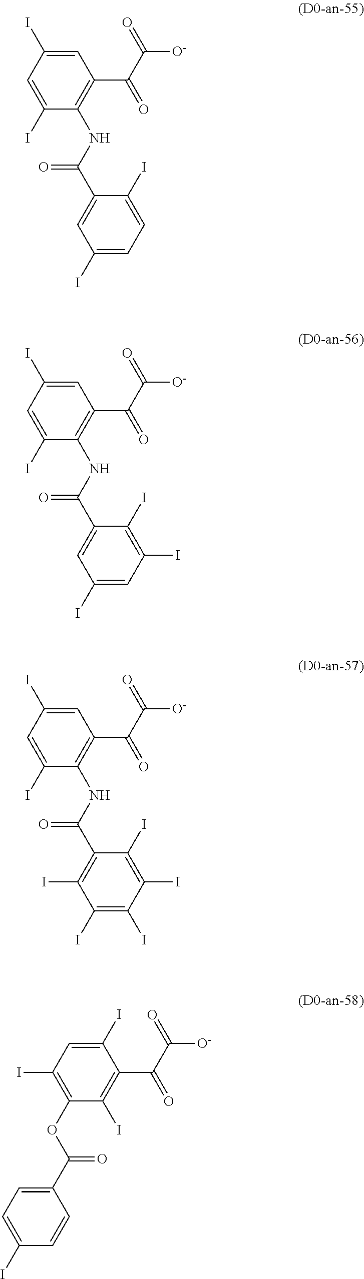 [SMILES: O=C([O-])C(=O)c1cc(I)cc(I)c1NC(=O)c1cc(I)ccc1I ; O=C([O-])C(=O)c1cc(I)cc(I)c1NC(=O)c1cc(I)cc(I)c1I ; O=C([O-])C(=O)c1cc(I)cc(I)c1NC(=O)c1c(I)c(I)c(I)c(I)c1I ; O=C([O-])C(=O)c1c(I)cc(I)c(OC(=O)c2ccc(I)cc2)c1I]

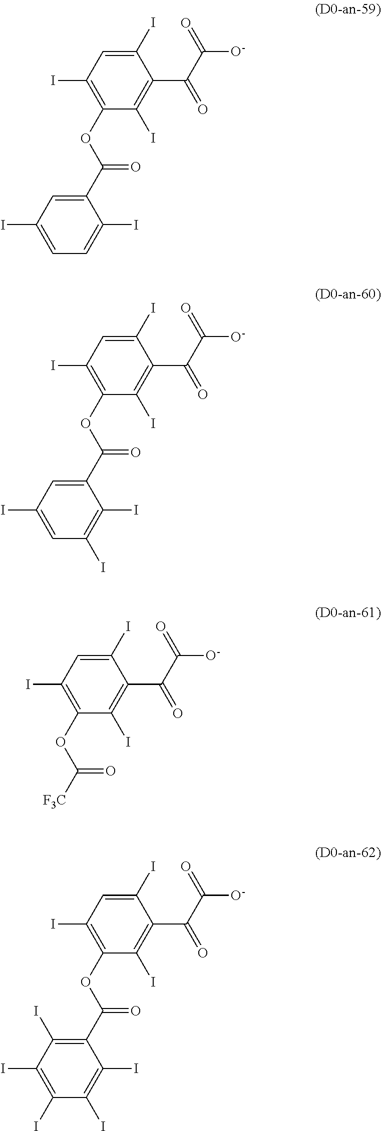 [SMILES: O=C([O-])C(=O)c1c(I)cc(I)c(OC(=O)c2cc(I)ccc2I)c1I ; O=C([O-])C(=O)c1c(I)cc(I)c(OC(=O)c2cc(I)cc(I)c2I)c1I ; O=C([O-])C(=O)c1c(I)cc(I)c(OC(=O)C(F)(F)F)c1I ; O=C([O-])C(=O)c1c(I)cc(I)c(OC(=O)c2c(I)c(I)c(I)c(I)c2I)c1I]

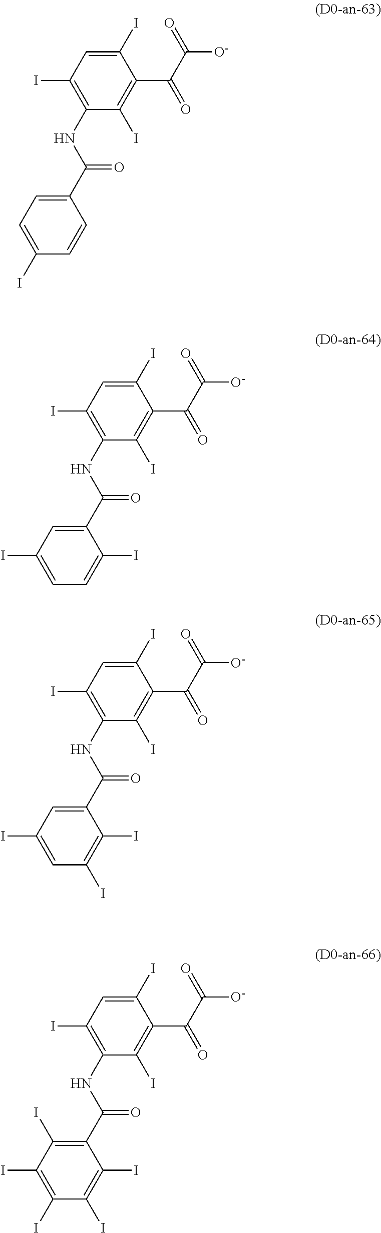 [SMILES: O=C([O-])C(=O)c1c(I)cc(I)c(NC(=O)c2ccc(I)cc2)c1I ; O=C([O-])C(=O)c1c(I)cc(I)c(NC(=O)c2cc(I)ccc2I)c1I ; O=C([O-])C(=O)c1c(I)cc(I)c(NC(=O)c2cc(I)cc(I)c2I)c1I ; O=C([O-])C(=O)c1c(I)cc(I)c(NC(=O)c2c(I)c(I)c(I)c(I)c2I)c1I]

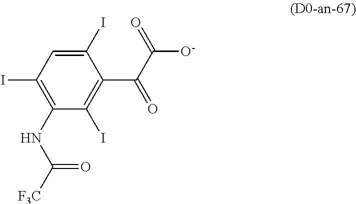 [SMILES: O=C([O-])C(=O)c1c(I)cc(I)c(NC(=O)C(F)(F)F)c1I]

### (Cation Moiety of Component (D0))

In Formula (d0), M′^{m+} is an m-valent cation. The cation for M′^{m+} is preferably an onium cation, more preferably a sulfonium cation or an iodonium cation, still more preferably a cation represented by each of General Formulae (ca-1) to (ca-3), and particularly preferably the organic cation represented by General Formula (ca-1).

Among these, in order to improve the sensitivity and CDU, the cation for M′^{m+} is the cation represented by General Formula (ca-1), in which at least one of R²⁰¹ to R²⁰³ is preferably an aryl group having a fluorine atom or an aryl group having a fluorinated alkyl group.

Specific examples of components (D0) are shown below.

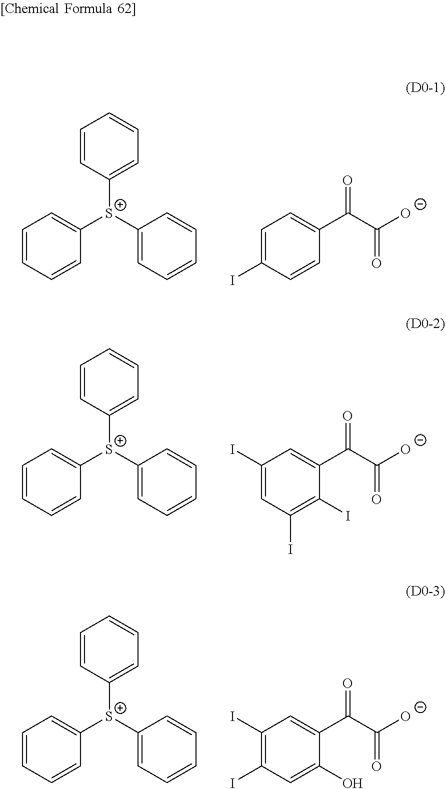

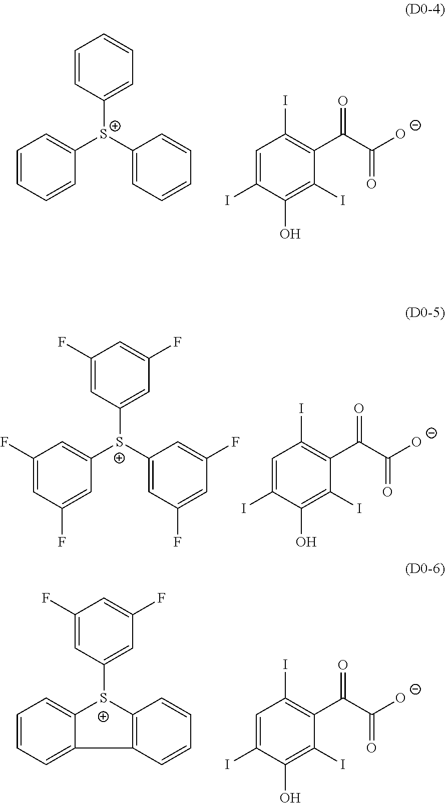 [SMILES: c1ccc([S+](c2ccccc2)c2ccccc2)cc1 ; O=C([O-])C(=O)c1c(I)cc(I)c(O)c1I ; Fc1cc(F)cc([S+](c2cc(F)cc(F)c2)c2cc(F)cc(F)c2)c1 ; O=C([O-])C(=O)c1c(I)cc(I)c(O)c1I ; Fc1cc(F)cc(-[s+]2c3ccccc3c3ccccc32)c1 ; O=C([O-])C(=O)c1c(I)cc(I)c(O)c1I]

In the resist composition of the present embodiment, the components (D0) may be used alone or two or more thereof may be used in combination.

In the resist composition of the present embodiment, the content of the component (D0) with respect to 100 parts by mass of the component (A) is preferably 1 to 80 parts by mass, more preferably 3 to 60 parts by mass, still more preferably 3 to 40 parts by mass, and particularly preferably 3 to 30 parts by mass.

When the content of the component (D0) is equal to or larger than the lower limit value in the preferable range, lithography properties such as CDU are more likely to be improved during resist pattern formation. On the other hand, when the content of the component (D0) is equal to or smaller than the upper limit value in the preferable range, it is easier to maintain favorable sensitivity and to improve the solubility in a development solution.

### Component (D1)

When the resist composition contains the component (D1), it is possible to further improve the contrast between the exposed part and the unexposed part of the resist film when a resist pattern is formed.

The component (D1) is not particularly limited as long as it is decomposed upon exposure and loses its ability to control acid diffusion, and is preferably one or more compounds selected from the group consisting of a compound represented by the following General Formula (d1-1) (hereinafter referred to as a “component (d1-1)”), a compound represented by the following General Formula (d1-2) (hereinafter referred to as a “component (d1-2)”) and a compound represented by the following General Formula (d1-3) (hereinafter referred to as a “component (d1-3)”).

The components (d1-1) to (d1-3) do not function as a quencher in the exposed part of the resist film because they decompose and lose their ability to control acid diffusion (basicity), but function as a quencher in the unexposed part of the resist film.

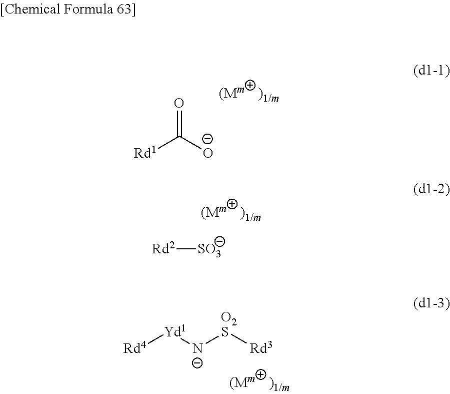

[in the formula, Rd¹ to Rd⁴ are a cyclic group which may have a substituent, a chain alkyl group which may have a substituent, or a chain alkenyl group which may have a substituent, here, in Rd² in Formula (d1-2), the carbon atom adjacent to the S atom has no fluorine atom bonded thereto, Yd¹ is a single bond or a divalent linking group, m is an integer of 1 or more, and M′^{m+}'s are each independently an m-valent organic cation].
{Component (d1-1)}

### Anion Moiety

In Formula (d1-1), Rd¹ is a cyclic group which may have a substituent, a chain alkyl group which may have a substituent, or a chain alkenyl group which may have a substituent, and examples thereof include the same groups as for R′²⁰¹.

Among these, Rd¹ is preferably an aromatic hydrocarbon group which may have a substituent, an aliphatic cyclic group which may have a substituent, or a chain alkyl group which may have a substituent. Examples of substituents that these groups may have include a hydroxyl group, an oxo group, an alkyl group, an aryl group, a fluorine atom, a fluorinated alkyl group, the lactone-containing cyclic groups represented by General Formulae (a2-r-1) to (a2-r-7), an ether bond, an ester bond, and a combination thereof. When an ether bond or an ester bond is contained as a substituent, an intervening alkylene group may be provided, and the substituent in this case is preferably a linking group represented by each of Formulae (y-a1-1) to (y-a1-5). Here, when the aromatic hydrocarbon group, the aliphatic cyclic group, or the chain alkyl group for Rd¹ has, as a substituent, a linking group represented by each of General Formulae (y-a1-1) to (y-a1-7), in General Formulae (y-a1-1) to (y-a1-7), V′¹⁰¹ in General Formulae (y-a1-1) to (y-a1-7) bonds to a carbon atom constituting the aromatic hydrocarbon group, the aliphatic cyclic group, or the chain alkyl group for Rd¹ in Formula (d3-1).

Preferable examples of aromatic hydrocarbon groups include a phenyl group, a naphthyl group, and a polycyclic structure containing a bicyclooctane framework (a polycyclic structure composed of a bicyclooctane framework and another ring structure).

The aliphatic cyclic group is more preferably a group in which one or more hydrogen atoms have been removed from a polycycloalkane such as adamantane, norbornane, isobornane, tricyclodecane, or tetracyclododecane.

The chain alkyl group preferably has 1 to 10 carbon atoms, and specific examples thereof include linear alkyl groups such as a methyl group, an ethyl group, a propyl group, a butyl group, a pentyl group, a hexyl group, a heptyl group, an octyl group, a nonyl group, and a decyl group; and branched alkyl groups such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

When the chain alkyl group is a fluorinated alkyl group having a fluorine atom or a fluorinated alkyl group as a substituent, the fluorinated alkyl group preferably has 1 to 11 carbon atoms, more preferably has 1 to 8 carbon atoms, and still more preferably 1 to 4 carbon atoms. The fluorinated alkyl group may contain an atom other than a fluorine atom. Examples of atoms other than a fluorine atom include an oxygen atom, a sulfur atom, and a nitrogen atom.

Preferable specific examples of anion moieties of the component (d1-1) are shown below.

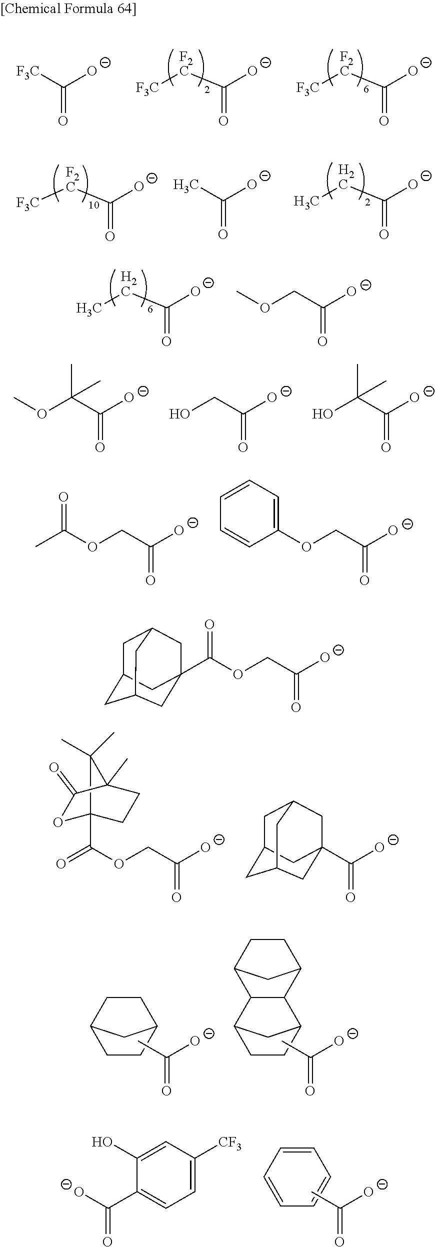

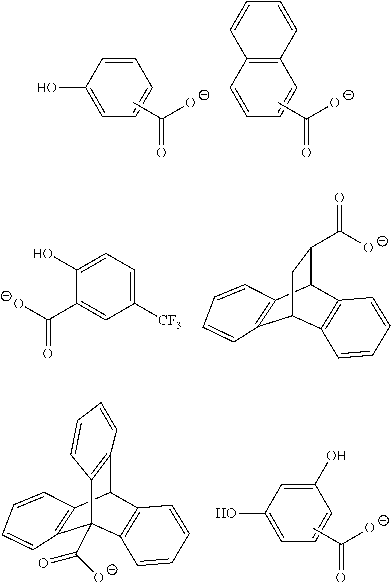

### Cation Moiety

In Formula (d1-1), M′^{m+} is an m-valent organic cation.

Preferable examples of organic cations for M^{m+} include the same cations represented by General Formulae (ca-1) to (ca-5), and the cation represented by General Formula (ca-1) is more preferable, and the cation represented by each of Formulae (ca-1-1) to (ca-1-70) is still more preferable.

Components (d1-1) may be used alone or two or more thereof may be used in combination.

Components (d1-1) may be used alone or two or more thereof may be used in combination.

{Component (d1-2)}

### Anion Moiety

In Formula (d1-2), Rd² is a cyclic group which may have a substituent, a chain alkyl group which may have a substituent, or a chain alkenyl group which may have a substituent, and examples thereof include the same groups as for R′²⁰¹

Here, in Rd², the carbon atom adjacent to the S atom has no fluorine atom bonded thereto (is not substituted with fluorine). Accordingly, the anion of the component (d1-2) becomes an appropriately weak acid anion, and the quenching ability of the component (D) is improved.

Rd² is preferably a chain alkyl group which may have a substituent or an aliphatic cyclic group which may have a substituent, and more preferably an aliphatic cyclic group which may have a substituent.

The chain alkyl group preferably has 1 to 10 carbon atoms and more preferably has 3 to 10 carbon atoms.

The aliphatic cyclic group is more preferably a group in which one or more hydrogen atoms have been removed from adamantane, norbornane, isobornane, tricyclodecane, tetracyclododecane or the like (which may have a substituent); or a group in which one or more hydrogen atoms have been removed from camphor.

The hydrocarbon group for Rd² may have a substituent, and examples of substituents include the same substituents that the hydrocarbon group (aromatic hydrocarbon group, aliphatic cyclic group, chain alkyl group) for Rd¹ in Formula (d1-1) may have.

Preferable specific examples of anion moieties of the component (d1-2) are shown below.

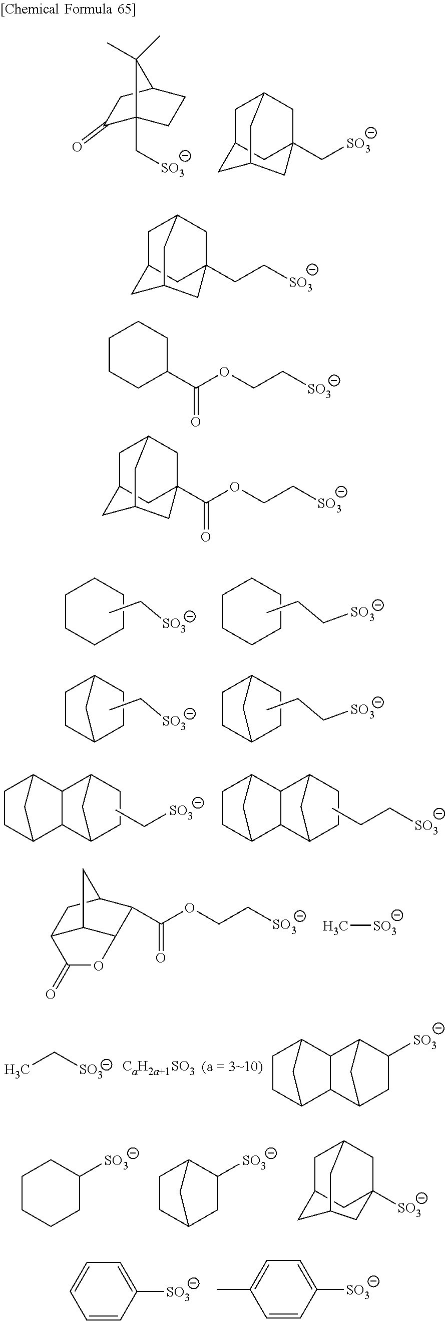

### Cation Moiety

In Formula (d1-2), M^{m+} is an m-valent organic cation, and is the same as M^{m+} in Formula (d1-1).

Components (d1-2) may be used alone or two or more thereof may be used in combination.

{Component (d1-3)}

### Anion Moiety

In Formula (d1-3), Rd³ is a cyclic group which may have a substituent, a chain alkyl group which may have a substituent, or a chain alkenyl group which may have a substituent, and examples thereof include the same as those for R′²⁰¹, and Rd³ is preferably a fluorine atom-containing cyclic group, a chain alkyl group, or a chain alkenyl group. Among these, a fluorinated alkyl group is preferable, and the same fluorinated alkyl group as for Rd¹ is more preferable.

In Formula (d1-3), Rd⁴ is a cyclic group which may have a substituent, a chain alkyl group which may have a substituent, or a chain alkenyl group which may have a substituent, and examples thereof include the same as those for R′²⁰¹.

Among these, an alkyl group, alkoxy group, alkenyl group, or cyclic group which may have a substituent is preferable.

The alkyl group for Rd⁴ is preferably a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. Some hydrogen atoms in the alkyl group for Rd⁴ may be substituted with a hydroxyl group, a cyano group or the like.

The alkoxy group for Rd⁴ is preferably an alkoxy group having 1 to 5 carbon atoms, and specific examples of alkoxy groups having 1 to 5 carbon atoms include a methoxy group, an ethoxy group, an n-propoxy group, an iso-propoxy group, an n-butoxy group, and a tert-butoxy group. Among these, a methoxy group or an ethoxy group is preferable.

Examples of alkenyl groups for Rd⁴ include the same alkenyl groups as for R′²⁰¹, and a vinyl group, a propenyl group (allyl group), a 1-methylpropenyl group, and a 2-methylpropenyl group are preferable. These groups may further contain, as a substituent, an alkyl group having 1 to 5 carbon atoms or a halogenated alkyl group having 1 to 5 carbon atoms.

Examples of cyclic groups for Rd⁴ include the same cyclic groups as for R′²⁰¹ and an alicyclic group in which one or more hydrogen atoms have been removed from a cycloalkane such as cyclopentane, cyclohexane, adamantane, norbornane, isobornane, tricyclodecane, or tetracyclododecane, or an aromatic group such as a phenyl group and a naphthyl group is preferable. When Rd⁴ is an alicyclic group, the resist composition is easily dissolved in an organic solvent and thus exhibits favorable lithography properties. In addition, when Rd⁴ is an aromatic group, in lithography using EUV or the like as an exposure light source, the resist composition exhibits excellent light absorption efficiency and exhibits favorable sensitivity and lithography properties.

In Formula (d1-3), Yd¹ is a single bond or a divalent linking group.

The divalent linking group for Yd¹ is not particularly limited, and examples thereof include a divalent hydrocarbon group which may have a substituent (an aliphatic hydrocarbon group, and an aromatic hydrocarbon group), and a heteroatom-containing divalent linking group. Examples of these groups each include the same divalent hydrocarbon group which may have a substituent and heteroatom-containing divalent linking group exemplified in the description of the divalent linking group for Ya²¹ in Formula (a2-1).

Yd¹ is preferably a carbonyl group, an ester bond, an amide bond, an alkylene group or a combination thereof. The alkylene group is more preferably a linear or branched alkylene group and still more preferably a methylene group or an ethylene group.

Preferable specific examples of anion moieties of the component (d1-3) are shown below.

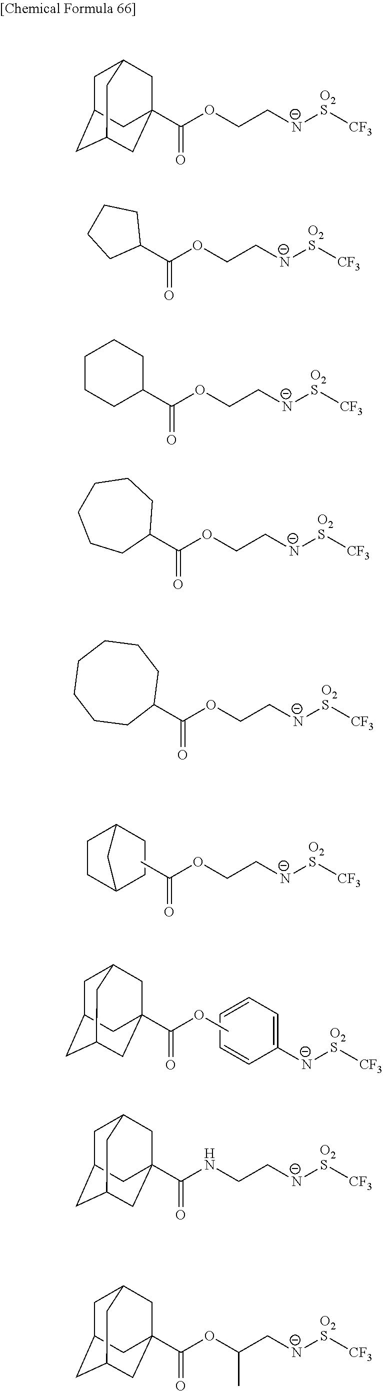

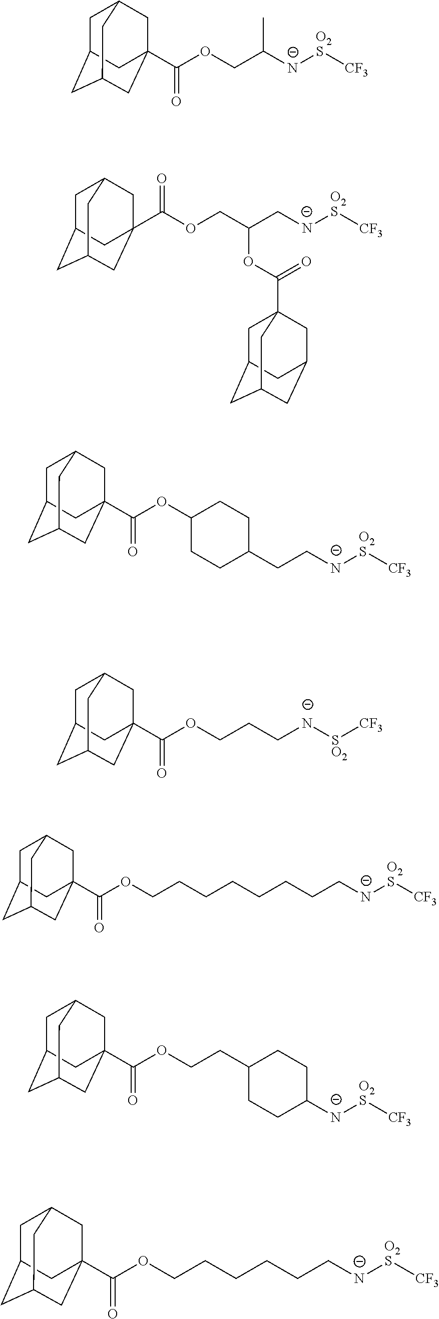

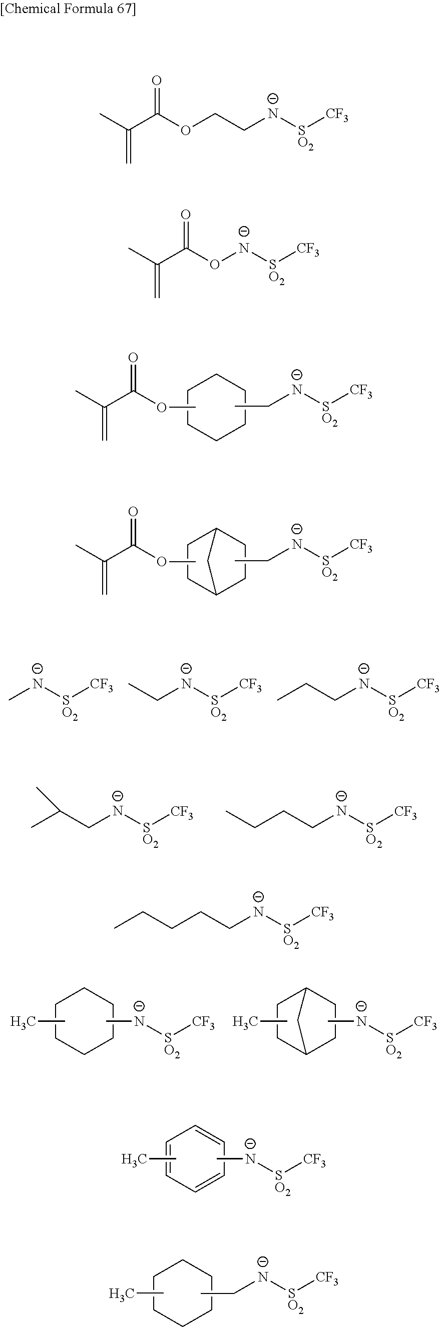

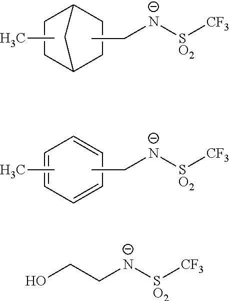

### Cation Moiety

In Formula (d1-3), M^{m+} is an m-valent organic cation, and is the same as M^{m+} in Formula (d1-1).

Components (d1-3) may be used alone or two or more thereof may be used in combination.

As the component (D1), any one of the components (d1-1) to (d1-3) may be used alone or two or more thereof may be used in combination.

When the resist composition contains the component (D1), in the resist composition, the content of the component (D1) with respect to 100 parts by mass of the component (A1) is preferably 0.5 to 15 parts by mass, more preferably 1 to 10 parts by mass, and still more preferably 2 to 8 parts by mass.

When the content of the component (D1) is equal to or larger than the preferable lower limit value, particularly favorable lithography properties and a resist pattern shape are likely to be obtained. On the other hand, when the content is equal to or smaller than the upper limit value, favorable sensitivity can be maintained, and throughput is also excellent.

### Method for Producing Component (D1):

The method for producing the component (d1-1) and the component (d1-2) is not particularly limited, and they can be produced by known methods.

In addition, the method for producing the component (d1-3) is not particularly limited, and the component can be produced by, for example, the same method described in US2012-0149916.

### Component (D2)

The component (D) may contain a nitrogen-containing organic compound component (hereinafter referred to as a “component (D2)”) that does not correspond to the component (D1).

The component (D2) is not particularly limited as long as it functions as an acid diffusion control agent and does not correspond to the component (D1), and any known component may be optionally used. Among these, an aliphatic amine is preferable, and among these, particularly, a secondary aliphatic amine or a tertiary aliphatic amine is more preferable.

The aliphatic amine is an amine having one or more aliphatic groups, and the aliphatic group preferably has 1 to 12 carbon atoms.

Examples of aliphatic amines include amines in which at least one hydrogen atom of ammonia NH₃ is substituted with an alkyl group or hydroxyalkyl group having 12 or fewer carbon atoms (alkylamines or alkyl alcohol amines) and cyclic amines.

Specific examples of alkylamines and alkyl alcohol amines include monoalkylamines such as n-hexylamine, n-heptylamine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylamine, triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, and tri-n-dodecylamine; and alkyl alcohol amines such as diethanolamine, triethanolamine, diisopropanolamine, triisopropanolamine, di-n-octanolamine, and tri-n-octanolamine. Among these, a trialkylamine having 5 to 10 carbon atoms is more preferable, and tri-n-pentylamine or tri-n-octylamine is particularly preferable.

Examples of cyclic amines include a heterocyclic compound containing a nitrogen atom as a heteroatom. The heterocyclic compound may be a monocyclic compound (aliphatic monocyclic amine) or a polycyclic compound (aliphatic polycyclic amine).

Specific examples of aliphatic monocyclic amines include piperidine and piperazine.

The aliphatic polycyclic amine preferably has 6 to 10 carbon atoms, and specific examples thereof include 1,5-diazabicyclo[4.3.0]-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris{2-(2-methoxyethoxy)ethyl}amine, tris{2-(2-methoxyethoxymethoxy)ethyl}amine, tris{2-(1-methoxyethoxy)ethyl}amine, tris{2-(1-ethoxyethoxy)ethyl}amine, tris{2-(1-ethoxypropoxy)ethyl}amine, tris[2-{2-(2-hydroxyethoxy)ethoxy}ethyl]amine, and triethanolamine triacetate, and triethanolamine triacetate is preferable.

In addition, an aromatic amine may be used as the component (D2).

Examples of aromatic amines include 4-dimethylaminopyridine, pyrrole, indole, pyrazole, imidazole, derivatives thereof, tribenzylamine, 2,6-diisopropylaniline, N-tert-butoxycarbonylpyrrolidine, and 2,6-di-tert-butylpyridine.

Among the above examples, the component (D2) is preferably an alkylamine and more preferably a trialkylamine having 5 to 10 carbon atoms.

Components (D2) may be used alone or two or more thereof may be used in combination.

When the resist composition contains the component (D2), in the resist composition, the content of the component (D2) with respect to 100 parts by mass of the component (A1) is preferably 0.01 to 5 parts by mass, more preferably 0.1 to 5 parts by mass, and still more preferably 0.5 to 5 parts by mass.

When the content of the component (D2) is equal to or larger than the preferable lower limit value, particularly favorable lithography properties and a resist pattern shape are likely to be obtained. On the other hand, when the content is equal to or smaller than the upper limit value, favorable sensitivity can be maintained, and throughput is also excellent.

<<At Least One Compound (E) Selected from the Group Consisting of Organic Carboxylic Acid, Phosphorus Oxoacid and Derivatives Thereof>>

In order to prevent deterioration of sensitivity and improve a resist pattern shape and post-exposure temporal stability, the resist composition of the present embodiment can contain, as an optional component, at least one compound (E) selected from the group consisting of an organic carboxylic acid, a phosphorus oxoacid and derivatives thereof (hereinafter referred to as a “component (E)”).

Specific examples of organic carboxylic acids include acetic acid, malonic acid, citric acid, malic acid, succinic acid, benzoic acid, and salicylic acid, and among these, salicylic acid is preferable.

Examples of phosphorus oxoacids include phosphoric acid, phosphonic acid, and phosphinic acid, and among these, phosphonic acid is particularly preferable.

In the resist composition of the present embodiment, the components (E) may be used alone or two or more thereof may be used in combination.

When the resist composition contains the component (E), the content of the component (E) with respect to 100 parts by mass of the component (A1) is preferably 0.01 to 5 parts by mass and more preferably 0.05 to 3 parts by mass. Within the above range, lithography properties are further improved.

### <<Fluorine Additive Component (F)>>

The resist composition of the present embodiment may contain a fluorine additive component (hereinafter referred to as a “component (F)”) as a hydrophobic resin.

The component (F) is used to impart water repellency to the resist film, and when used as a resin separate from the component (A), lithography properties are improved.

As the component (F), for example, fluorine-containing high-molecular-weight compounds described in Japanese Unexamined Patent Application, First Publication No. 2010-002870, Japanese Unexamined Patent Application, First Publication No. 2010-032994, Japanese Unexamined Patent Application, First Publication No. 2010-277043, Japanese Unexamined Patent Application, First Publication No. 2011-13569, and Japanese Unexamined Patent Application, First Publication No. 2011-128226 can be used.

More specifically, the component (F) is, for example, a polymer having a structural unit (f1) represented by the following General Formula (f1-1). The polymer is preferably a polymer (homopolymer) composed of only a structural unit (f1) represented by the following Formula (f1-1); a copolymer of the structural unit (f1) and the structural unit (a1); or a copolymer of the structural unit (f1), a structural unit derived from acrylic acid or methacrylic acid, and the structural unit (a1), and more preferably a copolymer of the structural unit (f1) and the structural unit (a1). Here, the structural unit (a1) that is copolymerized with the structural unit (f1) is preferably a structural unit derived from 1-ethyl-1-cyclooctyl (meth)acrylate or a structural unit derived from 1-methyl-1-adamantyl (meth)acrylate, and more preferably a structural unit derived from 1-ethyl-1-cyclooctyl (meth)acrylate.

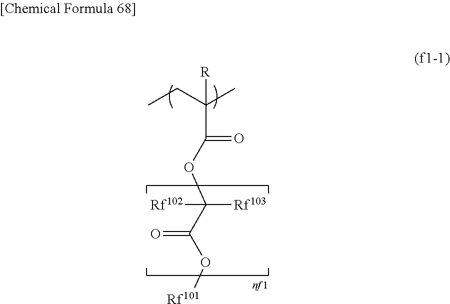

[in the formula, R is the same as defined above, Rf¹⁰² and Rf¹⁰³ are each independently a hydrogen atom, a halogen atom, an alkyl group having 1 to 5 carbon atoms or a halogenated alkyl group having 1 to 5 carbon atoms, Rf¹⁰² and Rf¹⁰³ may be the same as or different from each other, nf¹ is an integer of 0 to 5, and Rf¹⁰¹ is a fluorine atom-containing organic group].

In Formula (f1-1), R bonded to the carbon atom at the α-position is the same as defined above. R is preferably a hydrogen atom or a methyl group.

In Formula (f1-1), the halogen atom for Rf¹⁰² and Rf¹⁰³ is preferably a fluorine atom. Examples of alkyl groups having 1 to 5 carbon atoms for Rf¹⁰² and Rf¹⁰³ include the same alkyl groups having 1 to 5 carbon atoms for R, and a methyl group or an ethyl group is preferable. Specific examples of halogenated alkyl groups having 1 to 5 carbon atoms for Rf¹⁰² and Rf¹⁰³ include groups in which some or all of the hydrogen atoms in an alkyl group having 1 to 5 carbon atoms are substituted with halogen atoms. The halogen atom is preferably a fluorine atom. Among these, Rf¹⁰² and Rf¹⁰³ are preferably a hydrogen atom, a fluorine atom, or an alkyl group having 1 to 5 carbon atoms, more preferably a hydrogen atom, a fluorine atom, a methyl group, or an ethyl group, and still more preferably a hydrogen atom.

In Formula (f1-1), nf¹ is an integer of 0 to 5, preferably an integer of 0 to 3, and more preferably 1 or 2.

In Formula (f1-1), Rf¹⁰¹ is a fluorine atom-containing organic group, and preferably a fluorine atom-containing hydrocarbon group.

The fluorine atom-containing hydrocarbon group may be linear, branched or cyclic, and preferably has 1 to 20 carbon atoms, more preferably has 1 to 15 carbon atoms, and particularly preferably has 1 to 10 carbon atoms.

In addition, in the fluorine atom-containing hydrocarbon group, it is preferable to fluorinate 25% or more of hydrogen atoms in the hydrocarbon group, more preferable to fluorinate 50% or more thereof, and particularly preferable to fluorinate 60% or more thereof because it improves the hydrophobicity of the resist film during immersion exposure.

Among these, Rf¹⁰¹ is more preferably a fluorinated hydrocarbon group having 1 to 6 carbon atoms, and particularly preferably a trifluoromethyl group, —CH₂—CF₃, —CH₂—CF₂—CF₃, —CH(CF₃)₂, —CH₂—CH₂—CF₃, or —CH₂—CH₂—CF₂—CF₂—CF₂—CF₃.

In addition, the component (F) is, for example, a polymer having the structural unit (a8). The polymer is preferably a copolymer of the structural unit (a8) and the structural unit (a2).

The weight average molecular weight (Mw) (in terms of polystyrene determined through gel permeation chromatography) of the component (F) is preferably 1,000 to 50,000, more preferably 5,000 to 40,000, and most preferably 10,000 to 30,000. When the Mw of the component (F) is equal to or smaller than the upper limit value in this range, the component has sufficient solubility in a resist solvent for use as a resist, and when the Mw of the component (F) is equal to or larger than the lower limit value in this range, the resist film has favorable water repellency.

The dispersity (Mw/Mn) of the component (F) is preferably 1.0 to 5.0, more preferably 1.0 to 3.0, and most preferably 1.0 to 2.5.

In the resist composition of the present embodiment, the components (F) may be used alone or two or more thereof may be used in combination.

When the resist composition contains the component (F), the content of the component (F) with respect to 100 parts by mass of the component (A1) is preferably 0.5 to 10 parts by mass and more preferably 1 to 10 parts by mass.

### <<Organic Solvent Component (S)>>

The resist composition of the present embodiment can be produced by dissolving a resist material in an organic solvent component (hereinafter referred to as a “component (S)”).

The component (S) may be any component that can dissolve components to be used and form a uniform solution, and any solvent appropriately selected from among conventionally known solvents for chemically amplified resist compositions can be used.

In the resist composition of the present embodiment, the components (S) may be used alone or a mixed solvent of two or more thereof may be used. Among these, PGMEA, PGME, γ-butyrolactone, ethyl lactate (EL), cyclohexanone, and diacetone alcohol are preferable.

In addition, the component (S) is preferably a mixed solvent in which PGMEA and a polar solvent are mixed. The mixing ratio (mass ratio) may be appropriately determined in consideration of the compatibility between PGMEA and the polar solvent.

In addition, as the component (S), a mixed solvent of at least one selected from among PGMEA and EL and γ-butyrolactone is also preferable. In this case, the mixing ratio (mass ratio) between the former and the latter is preferably 70:30 to 95:5.

The amount of the component (S) used is not particularly limited and is appropriately set according to the coating film thickness at a concentration at which it can be applied to a substrate and the like. Generally, the component (S) is used so that the solid content concentration of the resist composition is within a range of 0.1 to 20 mass %, and preferably 0.2 to 15 mass %.

In the resist composition of the present embodiment, the resist material may be dissolved in the component (S), and impurities and the like may be then removed using the polyimide porous film, the polyamide-imide porous film or the like. For example, the resist composition may be filtered using a filter made of a polyimide porous film, a filter made of a polyamide-imide porous film, a filter made of a polyimide porous film and a polyamide-imide porous film or the like. As the polyimide porous film and the polyamide-imide porous film, for example, those described in Japanese Unexamined Patent Application, First Publication No. 2016-155121 may be exemplified.

The resist composition of the present embodiment described above contains a compound (D0) represented by General Formula (d0) (component (D0)).

In the component (D0), Xd being an iodine atom, a fluorine atom, a bromine atom or a fluorinated alkyl group is bonded as at least one substituent to the aromatic ring Ar of the anion moiety. In Xd, an iodine atom has high absorption of EUV with a wavelength of 13.5 nm. Therefore, the component (D0) is likely to generate secondary electrons during exposure. The secondary electrons generated from the component (D0) upon exposure transfer the energy of the secondary electrons to the component that generates an acid upon exposure (the base component (A) or the acid generator component (B)), and decomposition of the component that generates an acid upon exposure is promoted. As a result, the sensitivity is improved when a resist pattern is formed using the resist composition of the present embodiment.

On the other hand, Xd reduces the solubility in a development solution (particularly an alkaline development solution). However, the component (D0) has a carbonyl group between the aromatic ring Ar and the carboxy group (—C(═O)—O—) in the anion moiety. Therefore, the component (D0) has improved solubility in a development solution (particularly an alkaline development solution).

It is speculated that the above effects combine to allow the resist composition of the present embodiment to achieve high sensitivity, improve the contrast between the exposed part and the unexposed part of the resist film, and improve CDU.

### (Method for Forming Resist Pattern)

A method for forming a resist pattern according to a second aspect of the present invention is a method including a step of forming a resist film on a support using the resist composition according to the first aspect of the present invention, a step of exposing the resist film, and a step of developing the exposed resist film to form a resist pattern.

One embodiment of such a method for forming a resist pattern is, for example, a method for forming a resist pattern performed as follows.

First, the resist composition of the above embodiment is applied onto a support using a spinner or the like and subjected to a bake (post apply bake (PAB)) treatment, for example, under a temperature condition of 80 to 150° C. for 40 to 120 seconds, preferably 60 to 90 seconds, to form a resist film.

Next, the resist film is subjected to exposure using, for example, an exposure device such as an electron beam lithography device or an ArF exposure device, by exposure through a mask (mask pattern) on which a predetermined pattern is formed, or by selective exposure through lithography using direct electron beam emission without using a mask pattern, and then subjected to a bake (post exposure bake (PEB)) treatment, for example, under a temperature condition of 80 to 150° C. for 40 to 120 seconds, preferably for 60 to 90 seconds.

Next, the resist film is subjected to a development treatment. The development treatment is performed using an alkaline development solution in the alkaline development process, and using a development solution containing an organic solvent (organic development solution) in the solvent development process.

After the development treatment, a rinse treatment is preferably performed. In the alkaline development process, the rinse treatment is preferably a water rinse using pure water, and in the solvent development process, a rinse solution containing an organic solvent is preferably used.

In the solvent development process, after the development treatment or the rinse treatment, the development solution or rinse solution adhered to the pattern may be removed using a supercritical fluid.

After the development treatment or after the rinse treatment, drying is performed. In addition, in some cases, a baking treatment (post-baking) may be performed after the development treatment.

The support is not particularly limited, and any conventionally known support can be used, and examples thereof include a substrate for an electronic component and a substrate on which a predetermined wiring pattern is formed. More specific examples thereof include a silicon wafer, a substrate made of a metal such as copper, chromium, iron, or aluminum, and a glass substrate. As the material of the wiring pattern, for example, copper, aluminum, nickel, gold or the like can be used.

The wavelength used for exposure is not particularly limited, and radiation such as ArF excimer laser, KrF excimer laser, F2 excimer laser, extreme ultraviolet (EUV) radiation, vacuum ultraviolet (VUV), electron beam (EB), X-rays, and soft X-rays can be used.

The resist film exposure method may be general exposure (dry exposure) that is performed in air or in an inert gas such as nitrogen, or may be liquid immersion exposure (liquid immersion lithography).

The liquid immersion exposure is an exposure method in which the space between the resist film and the lowest lens of the exposure device is filled in advance with a solvent (liquid immersion medium) that has a larger refractive index than air, and exposure (immersion exposure) is performed in this state.

The liquid immersion medium is preferably a solvent having a larger refractive index than air and having a smaller refractive index than the resist film to be exposed, and examples thereof include water, a fluorine-based inert liquid, a silicone solvent, and a hydrocarbon solvent.

As the liquid immersion medium, water is preferably used.

Examples of alkaline development solutions used in the development treatment in the alkaline development process include a 0.1 to 10 mass % tetramethylammonium hydroxide (TMAH) aqueous solution.

The organic solvent contained in the organic development solution used in the development treatment in the solvent development process may be any solvent that can dissolve the component (A) (the component (A) before exposure), and can be appropriately selected from among known organic solvents. Specific examples thereof include polar solvents such as a ketone solvent, an ester solvent, an alcohol solvent, a nitrile solvent, an amide solvent, and an ether solvent, and a hydrocarbon solvent.

Examples of ester solvents include methyl acetate, butyl acetate, ethyl acetate, isopropyl acetate, pentyl acetate, isopentyl acetate, amyl acetate, propylene glycol monomethyl ether acetate, ethylene glycol monoethyl ether acetate, diethylene glycol monobutyl ether acetate, diethylene glycol monoethyl ether acetate, ethyl 3-ethoxypropionate, 3-methoxybutyl acetate, 3-methyl-3-methoxybutyl acetate, methyl formate, ethyl formate, butyl formate, propyl formate, ethyl lactate, butyl lactate, propyl lactate, butyl butanoate, methyl 2-hydroxyisobutyrate, isoamyl acetate, isobutyl isobutyrate, and butyl propionate.

Examples of nitrile solvents include acetonitrile, propionitrile, valeronitrile, and butyronitrile.

As necessary, known additives can be added to the organic development solution. Examples of additives include a surfactant.

The development treatment can be performed by a known development method, for example, a method of immersing a support in a development solution for a certain time (dip method), a method of raising a development solution on the surface of a support by surface tension and leaving it for a certain time (paddle method), a method of spraying a development solution to the surface of a support (spray method), or a method of continuously applying a development solution by scanning a development solution application nozzle at a certain speed onto a support that rotates at a certain speed (dynamic dispensing method).

As the organic solvent contained in the rinse solution used in the rinse treatment after the development treatment in the solvent development process, for example, one that does not easily dissolve the resist pattern that is appropriately selected from among the organic solvents exemplified as the organic solvents used in the organic development solution can be used. Generally, at least one solvent selected from among a hydrocarbon solvent, a ketone solvent, an ester solvent, an alcohol solvent, an amide solvent and an ether solvent is used.

These organic solvents may be used alone or two or more thereof may be used in combination. In addition, they may be used in combination with other organic solvents or water.

The rinse treatment (washing treatment) using a rinse solution is performed by a known rinse method. Examples of rinse treatment methods include a method of continuously applying a rinse solution onto a support that rotates at a certain speed (rotary application method), a method of immersing a support in a rinse solution for a certain time (dip method), and a method of spraying a rinse solution to the surface of a support (spray method).

It is preferable that the resist composition of the above embodiment and various materials (for example, a resist solvent, a development solution, a rinse solution, a composition for forming an anti-reflective film, and a composition for forming a top coat) used in the method for forming a resist pattern of the above embodiment do not contain impurities such as metals, halogen-containing metal salts, acids, alkalis, and components containing sulfur atoms or phosphorus atoms.

Here, examples of metal atom-containing impurities include Na, K, Ca, Fe, Cu, Mn, Mg, Al, Cr, Ni, Zn, Ag, Sn, Pb, Li, and salts thereof. The content of impurities contained in these materials is preferably 200 ppb or less, more preferably 1 ppb or less, still more preferably 100 ppt (parts per trillion) or less, particularly preferably 10 ppt or less, and most preferably substantially zero (equal to or smaller than the detection limit of the measurement device).

According to the method for forming a resist pattern of the present embodiment described above, since the above resist composition is used, it is possible to achieve high sensitivity and form a resist pattern with favorable CDU.

### (Compound)

The compound of the present embodiment is represented by the following General Formula (d0).

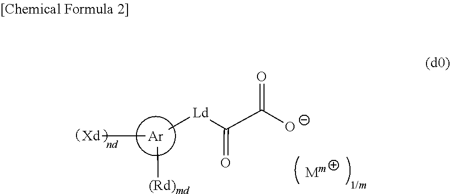

[in the formula, Ar is an aromatic ring; Xd is an iodine atom, a fluorine atom, a bromine atom or a fluorinated alkyl group; Rd is a substituent; nd is an integer of 1 or more as long as the valence allows, and md is an integer of 0 or more as long as the valence allows; Ld is a single bond or a divalent linking group; when nd is an integer of 2 or more, a plurality of Xd's may be the same as or different from each other; when md is an integer of 2 or more, a plurality of Rd's may be the same as or different from each other; and m is an integer of 1 or more, and M^{m+} is an m-valent cation].

The compound of the present embodiment is the same as the component (D0) of the resist composition according to the first aspect.

The compound of the present embodiment is beneficial as the acid diffusion control agent used in the resist composition.

### (Method for Producing Compound)

The method for producing a compound of the present embodiment is not particularly limited, and for example, a compound (X-1) represented by the following General Formula (X-1) and a compound (d0-pre) represented by the following General Formula (d0-pre) are subjected to a salt exchange reaction in the presence of a base to obtain a compound represented by General Formula (d0) (hereinafter referred to as a “compound (d0)”).

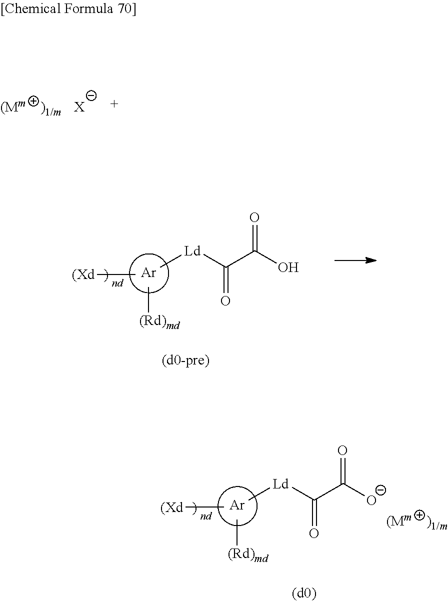

[in the formula, Ar is an aromatic ring; Xd is an iodine atom, a fluorine atom, a bromine atom or a fluorinated alkyl group; Rd is a substituent; nd is an integer of 1 or more as long as the valence allows, and md is an integer of 0 or more as long as the valence allows; Ld is a single bond or a divalent linking group; when nd is an integer of 2 or more, a plurality of Xd's may be the same as or different from each other; when md is an integer of 2 or more, a plurality of Rd's may be the same as or different from each other; and m is an integer of 1 or more, M^{m+} is an m-valent cation, and X⁻ is a halogen anion].

In Formula (d0-pre), Ar, Xd, Rd, nd, md and Ld are the same as Ar, Xd, Rd, nd, md and Ld in Formula (d0).

In Formula (X-1), m and M′^{m+} are the same as m and M′^{m+} in Formula (d0).

In Formula (X-1), X⁻ is preferably a bromide ion or a chloride ion, and more preferably a bromide ion.

Examples of bases used in the salt exchange reaction between the compound (X-1) and a compound represented by the following General Formula (d0-pre) include tetramethylammonium hydroxide (TMAH), sodium hydride, K₂CO₃, Cs₂CO₃, lithium diisopropylamide (LDA), triethylamine, and 4-dimethylaminopyridine.

Examples of reaction solvents include water, dichloromethane, acetonitrile, and chloroform.

In the salt exchange reaction, the reaction temperature is, for example, 0 to 100° C., and the reaction time is, for example, 10 minutes or longer and 24 hours or shorter.

After the salt exchange reaction is completed, the compound (d0) in the reaction solution may be isolated and purified. For isolation and purification, conventionally known methods can be used, and for example, an appropriate combination of concentration, solvent extraction, distillation, crystallization, recrystallization, and chromatography can be used.

The structure of the compound (d0) obtained as described above can be identified by a general organic analysis method such as ¹H-nuclear magnetic resonance (NMR) spectroscopy, ¹³C-NMR spectroscopy, ¹⁹F-NMR spectroscopy, infrared absorption (IR) spectroscopy, mass spectrometry (MS), an elemental analysis method, or an X-ray crystal diffraction method.

### EXAMPLES

The present invention will be described in more detail below with reference to examples, but the present invention is not limited to these examples.

### Production Examples of Compounds

### Production Example 1: Production of Compound (D0-1)

4-Iodophenylglyoxylic acid (2.3 g, 8.4 mmol) and the compound A1 (2.9 g, 8.4 mmol) were dissolved in dichloromethane (35 g), a 5% tetramethylammonium hydroxide (TMAH) aqueous solution (15.3 g) was added, and the mixture was reacted at room temperature for 30 minutes. After the reaction was completed, an aqueous phase was removed, and an organic phase was washed 10 times with ultrapure water (10 g). The organic phase was concentrated and dried using a rotary evaporator to obtain a compound (D0-1) (3.1 g, yield=85.0%).

The ¹H NMR measurement results of the compound (D0-1) are shown below. ¹HNMR(400 MHz, DMSO-d6) δ (ppm)=7.74-7.90(m,ArH,ArH—I,17H), 6.89(dd,ArH—I,2H)

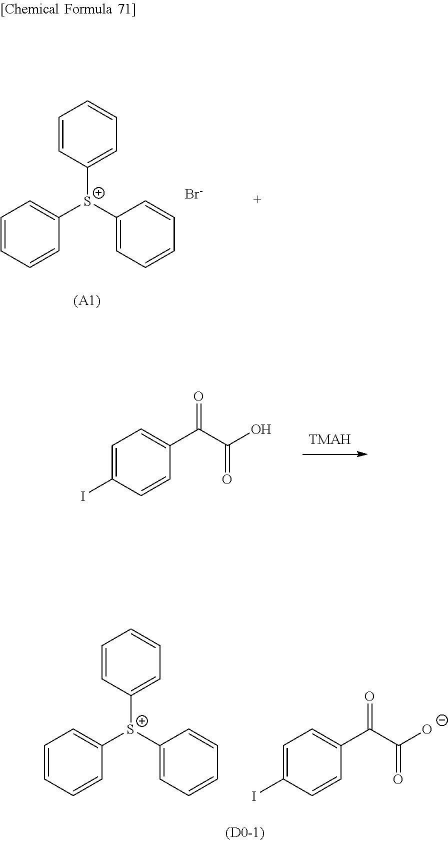

### Production Examples 2 to 6: Production of Compounds (D0-2) to (D0-06)

Compounds (D0-2) to (D0-06) were obtained in the same manner as in the above “Production Example 1: production of compound (D0-1)” except that combinations of the following compounds A2 to A3 and the following phenylglyoxylic acids having various iodines and hydroxy groups (compounds B1 to B3) were changed.

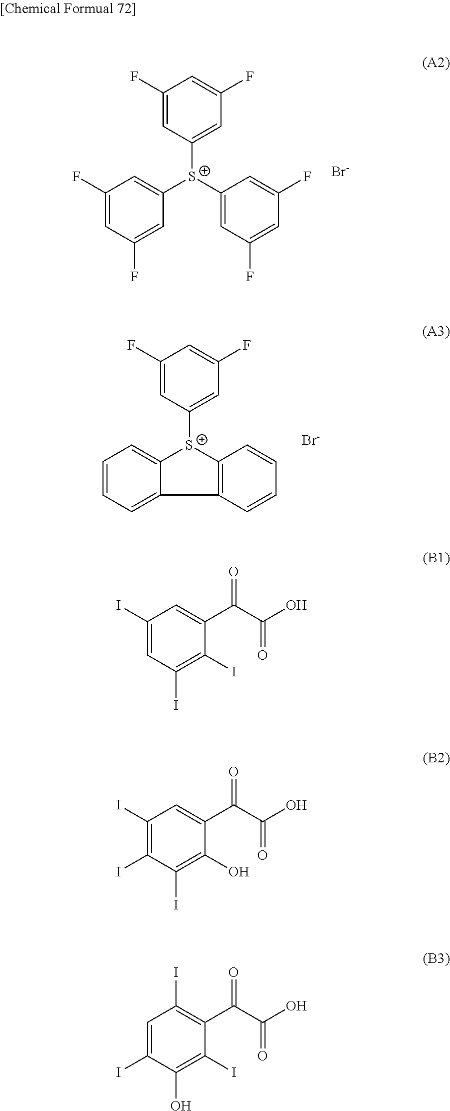

The ¹H NMR measurement results of the compound (D0-2) are shown below. ¹H NMR (400 MHz, DMSO-d6) δ (ppm)=8.44(d,ArH—I,1H), 7.74-7.90(m,ArH,ArH—I,16H)

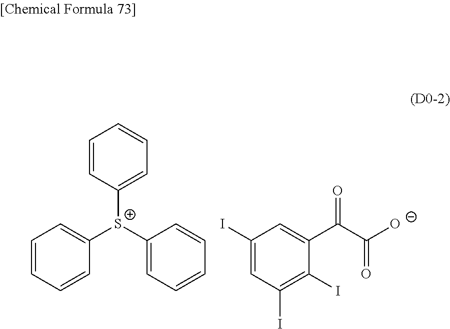 [SMILES: c1ccc([S+](c2ccccc2)c2ccccc2)cc1 ; O=C([O-])C(=O)c1cc(I)cc(I)c1I]

The ¹H NMR measurement results of the compound (D0-3) are shown below. ¹H NMR (400 MHz, DMSO-d6) δ (ppm)=10.91(s,OH,1H), 8.44(d,ArH—I,1H), 7.74-7.90(m,ArH,15H), 7.40(d,ArH—I,1H)

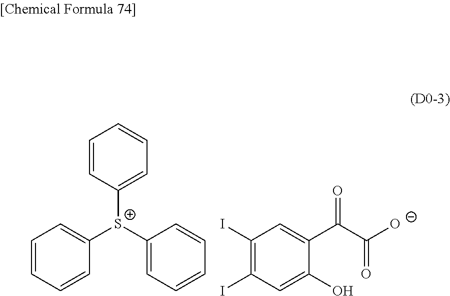 [SMILES: c1ccc([S+](c2ccccc2)c2ccccc2)cc1 ; O=C([O-])C(=O)c1cc(I)c(I)cc1O]

The ¹H NMR measurement results of the compound (D0-4) are shown below. ¹H NMR (400 MHz, DMSO-d6) δ (ppm)=9.92(s,OH,1H), 8.12(d,ArH—I,1H), 7.74-7.90(m,ArH,15H)

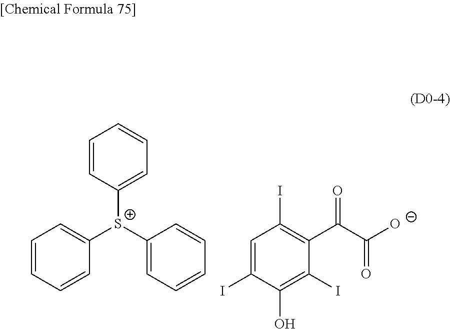 [SMILES: c1ccc([S+](c2ccccc2)c2ccccc2)cc1 ; O=C([O-])C(=O)c1c(I)cc(I)c(O)c1I]

The ¹H NMR measurement results of the compound (D0-5) are shown below. ¹H NMR (400 MHz, DMSO-d6) δ (ppm)=9.92(s,OH,1H), 8.12(d,ArH—I,1H), 7.77-7.98(m,ArH,9H)

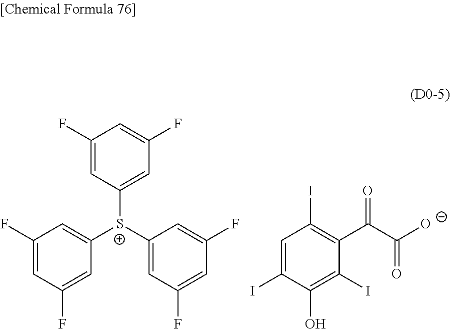 [SMILES: Fc1cc(F)cc([S+](c2cc(F)cc(F)c2)c2cc(F)cc(F)c2)c1 ; O=C([O-])C(=O)c1c(I)cc(I)c(O)c1I]

The ¹H NMR measurement results of the compound (D0-6) are shown below. ¹HNMR(400 MHz, DMSO-d6) δ (ppm)=9.92(s,OH,1H), 8.51(d,ArH,2H), 8.41(d,ArH,2H), 8.12(d,ArH—I,1H), 7.98(t,ArH,2H), 7.76(t,ArH,2H), 7.69(tt,ArH,1H), 7.49(d,ArH,2H)

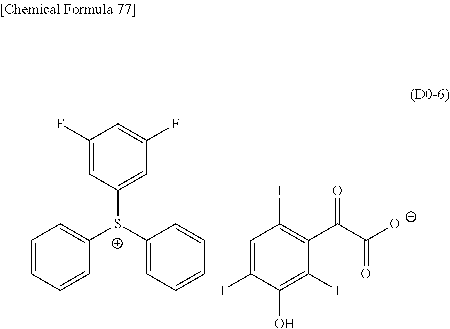 [SMILES: Fc1cc(F)cc([S+](c2ccccc2)c2ccccc2)c1 ; O=C([O-])C(=O)c1c(I)cc(I)c(O)c1I]

### Preparation of Resist Composition

### Examples 1 to 6 and Comparative Examples 1 to 5

The components shown in Table 1 were mixed and dissolved to prepare resist compositions of examples.

TABLE 1

| | Component | Component | Component | Component | Component |
|---|---|---|---|---|---|
| | (A) | (B) | (D) | (F) | (S) |
| Example 1 | (A)-1 | (B)-1 | (D0)-1 | (F)-1 | (S)-1 |
| | [100] | [15] | [5] | [5] | [6000] |
| Example 2 | (A)-1 | (B)-1 | (D0)-2 | (F)-1 | (S)-1 |
| | [100] | [15] | [5] | [5] | [6000] |
| Example 3 | (A)-1 | (B)-1 | (D0)-3 | (F)-1 | (S)-1 |
| | [100] | [15] | [5] | [5] | [6000] |
| Example 4 | (A)-1 | (B)-1 | (D0)-4 | (F)-1 | (S)-1 |
| | [100] | [15] | [5] | [5] | [6000] |
| Example 5 | (A)-1 | (B)-1 | (D0)-5 | (F)-1 | (S)-1 |
| | [100] | [15] | [5] | [5] | [6000] |
| Example 6 | (A)-1 | (B)-1 | (D0)-6 | (F)-1 | (S)-1 |
| | [100] | [15] | [5] | [5] | [6000] |
| Example 7 | (A)-1 | (B)-1 | (D0)-6 | — | (S)-1 |
| | [100] | [15] | [5] | | [6000] |
| Comparative | (A)-1 | (B)-1 | (D1)-1 | (F)-1 | (S)-1 |
| Example 1 | [100] | [15] | [5] | [5] | [6000] |
| Comparative | (A)-1 | (B)-1 | (D1)-2 | (F)-1 | (S)-1 |
| Example 2 | [100] | [15] | [5] | [5] | [6000] |
| Comparative | (A)-1 | (B)-1 | (D1)-3 | (F)-1 | (S)-1 |
| Example 3 | [100] | [15] | [5] | [5] | [6000] |
| Comparative | (A)-1 | (B)-1 | (D1)-4 | (F)-1 | (S)-1 |
| Example 4 | [100] | [15] | [5] | [5] | [6000] |
| Comparative | (A)-1 | (B)-1 | (D1)-5 | (F)-1 | (S)-1 |
| Example 5 | [100] | [15] | [5] | [5] | [6000] |

The abbreviations in Table 1 have the following meanings. The numbers in [ ] are the amounts used (parts by mass).
(A)-1: high-molecular-weight compound represented by the following Chemical Formula
(A1)-1. Regarding the high-molecular-weight compound (A1)-1, the weight average molecular weight (Mw) in terms of standard polystyrene determined through GPC measurement was 5,100, and the molecular weight dispersity (Mw/Mn) was 1.65. The copolymer composition ratio (the ratio (molar ratio) between the structural units in the structural formula) determined through ¹³C-NMR was l/m=40/60.

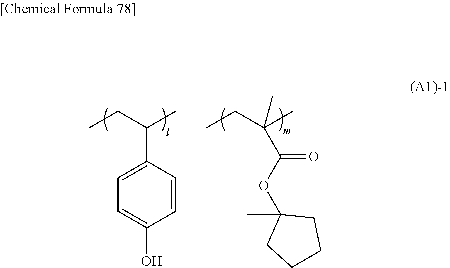

(B)-1: acid generator containing the following compound (B1-1).

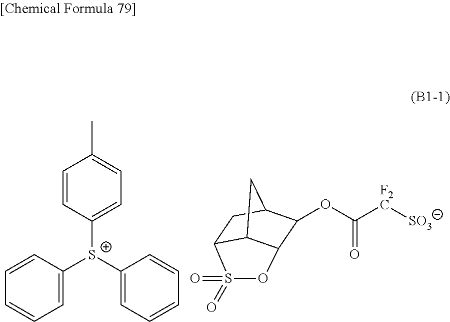

(D0)-1: acid diffusion control agent containing the compound (D0-1).
(D0)-2: acid diffusion control agent containing the compound (D0-2).
(D0)-3: acid diffusion control agent containing the compound (D0-3).
(D0)-4: acid diffusion control agent containing the compound (D0-4).
(D0)-5: acid diffusion control agent containing the compound (D0-5).
(D0)-6: acid diffusion control agent containing the compound (D0-6).
(D1)-1: acid diffusion control agent containing the following compound (D1-1).
(D1)-2: acid diffusion control agent containing the following compound (D1-2).
(D1)-3: acid diffusion control agent containing the following compound (D1-3).
(D1)-4: acid diffusion control agent containing the following compound (D1-4).
(D1)-5: acid diffusion control agent containing the following compound (D1-5).

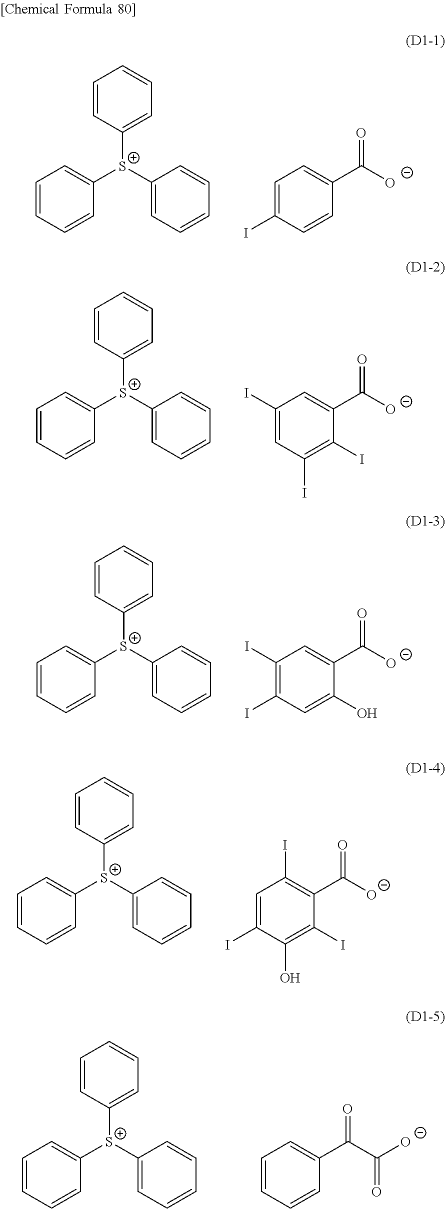

(F)-1: fluorine-containing high-molecular-weight compound represented by the following Chemical Formula (F-1). The mass average molecular weight (Mw) in terms of standard polystyrene determined through GPC measurement was 15,000, and the molecular weight dispersity (Mw/Mn) was 1.70. The copolymer composition ratio (the ratio (molar ratio) between the structural units in the structural formula) determined through ¹³C-NMR was l/m=70/30.

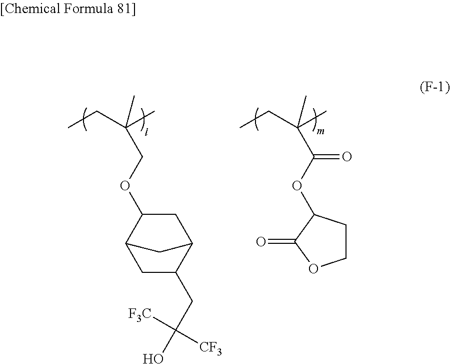

(S)-1: a mixed solvent of propylene glycol monomethyl ether acetate/propylene glycol monomethyl ether/ethyl lactate/diacetone alcohol=45/25/15/15 (mass ratio).

### <Formation of Resist Pattern>

A resist composition of each example was applied onto a 12-inch silicon substrate on which a hexamethyldisilane (HMDS) treatment was performed using a spinner, a pre-bake (PAB) treatment was performed on a hot plate at a temperature of 110° C. for 60 seconds, and drying was performed to form a resist film with a film thickness of 50 nm.

Next, the resist film was exposed to light using an EUV scanner NXE3400 (NXE3400 (NA0.33, inner σ/outer σ=0.3/0.9, quadruple illumination), commercially available from ASML) to form a contact hole pattern (hereinafter referred to as a “CH pattern”) in which holes with a diameter of 26 nm were arranged at equal intervals (a pitch of 52 nm), and then subjected to a post exposure bake (PEB) treatment at 100° C. for 60 seconds.

Next, alkaline development was performed using a 2.38 mass % tetramethylammonium hydroxide (TMAH) aqueous solution “NMD-3” (product name, commercially available from Tokyo Ohka Kogyo Co., Ltd.) at 23° C. for 30 seconds, and a water rinse treatment was then performed using pure water for 15 seconds. As a result, a CH pattern in which holes with a diameter of 26 nm were arranged at equal intervals (a pitch of 52 nm) was formed.

### [Evaluation of Optimal Exposure Amount (Eop)]

The optimal exposure amount Eop (mJ/cm²) for forming a CH pattern with a target size by the above method for forming a resist pattern was determined. The results are shown in Table 2 as “Eop (mJ/cm²)”.

[Evaluation of in-Plane Uniformity (CDU) of Pattern Dimension]

Regarding the CH pattern formed by the above <Formation of resist pattern>, the CH pattern was observed from above using a length measuring SEM (scanning electron microscope, an acceleration voltage of 500 V, product name: CG6300, commercially available from Hitachi High-Tech Corporation), and the hole diameter (nm) of each hole was measured. Then, three times (3σ) the standard deviation (σ) calculated from the measurement result was determined. The results are shown in Table 2 as “CDU(nm).”

A smaller value of 3σ obtained in this manner indicates higher dimension (CD) uniformity of the plurality of holes formed in the resist film.

TABLE 2

| | Eop (mJ/cm²) | CDU (nm) |
|---|---|---|
| Example 1 | 34 | 2.2 |
| Example 2 | 28 | 2.8 |
| Example 3 | 29 | 2.4 |
| Example 4 | 26 | 2.7 |
| Example 5 | 23 | 2.8 |
| Example 6 | 25 | 2.9 |
| Example 7 | 25 | 3.0 |
| Comparative Example 1 | 36 | 3.0 |
| Comparative Example 2 | 30 | 3.5 |
| Comparative Example 3 | 32 | 3.1 |
| Comparative Example 4 | 29 | 3.2 |
| Comparative Example 5 | 48 | 4.8 |

As shown in Table 2, the resist compositions of the examples were superior in both the sensitivity and CDU to the resist compositions of the comparative examples.

While preferable examples of the present invention have been described above, the present invention is not limited to these examples. Additions, omissions, substitutions and other modifications of the configuration can be made without departing from the scope of the present invention. The present invention is not limited to the above descriptions, but it is only limited by the scope of the appended claims.

What is claimed is:

1. A resist composition which generates an acid upon exposure and its solubility in a development solution changes under the action of the acid, comprising:
a resin component (A1) whose solubility in a development solution changes under the action of the acid; and
a compound (D0) represented by the following General Formula (d0): 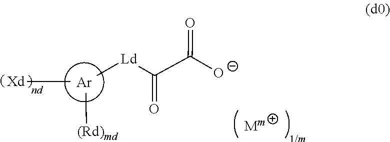
wherein Ar is an aromatic ring; Xd is an iodine atom, a fluorine atom, a bromine atom or a fluorinated alkyl group; Rd is a substituent; nd is an integer of 1 or more as long as the valence allows, and md is an integer of 0 or more as long as the valence allows; Ld is a single bond or a divalent linking group; when nd is an integer of 2 or more, a plurality of Xd's may be the same as or different from each other; when md is an integer of 2 or more, a plurality of Rd's may be the same as or different from each other; and m is an integer of 1 or more, and M^{m+} is an m-valent cation.

2. The resist composition according to claim 1, wherein, in General Formula (d0), Ld is a single bond.

3. The resist composition according to claim 1, wherein, in General Formula (d0), Rd is a hydroxy group, and md is an integer of 1 or more as long as the valence allows.

4. The resist composition according to claim 1, wherein, in General Formula (d0), Xd is an iodine atom.

5. A method for forming a resist pattern, comprising:
forming a resist film on a support using the resist composition according to claim 1;
exposing the resist film; and
developing the exposed resist film to form a resist pattern.

6. The method for forming a resist pattern according to claim 5, wherein the resist film is exposed to extreme ultraviolet radiation or an electron beam.

7. A compound represented by the following General Formula (d0); 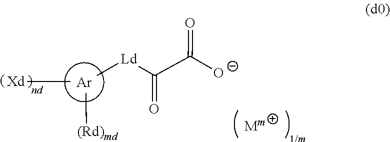
wherein Ar is an aromatic ring; Xd is an iodine atom, a fluorine atom, a bromine atom or a fluorinated alkyl group; Rd is a substituent; nd is an integer of 1 or more as long as the valence allows, and md is an integer of 0 or more as long as the valence allows; Ld is a single bond or a divalent linking group; when nd is an integer of 2 or more, a plurality of Xd's may be the same as or different from each other; when md is an integer of 2 or more, a plurality of Rd's may be the same as or different from each other; and m is an integer of 1 or more, and M^{m+} is an m-valent cation.

8. The compound according to claim 7, wherein, in General Formula (d0), Ld is a single bond.

9. The compound according to claim 7, wherein, in General Formula (d0), Rd is a hydroxy group, and md is an integer of 1 or more as long as the valence allows.

10. The compound according to claim 7, wherein, in General Formula (d0), Xd is an iodine atom.

11. An acid diffusion control agent comprising the compound according to am claim 7.
